(19) United States
(12) Patent Application Publication
(10) Pub. No.: US 2026/0294901 A1
(43) Pub. Date: Oct. 1, 2026

(54) METHODS FOR TREATING MAJOR DEPRESSIVE DISORDER COMPRISING ADMINISTERING LUMATEPERONE

(71) Applicant: INTRA-CELLULAR THERAPIES, INC., Bedminster, NJ (US)

(72) Inventors: Robert E. DAVIS, San Diego, CA (US); Suresh DURGAM, Bedminster, NJ (US); Willie EARLEY, West Chester, PA (US); Susan KOZAUER, Poolesville, MD (US); Hassan LAKKIS, Clark, NJ (US); Changzheng CHEN, Titusville, NJ (US)

(21) Appl. No.: 19/636,856

(22) Filed: Apr. 1, 2026

Related U.S. Application Data
(60) Provisional application No. 63/781,915, filed on Apr. 1, 2025.
(60) Provisional application No. 63/807,486, filed on May 16, 2025.
(60) Provisional application No. 63/823,638, filed on Jun. 13, 2025.
(60) Provisional application No. 63/892,902, filed on Oct. 3, 2025.
(60) Provisional application No. 63/895,594, filed on Oct. 8, 2025.
(60) Provisional application No. 63/912,291, filed on Nov. 5, 2025.
(60) Provisional application No. 63/957,222, filed on Jan. 9, 2026.

(51) Int. Cl. A61K 31/4985; A61K 31/137; A61K 31/55; A61K 45/06; A61P 25/22; A61P 25/24

(52) U.S. Cl. CPC A61K 31/4985; A61K 31/137; A61K 31/55; A61K 45/06; A61P 25/22; A61P 25/24

(57) ABSTRACT

The present disclosure provides methods for the treatment of major depressive disorder (MDD), comprising the administration of pharmaceutical capsules comprising 42 mg of lumateperone (60 mg of lumateperone tosylate) once daily to a patient in need thereof, as well as reduced dosages therefore for certain patients, wherein the treatment is optionally adjunctive to therapy with antidepressants (e.g., SSRIs).

### CROSS-REFERENCE TO RELATED APPLICATIONS

This application claims priority to, and the benefit of, U.S. Provisional Applications Ser. No. 63/781,915, filed on Apr. 1, 2025, 63/807,486, filed on May 16, 2025, 63/823,638, filed on Jun. 13, 2025, 63/892,902, filed on Oct. 3, 2025, 63/895,594, filed on Oct. 8, 2025, 63/912,291, filed on Nov. 5, 2025, and 63/957,222, filed on Jan. 9, 2026, the contents of each of which are hereby incorporated by reference in their entireties.

### TECHNICAL FIELD

The present disclosure relates to the use of lumateperone in the treatment of major depressive disorder (MDD), optionally in combination with antidepressant treatment.

### BACKGROUND

Substituted heterocycle fused gamma-carbolines such as lumateperone are known to be 5-HT_{2A} or 5-HT_{2A}/D2 receptor ligands, which are useful in treating central nervous system disorders. These compounds antagonize the serotonin-2A (5-HT_{2A}) receptor, and/or modulate dopamine receptor signaling at the level of key intra-cellular phosphoproteins. Such compounds are principally known to be useful for the treatment of positive and negative symptoms of schizophrenia. At dopamine D2 receptors, these compounds have dual properties and act as both post-synaptic antagonists and pre-synaptic partial agonists. They also stimulate phosphorylation of glutamatergic NMDA NR2B, or GluN2B, receptors in a mesolimbic specific manner. It is believed that this regional selectivity in the brain areas thought to mediate the efficacy of antipsychotic drugs, together with the serotonergic, glutamatergic, and dopaminergic interactions, may result in antipsychotic efficacy for positive, negative, affective and cognitive symptoms associated with schizophrenia. The compounds also exhibit serotonin reuptake inhibition, providing antidepressant activity for the treatment of schizoaffective disorder, co-morbid depression, and/or as a stand-alone treatment for major depressive disorder. The 5-HT_{2A} or 5-HT_{2A}/D2 receptor ligands as described are also useful for the treatment of bipolar disorder and other psychiatric and neurodegenerative disorders, particularly behavioral disturbances associated with dementia, autism and other CNS diseases. These features may be able to improve the quality of life of patients with schizophrenia and enhance social function to allow them to more fully integrate into their families and their workplace. These compounds display differential dose-dependent effects, selectively targeting the 5-HT_{2A} receptor at low doses, while progressively interacting with the D2 receptor at higher doses. As a result, at lower doses, they are useful in treating sleep, aggression and agitation. At a high-dose, they can treat acute exacerbated and residual schizophrenia, bipolar disorders, and mood disorders.

Lumateperone, having the formula:

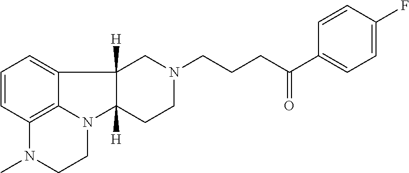 [SMILES: CN1CCN2c3c(cccc31)[C@@H]1CN(CCCC(=O)c3ccc(F)cc3)CC[C@@H]12]

is a novel therapeutic agent with potent (Ki=0.5 nM) 5-HT_{2A} receptor antagonism, activity as a mesolimbic/mesocortical-selective dopamine receptor protein phosphorylation modulator consistent with presynaptic D2 receptor partial agonism and postsynaptic D2 receptor antagonism (Ki=32 nM) in vivo, high D1 receptor affinity (Ki=52 nM), and inhibition of the serotonin transporter (SERT) (Ki=26-62 nM, using different assays for SERT activity). Lumateperone has been approved in the United States for the treatment for schizophrenia and bipolar depression in adults, and it is in clinical development for the treatment of other neuropsychiatric disorders, such as major depressive disorder (MDD), agitation in dementia, including Alzheimer's Disease.

Lumateperone and related compounds have been disclosed in U.S. Pat. Nos. 6,548,493; 7,238,690; 6,552,017; 6,713,471; U.S. RE39,680, and U.S. RE39,679, as novel compounds useful for the treatment of disorders associated with 5-HT_{2A} receptor modulation such as anxiety, depression, psychosis, schizophrenia, sleep disorders, sexual disorders, migraine, conditions associated with cephalic pain, and social phobias. WO 2000/077002 and U.S. Pat. No. 7,071,186, also disclose methods of making substituted heterocycle fused gamma-carbolines and uses of these gamma-carbolines as serotonin agonists and antagonists useful for the control and prevention of central nervous system disorders such as addictive behavior and sleep disorders. WO 2009/145900 and U.S. Pat. No. 8,598,119, and WO 2013/155504 and U.S. Pat. No. 11,053,245, and WO 2013/155506 and U.S. Pat. No. 11,124,514, each incorporated herein by reference, disclose the use of specific substituted heterocycle fused gamma-carbolines for the treatment of a combination of psychosis and depressive disorders as well as sleep, depressive and/or mood disorders in patients with psychosis or Parkinson's disease, for the treatment of post-traumatic stress disorder, impulse control disorders and related disorders, and for the treatment or prophylaxis of disorders associated with dementia, particularly behavioral or mood disturbances such as agitation, irritation, aggressive/assaultive behavior, anger, physical or emotional outbursts and psychosis and sleep disorders associated with dementia. WO 2009/114181 and U.S. Pat. No. 8,648,077, each incorporated herein by reference, disclose methods of preparing toluenesulfonic acid addition salt crystals of particular substituted heterocycle fused gamma-carbolines, e.g., toluenesulfonic acid addition salt of 4-((6bR,10aS)-3-methyl-2,3,6b,9,10,10a-hexahydro-1H-pyrido[3′,4′: 4,5]pyrrolo[1,2,3-de]quinoxaline-8(7H)-yl)-1-(4-fluorophenyl)-1-butanone.

WO 2011/133224 and U.S. Pat. No. 8,993,572, each incorporated herein by reference, disclose prodrugs/metabolites of substituted heterocycle fused gamma-carboline for improved formulation, e.g., extended/controlled release formulation. This application discloses that heterocycle fused gamma-carbolines which are N-substituted with a 4-fluorophenyl(4-hydroxy)butyl moiety are shown to have high selectivity for the serotonin transporter (SERT) relative to the heterocycle fused gamma-carboline containing 4-fluorophenylbutanone.

WO 2009/145900 and U.S. Pat. No. 8,598,119 also disclose that selected substituted heterocycle fused gamma-carboline compounds have nanomolar affinity for the serotonin reuptake transporter (SERT) and so are selective serotonin reuptake inhibitors.

WO2019/178484 and US 2021/0060009, the contents of each of which are incorporated by reference in their entireties, disclose the use of lumateperone and related analogs for the treatment of acute depression and acute anxiety. Unlike traditional antidepressants, including selective serotonin reuptake inhibitors (SSRIs), such as sertraline, escitalopram, fluoxetine, paroxetine, and citalopram, which generally take weeks or months to achieve their full effects (SSRIs typically have an onset of action 3-4 weeks after initiation of daily dosing), lumateperone is believed to achieve rapid efficacy, in as little as a week or less, and even an immediate onset of action (e.g., hours to days after initial dosing). In this regard, lumateperone shares the functional benefits of ketamine. Ketamine has recently been tested as a rapid-acting antidepressant for treatment-resistant depression, in bipolar disorder and major depressive disorder, but it has significant side effects and risk of overdose, and it is not orally active. Lumateperone has shown promise as an orally-available, rapid-acting treatment for depression and anxiety, alone or in conjunction with other anti-anxiety or anti-depressant drugs, such as in treating acute depression and acute anxiety with the rapid onset characteristic of ketamine, but without ketamine's side effects or lack of oral activity. It is believed that these effects are mediated through indirect dopamine D1 receptor-dependent enhancement of NMDA and AMPA currents coupled with activation of the mTOR (e.g., mTORC1) signaling pathway and paralleled by anti-inflammatory properties.

In addition, unlike benzodiazepine class agents, lumateperone appears to be non-addictive, and therefore, particularly suitable for the treatment of acute depressive episodes, including suicidal ideation and severe acute depression and/or severe acute anxiety.

WO 2021/007245 discloses that lumateperone is particularly useful to treat Bipolar II Disorder. Clinical results suggest that lumateperone is at least as effective in this indication as existing antipsychotic agents in treating bipolar disorders, with particularly unexpected efficacy in Bipolar II Disorder, which is often resistant to treatment with antipsychotic agents. Moreover, lumateperone exhibits a favorable safety profile. Clinical trials demonstrate that, unlike many other antipsychotic agents, lumateperone does not increase akathisia, restlessness, or other movement disorders, it does not increase suicidal ideation, and it does not have significant metabolic side effects.

PCT/US2023/067204 discloses that substituted heterocycle fused gamma-carbolines, particularly lumateperone, are effective in reducing aberrantly elevated levels of proinflammatory cytokines in both brain and serum, alters key pathways involved in tissue integrity and the maintenance of the blood-brain barrier (BBB), conferred anxiolytic and antianhedonic properties in rats, and reinforced BBB protection during inflammatory and stress challenges. This suggests that such compounds will be effective in the treatment of psychiatric disorders caused by viral, bacterial, or autoimmune encephalitis, and for treatment of psychiatric symptoms of viral, bacterial, and autoimmune encephalitis. It is further disclosed that such compounds will be effective in protecting or reinforcing the blood-brain barrier.

Lumateperone was found to have the potential to ameliorate pathological levels of inflammation in brain, microglia, and serum, and to preserve the integrity of the BBB following immunological insult and stress in rodents. When administered over a range of doses at different time points, lumateperone reduced key pro-inflammatory markers elevated by an inflammogen (e.g., lipopolysaccharide, LPS) or by acute restraint stress. Surprisingly, the cytokines IL-1β, IL-6, and TNF-α that are normalized by lumateperone treatment are known to be elevated in patients with psychiatric disorders, and in human post-mortem tissues including prefrontal cortex from suicide victims. Lumateperone treatment also reduced expression of the Nlrp3 inflammasome, which is a large multiprotein complex containing NLRP3, a cytosolic sensor involved in innate immunity. Although the inflammasome has no baseline activity, once activated by stress, infections, or other stimuli, the complex is believed to generate active forms of the inflammatory cytokines IL-1β and IL-18. In preclinical studies, Nlrp3-null mutant mice were reported to be resilient to the effects of stress on depression-like behavior, and Nlrp3 expression was increased in peripheral blood mononuclear cells (PBMCs) from untreated patients with MDD. It is postulated that since lumateperone treatment decreases Nlrp3 transcript levels under conditions evoking pathological inflammation, this may contribute, in part, to the antidepressant-like action of lumateperone. In addition, it was shown that lumateperone has anxiolytic-like effects and reverses anhedonia in rats.

Lumateperone has already been approved by the U.S. FDA for the treatment of schizophrenia and depressive episodes associated with bipolar disorder, under the brand name Caplyta®, and it undergoing clinical study for the treatment of major depressive disorder and other disorders.

According to the National Institute of Mental Health, bipolar disorder, also known as manic-depressive illness, is a brain disorder that causes unusual shifts in mood, energy, activity levels, and the ability to carry out day-to-day tasks. There are four basic types of bipolar disorder; all of them involve clear changes in mood, energy, and activity levels. These moods range from periods of extremely “up,” elated, and energized behavior (known as manic episodes) to very sad, “down,” or hopeless periods (known as depressive episodes). Less severe manic periods are known as hypomanic episodes. The two most common bipolar disorders are Bipolar I disorder and Bipolar II disorder, and Caplyta® has been approved the treatment of depressive episodes associated with both, although its efficacy in treating bipolar II disorder is particularly unexpected. Bipolar I disorder is defined by manic episodes that last at least 7 days, or by manic symptoms that are so severe that the person needs immediate hospital care. Usually, depressive episodes occur as well, typically lasting at least 2 weeks. Episodes of depression with mixed features (having depression and manic symptoms at the same time) are also possible. In contrast, Bipolar II disorder is defined by a pattern of depressive episodes and hypomanic episodes, but not the full-blown manic episodes of Bipolar I disorder.

Major depressive disorder (MDD) is another serious depressive illness, but it differs from bipolar disorder in the general absence of manic episodes, and in having other specific features not included in bipolar disorder. According to the Diagnostic and Statistical Manual of Psychiatric Disorder, 5^{th} Edition (Text Revision, 2022) (DSM-5-TR), a patient can be diagnosed as experiencing a major depressive episode if:
(A) The patient has five or more of the following symptoms during the same two-week period and this represent a change from previous functioning, provided that at least one of the symptoms is either (1) depressed mood or (2) loss of interest or pleasure (not counting any symptoms that are clearly attributable to another medical condition):
   (1) depressed mood (or irritable mood, for children and adolescents) most of the day, nearly every day, as indicated by either subjective report (e.g., feels sad, empty, hopeless) or observations made by others (e.g., appears tearful);
   (2) markedly diminished interest or pleasure in all, or almost all, activities most of the day, nearly every day (as indicated by either subjective account or observation);
   (3) significant weight loss when not dieting or weight gain (e.g., a change of more than 5% of body weight in a month), or decrease or increase in appetite nearly every day in children, this can be satisfied by failure to make expected weight gain);
   (4) insomnia or hypersomnia nearly every day;
   (5) psychomotor agitation or retardation nearly every day (observable by others, not merely subjective feelings of restlessness or being slowed down);
   (6) fatigue or loss of energy nearly every day;
   (7) feelings of worthlessness or excessive or inappropriate guilt (which may be delusional) nearly every day (not merely self-reproach or guilt about being sick);
   (8) diminished ability to think or concentrate, or indecisiveness, nearly every day (either by their subjective account or as observed by others); and/or
   (9) recurrent thoughts of death (not just fear of dying), recurrent suicidal ideation without a specific plan, or a suicide attempt or a specific plan for committing suicide; and
(B) The symptoms cause clinically significant distress or impairment in social, occupational, or other important areas of functioning;
(C) The episode is not attributable to the direct physiological effects of a substance or to another medical condition.
(D) The occurrence of the major depressive episode is not better explained by schizoaffective disorder, schizophrenia, schizophreniform disorder, delusional disorder, or other specified and unspecified schizophrenia spectrum and other psychotic disorders; and
(E) There has never been a manic or hypomanic episode.

Major depressive episodes are further clinically distinguished by the presence (or absence) of any of the following additional features: (a) with anxious distress; (b) with mixed features; (c) with melancholic features; (d) with atypical features; (e) with psychotic features; (f) with catatonia; (g) with peripartum onset; and (h) with seasonal pattern.

Most patients with MDD have comorbid anxiety (54%-78%) according to the DSM-5 anxious distress specifier. Patients with MDD and anxious distress have worse psychosocial functioning and quality of life, increased suicide risk, and poorer treatment response compared to MDD patients without anxious distress.

Responses to a significant loss (e.g., bereavement, financial ruin, losses from a natural disaster, a serious medical illness or disability) may include the feelings of intense sadness, rumination about the loss, insomnia, poor appetite, and weight loss noted in Criterion A, which may resemble a depressive episode. However, although such symptoms may be understandable or considered appropriate to the loss, the presence of a major depressive episode in addition to the normal response to a significant loss should be considered. This decision inevitably requires the exercise of clinical judgement based on the individual's history and the cultural norms for the expression of distress in the context of loss.

Anhedonia occurs in about 70% of patients with major depressive disorder (MDD) and in about 50% of patients with bipolar depression. The DSM-5 definition of anhedonia is markedly diminished interest or pleasure. Heightened levels of anhedonia in mood disorders are associated with more severe and recurrent depressive illness, a greater risk for suicidal ideation, and poorer treatment response compared with lower levels of anhedonia. Anhedonia is a common residual symptom in patients treated for depression, and treatments that improve a wide range of anhedonia-related symptoms, including sadness and detachment, may improve outcomes.

Approximately 68% of MDD patients experience comorbid sexual dysfunction, and importantly, common antidepressant therapies often lead to new or worsened sexual dysfunction. The sexual dysfunction seen in MDD patients includes decreased sexual desire, decreased sexual excitement, and decreased orgasmic function. Each of these symptoms can have a strong negative impact on quality of life and, when associated with antidepressant therapy, is a leading cause of treatment nonadherence. Sexual dysfunction may also promote suicidality.

SSRI treatments are associated with a variety of sexual side effects. Among the most common are delayed ejaculation, reduced sexual desire, reduced sexual satisfaction, anorgasmia, and impotence (inability to obtain an erection in me, inadequate lubrication in women). Other symptoms include painful intercourse (dyspareunia), prolong erection (priapism), and genital numbness. It has been reported that sexual side effects are most common with paroxetine, followed by fluvoxamine, sertraline, fluoxetine, with escitalopram comparable to fluoxetine. One study reported a rate of sexual dysfunction of 75% for patients treated with paroxetine, versus 30% for patients treated with citalopram.

The sexual side effects associated with SSRI treatment are believed to be due directly to their mechanism of action, i.e., increasing serotonin availability at specific serotonin receptor subtypes, which results in changes in both dopamine levels and sex hormone levels. Serotonin/norepinephrine reuptake inhibitors (SNRIs), such as venlafaxine, desvenlafaxine, and duloxetine, tricyclic antidepressants, tetracyclic antidepressants, and monoamine oxidase inhibitors, have also been associated with these side effects, although at somewhat reduced levels compared to SSRIs. In many countries, all SSRIs and SNRIs carry warnings about sexual side effects. In 2024, Australian authorities updated the SSRI/SNRI labels to warn about the newly recognized risk of sexual side effects that outlasts the termination of SSRI/SNRI treatment (“persistent sexual dysfunction”). Recent evidence shows that these side effects can last from weeks up to 3-4 years following the cessation of antidepressant treatment. In one recent study, 62% of cases of persistent sexual dysfunction were associated with escitalopram, citalopram, paroxetine, sertraline, and fluoxetine.

As a result, patients experiencing severe sexual side effects made be switched to therapy with antidepressant agents that do not inhibit serotonin reuptake, such as bupropion (a dopamine/norepinephrine reuptake inhibitor) and mirtazapine (an alpha-2 antagonist and serotonin receptor antagonist)

Pharmaceutical treatments for MDD include a variety of drugs with a recommended multi-level treatment paradigm. Initial recommended treatment (Level 1) is monotherapy with a selective serotonin reuptake inhibitor (SSRI), serotonin-norepinephrine reuptake inhibitor (SNRI), vortioxetine, bupropion, or mirtazapine. If there is an inadequate (i.e., partial) response within 2-4 weeks, then treatment can be maintained for an additional 2 weeks. If there is no response at 4 weeks, then the patient is elevated to level 2 treatment. Level 2 treatment involves changing to a different monotherapy agent within the same or different class selected for Level 1, or combining the initial monotherapy agent with an adjunct agent. Suitable adjunct agents include second-generation antipsychotics (e.g., aripiprazole, brexpiprazole; quetiapine is not recommended), ketamine (racemic ketamine IV or esketamine intranasal), or a second antidepressant (but not combining two SSRIs or an SSRI and an SNRI). If patients remain inadequately treated under Level 2, then Level 3 treatment may involve the combination of an SSRI or SNRI with quetiapine, lithium, T3, L-methylfolate, or S-adenosylmethionine; or treatment with a tricyclic antidepressant (TVA), monoamine oxidase inhibitor (MAOI), or non-pharmacological interventions (electroconvulsive therapy or transcranial magnetic stimulation). A patient who is still not adequately treated may progress to Level 4, which may involve various triple drug combinations, or other non-pharmacological treatments (e.g., vagus nerve stimulation).

The above guidelines apply to MDD without any special features. Patients diagnosed with MDD with any of the above noted special features (anxious distress, mixed features, melancholic features, atypical features, psychotic features, catatonia, peripartum onset, seasonal pattern) have additional multi-level treatment guidelines.

Current treatments for MDD are often limited by delayed responses and undesirable side effects (e.g. weight gain, metabolic disturbances, sexual dysfunction, and disturbed sleep). In addition, after first-line antidepressant treatment (ADT), about 75% of MDD patients fail to achieve remission, and about 60% of MDD patients fail to achieve response. This necessitates switching of the patient to an alternative ADT or adding an adjunctive agent. Considering that first-line antidepressant therapy (e.g., SSRI therapy) typically takes several weeks of treatment to demonstrate efficacy, if initial treatment fails, switching or adjunctive treatment (e.g., antipsychotic therapy) causes a considerable further delay in achieving a successful patient outcome. As a result, inadequate response to ADT is associated with increased hospitalization and suicide risk, and greater impairments in patient functioning.

It is apparent that many patients fail to respond to existing treatments for MDD and there is an urgent need for new treatments for this debilitating disease, especially for the treatment of anhedonia in MDD patients, and for the reduction of comorbid sexual dysfunction in MDD patients.

### BRIEF SUMMARY

The present disclosure provides methods for the treatment of major depressive disorder (MDD), comprising the administration of pharmaceutical capsules comprising 42 mg of lumateperone (60 mg of lumateperone tosylate) to a patient in need thereof. The present disclosure further provides the method for treating such patients with moderate to severe hepatic impairment or concomitant with a moderate CYP3A4 inhibitor, using a dosage of 21 mg of lumateperone (30 mg of lumateperone tosylate), or 10.5 mg of lumateperone (15 mg of lumateperone tosylate) for a strong CYP 3A4 inhibitor. In some embodiments, the method is adjunct to administration of antidepressant therapy (e.g., SSRI, SNRI, or bupropion). The present disclosure further provides methods of treating MDD in patients suffering from anxious distress, in patients suffering from anhedonia, in patients exhibiting MDD with mixed features, and/or in patients suffering from sexual dysfunction.

Further embodiments will be apparent from the following detailed description and examples.

### BRIEF DESCRIPTION OF THE DRAWINGS

FIG. 1: Effects of Intrinsic Factors on Lumateperone Pharmacokinetics. This graph shows the changes (fold change and 90% confidence interval) in Cₘₐₓ (maximum plasma concentration) and AUCt (area under the plasma concentration vs. time curve) in patients with hepatic impairment or renal impairment.

FIG. 2: Effects of Other Drugs on Lumateperone Pharmacokinetics. This graph shoes the changes (fold change and 90% confidence interval) in Cₘₐₓ (maximum plasma concentration) and AUCt (area under the plasma concentration vs. time curve) in patients receiving concomitant treatment with itraconazole, diltiazem, rifampin, probenecid, and valproic acid.

FIG. 3A: Change from Baseline in MADRS Total Score by Time (Weeks) in Patients with Major Depressive Disorder (MDD) (Study 5). CAPLYTA 42 mg refers to a dosage of lumateperone tosylate of 60 mg.

FIG. 3B: LS Mean Change from Baseline to Day 43 in MADRS Single Items (Study 5) (MMRM in the mITT population; **P<0.01, ***P<0.001, ****P<0.0001, LSMD vs. placebo). Lumateperone 42 mg refers to lumateperone free base, equivalent to 60 mg lumateperone tosylate.

FIG. 3C: LS Mean Change from Baseline to Day 43 in MADRS Single Items (Study 6) (MMRM in the mITT population; *P<0.05, **P<0.01, ***P<0.001, ****P<0.0001, LSMD vs. placebo). Lumateperone 42 mg refers to lumateperone free base, equivalent to 60 mg lumateperone tosylate.

FIG. 4: Study 5 and 6, Pooled Results: LS Mean Change from Baseline in MADRS Total Score (mixed-effects model for repeated measures (MMRM) in the mITT population; ****P<0.0001, LSMD vs. placebo). Lumateperone 42 mg refers to lumateperone free base, equivalent to 60 mg lumateperone tosylate.

FIG. 5: Study 5 and 6, Pooled Results: LS Mean Change from Baseline in CGI-S Score (MMRM in the mITT population; **P<0.01, ***P<0.001, ****P<0.0001, LSMD vs. placebo). Lumateperone 42 mg refers to lumateperone free base, equivalent to 60 mg lumateperone tosylate.

FIG. 6: Study 5 and 6, Pooled Results: MADRS Response and Remission Rates at Day 43 (mITT population; ****P<0.0001, vs. placebo). Lumateperone 42 mg refers to lumateperone free base, equivalent to 60 mg lumateperone tosylate (plus ADT in both groups).

FIG. 7: Study 5 and 6, Pooled Results: LS Mean Change from Baseline to Day 43 in QIDS-SR-16 Total Score (ANCOVA in ITT population; ****P<0.0001, vs. placebo). Lumateperone 42 mg refers to lumateperone free base, equivalent to 60 mg lumateperone tosylate.

FIG. 8: Mean Change from Baseline in MADRS Total Score through open-label extension period (EOLTP, end of long-term treatment period; OLE, open-label extension; Lumateperone 42 mg refers to lumateperone free base, equivalent to 60 mg lumateperone tosylate).

FIG. 9: LS Mean Change from Baseline in MADRS Total Score in Patients with Anxious Distress (Study 5, Post-Hoc Analysis; MMRM in the mITT population; **P<0.01, ****P<0.0001, LSMD vs. placebo). Lumateperone 42 mg refers to a dosage of lumateperone tosylate of 60 mg.

FIG. 10: LS Mean Change from Baseline in CGI-S Score in Patients with Anxious Distress (Study 5, Post-Hoc Analysis; MMRM in the mITT population; ***P<0.001, ****P<0.0001, LSMD vs. placebo). Lumateperone 42 mg refers to a dosage of lumateperone tosylate of 60 mg.

FIG. 11: LS Mean Change from Baseline in MADRS Inner Tension Item Score in Patients with Anxious Distress (Study 5, Post-Hoc Analysis; (MMRM in the mITT population; *P<0.05, **P<0.01, ****P<0.0001, LSMD vs. placebo). Lumateperone 42 mg refers to a dosage of lumateperone tosylate of 60 mg.

FIG. 12 (A and B): LS Mean Change from Baseline to Day 43 in QIDS-SR-16 (A) Total Score GAD-7 Total Score (B) in Patients with Anxious Distress (Study 5, Post-Hoc Analysis; ANCOVA-LOCF in the ITT population; ****P<0.0001, LSMD vs. placebo). Lumateperone 42 mg refers to a dosage of lumateperone tosylate of 60 mg.

FIG. 13: LS Mean Change from Baseline in MADRS Anhedonia Factor Score in Study 5 (**P<0.01, ****P<0.0001, LSMD vs. placebo, MMRM in mITT population). Lumateperone 42 mg refers to a dosage of lumateperone tosylate of 60 mg.

FIG. 14 (A and B): (A) LS Mean Change from Baseline in MADRS Total Score in Study 5 in Patients with Anhedonia Baseline Higher than Median; (B) LS Mean Change from Baseline in MADRS Anhedonia Factor Score in Study 5 in Patients with Anhedonia Baseline Higher than Median (*P<0.05, **P<0.01, ****P<0.0001, LSMD vs. placebo MMRM. Lumateperone 42 mg refers to a dosage of lumateperone tosylate of 60 mg.

FIG. 15 (A and B): LS Mean Change from Baseline in MADRS Total Score at Day 43 among (A) demographic subgroups or (B) disease characteristic subgroups in Study 5.

FIG. 16 (A and B): LS Mean Change from Baseline in CGI-S Score at Day 43 among (A) demographic subgroups or (B) disease characteristic subgroups in Study 5.

FIG. 17 (A and B): LS Mean Change from Baseline in QIDS-SR-16 Total Score at Day 43 among (A) demographic subgroups or (B) disease characteristic subgroups in Study 5.

FIG. 18: Pooled Analysis of First Onset of Common TEAEs in Study 5 and Study 6.

FIG. 19: Pooled Analysis of Duration of Common TEAEs in Study 5 and Study 6.

FIG. 20: Pooled Analysis of MADRS Total Score Remission Rate at Day 43 in Study 5 and 6 in Patient Subgroups.

FIG. 21: MADRS Total Score Remission Rate at Week 26 of Open-Label Extension Study in Patient Subgroups.

FIG. 22 (A and B): MADRS Total score Sustained Remission Rate in (A) Pooled Study 5 and Study 6 Results and (B) During Open-Label Extension Period.

FIG. 23 (A and B): (A) LS Mean Change from Baseline in MADRS Anhedonia Factor Score in Study 7 Combined MDD/BPD Population; (B) LS Mean Change from Baseline in MADRS Anhedonia Factor Items in Study 7 Combined MDD/BPD Population (**P<0.01, ****P<0.0001, LSMD vs. placebo MMRM. Lumateperone 42 mg refers to a dosage of lumateperone tosylate of 60 mg.

FIG. 24 (A and B): (A) LS Mean Change from Baseline in MADRS Anhedonia Factor Score in Study 7 MDD Population; (B) LS Mean Change from Baseline in MADRS Anhedonia Factor Items in Study 7 MDD Population (*P<0.05, **P<0.01, ***P<0.01, ****P<0.0001, LSMD vs. placebo MMRM). Lumateperone 42 mg refers to a dosage of lumateperone tosylate of 60 mg.

FIG. 25: LS Mean Change from Baseline CSFQ-14 Total Score in Study 6 ITT Population (****P<0.0001, LSMD vs. placebo MMRM). Lumateperone 42 mg refers to a dosage of lumateperone tosylate of 60 mg.

FIG. 26: LS Mean Change from Baseline CSFQ-14 Total Score in Study 6 Patients with Baseline Sexual Dysfunction (****P<0.0001, LSMD vs. placebo MMRM). Lumateperone 42 mg refers to a dosage of lumateperone tosylate of 60 mg.

FIG. 27: LS Mean Change from Baseline in CSFQ-14 Subscale Scores in ITT Population (*P<0.05, **P<0.01, ***P<0.01, ****P<0.0001, LSMD vs. placebo MMRM). Lumateperone 42 mg refers to a dosage of lumateperone tosylate of 60 mg.

### DETAILED DESCRIPTION

Lumateperone (administered as lumateperone tosylate) is an atypical antipsychotic indicated for the treatment of schizophrenia and bipolar depression in adults. Lumateperone is being evaluated for approval for the treatment of major depressive disorder (MDD), such as, adjunctive to antidepressant therapy.

Lumateperone currently has regulatory approval in the United States only for the lumateperone tosylate salt form (lumateperone monotosylate). The recommended dose for this drug is 42 mg of lumateperone administered orally once daily, which corresponds to 60 mg of lumateperone tosylate. However, in patients co-administered strong CYP 3A4 inhibitors, the recommended dose is 10.5 mg of lumateperone daily (15 mg of lumateperone tosylate), while for patients co-administered moderate CYP3A4 inhibitors, the recommend dose is 21 mg of lumateperone daily (30 mg of lumateperone tosylate). In patients having moderate or severe hepatic impairment, the recommend dose is 21 mg of lumateperone daily (30 mg of lumateperone tosylate). Lumateperone is taken orally as a capsule, optionally with food. Unlike other drugs of the atypical antipsychotic class, dose titration is not required.

In a first aspect, the present disclosure provides a method (Method 1) for the treatment of major depressive disorder (MDD), comprising the administration of pharmaceutical capsules comprising 42 mg of lumateperone (60 mg of lumateperone tosylate) once daily to a patient in need thereof, provided that if the patient has moderate to severe hepatic impairment or treatment is concomitant with a moderate CYP 3A4 inhibitor, the patient is administered pharmaceutical capsules comprising 21 mg of lumateperone (30 mg of lumateperone tosylate) once daily, and if treatment is concomitant with a strong CYP 3A4 inhibitor, then the patient is administered pharmaceutical capsules comprising 10.5 mg of lumateperone (15 mg of lumateperone tosylate) once daily. Optionally, the method is adjunctive to therapy with antidepressants (e.g., SSRIs, SNRIs, or bupropion). Optionally, the patient is an adult.

In further embodiments of the first aspect, the present disclosure provides:
1.1. Method 1, wherein the patient has no hepatic impairment or mild hepatic impairment (e.g., Child-Pugh Class A), and/or wherein the patient is not concomitantly administered a CYP 3A4 inhibitor, and the patient is administered 42 mg of lumateperone (60 mg of lumateperone tosylate) daily;
1.2. Method 1.2, wherein the oral daily dose of 42 mg lumateperone (60 mg lumateperone tosylate) is provided as a single capsule, optionally wherein the oral daily dose is taken with food;
1.3. Method 1.1 or 1.2, wherein the capsule has a blue cap and opaque white body;
1.4. Method 1, wherein the patient has or is diagnosed with moderate to severe hepatic impairment (e.g., Child-Pugh Class B or C), and the patient is administered 21 mg of lumateperone (30 mg of lumateperone tosylate) daily;
1.5. Method 1.4, wherein the oral daily dose of 21 mg lumateperone (30 mg lumateperone tosylate) is provided as a single capsule, optionally wherein the oral daily dose is taken with food;
1.6. Method 1.4 or 1.5, wherein the capsule has an opaque white cap and body;
1.7. Method 1, wherein the patient is concomitantly administered a moderate CYP 3A4 inhibitor, and the patient is administered 21 mg of lumateperone (30 mg of lumateperone tosylate) daily;
1.8. Method 1.7, wherein the moderate CYP 3A4 inhibitor is selected from amprenavir, ciprofloxacin, cyclosporine, diltiazem, erythromycin, fluconazole, fluvoxamine, and verapamil;
1.9. Method 1.7 or 1.8, wherein the oral daily dose of 21 mg lumateperone (30 mg lumateperone tosylate) is provided as a single capsule, optionally wherein the oral daily dose is taken with food;
1.10. Method 1.7, 1.8, or 1.9, wherein the capsule has an opaque white cap and body;
1.11. Method 1, wherein the patient is concomitantly administered a strong CYP 3A4 inhibitor, and the patient is administered 10.5 mg of lumateperone (15 mg of lumateperone tosylate) daily;
1.12. Method 1.11, wherein the strong CYP3A4 inhibitor is selected from clarithromycin, grapefruit juice, itraconazole, voriconazole, nefazodone, ritonavir, and nelfinavir;
1.13. Method 1.11 or 1.12, wherein the oral daily dose of 10.5 mg lumateperone (15 mg lumateperone tosylate) is provided as a single capsule, optionally wherein the oral daily dose is taken with food;
1.14. Method 1.11, 1.12, or 1.13, wherein the capsule has an opaque light pink cap and body;
1.15. Method 1, or any of 1.1-1.14, wherein the capsule is supplied in a blister pack or in a box (e.g., a box of 30). 1.16. Any of Methods 1.1-1.15, wherein the capsule further comprises croscarmellose sodium, gelatin, magnesium stearate, mannitol, talc, and colorants (e.g., titanium dioxide, FD&C Blue #1, and/or FD&C Red #3);
1.17. Method 1.16, wherein the capsule consists of lumateperone tosylate, croscarmellose sodium, gelatin, magnesium stearate, mannitol, talc, and colorants (e.g., titanium dioxide, FD&C Blue #1, and/or FD&C Red #3);
1.18. Method 1, or any of 1.1-1.17, wherein the patient is not under concomitant treatment with a CYP3A4 inducer (e.g., carbamazepine, phenytoin, rifampin, St. John's wort, bosentan, efavirenz, etravirine, modafinil, nafcillin, aprepitant, armodafinil, pioglitazone, prednisone);
1.19. Method 1, or any of 1.1-1.18, wherein the patient is not under concomitant treatment with a UGT inhibitor (e.g., valproic acid, probenecid);
1.20. Method 1, or any of 1.1-1.18, wherein the patient is under concomitant treatment with a CYP3A4 inducer and/or a UGT inhibitor;
1.21. Method 1, or any of 1.1-1.20, wherein the patient has previously been treated with another therapeutic agent for the treatment of MDD, but such treatment was terminated due to a need to undergo concomitant treatment with, or due to side effects resulting from concomitant treatment with, a CYP3A4 inducer, a moderate or strong CYP3A4 inhibitor, and/or a UGT inhibitor;
1.22. Method 1, or any of 1.1-1.21, wherein the patient is under concomitant treatment with a monoamine oxidase inhibitor (MAOI);
1.23. Method 1, or any of 1.1-1.21, wherein the patient has been under treatment with a monoamine oxidase inhibitor (MAOI) within the previous 14-days prior to initiation of treatment with lumateperone, or wherein MAOI treatment is initiated while the patient is undergoing lumateperone treatment;
1.24. Method 1, or any of 1.1-1.20, wherein the patient has previously been treated with another therapeutic agent for the treatment of MDD, but such treatment was terminated due to a need to undergo concomitant treatment with, or due to side effects resulting from concomitant treatment with, an MAOI;
1.25. Method 1.22, 1.23, or 1.24, wherein the MAOI is selected from isocarboxazid, hydracarbazine, phenelzine, tranylcypromine, bifemelane, methylene blue, moclobemide, pirlindole, rasagiline, selegiline, safinamide, safrazine, linezolid, furazolidone, brofaromine, caroxazone, eprobemide, metralindole, and minaprine;
1.26. Method 1, or any of 1.1-1.25, wherein the patient is less than 65 years of age and/or does not have, or does not have a history of, dementia-related psychosis, optionally wherein the patient is at least 18 years of age;
1.27. Method 1, or any of 1.1-1.25, wherein the patient has, or has a history of, dementia-related psychosis;
1.28. Method 1, or any of 1.1-1.27, wherein the patient is less than 18 years of age;
1.29. Method 1, or any of 1.1-1.27, wherein the patient is at least 65 years of age and/or has, or has a history of, dementia-related psychosis;
1.30. Method 1.29, wherein the patient has previously been treated with another therapeutic agent for the treatment of MDD, but such treatment was terminated due to the development of, or the risk of development of, dementia-related psychosis;
1.31. Method 1, or any of 1.1-1.30, wherein the patient does not have, or is not at risk of developing, tardive dyskinesia;
1.32. Method 1, or any of 1.1-1.30, wherein the patient has, or is at risk of developing, tardive dyskinesia;
1.33. Method 1.32, wherein the patient has previously been treated with another therapeutic agent for the treatment of MDD, but such treatment was terminated due to the development of, or the risk of development of, tardive dyskinesia;
1.34. Method 1, or any of 1.1-1.33, wherein the patient does not have, or is not at risk of developing, hyperglycemia or diabetes mellitus (e.g., the patient does not have a history of hyperglycemia or diabetes mellitus);
1.35. Method 1, or any of 1.1-1.33, wherein the patient has, or is at risk of developing, hyperglycemia or diabetes mellitus (e.g., the patient has a history of hyperglycemia or diabetes mellitus);
1.36. Method 1.35, wherein the patient has previously been treated with another therapeutic agent for the treatment of MDD, but such treatment was terminated due to the development of, or the risk of development of, hyperglycemia or diabetes mellitus;
1.37. Method 1, or any of 1.1-1.36, wherein the patient does not have, or is not at risk of developing, dyslipidemia (e.g., the patient does not have a history of dyslipidemia). 1.38. Method 1, or any of 1.1-1.36, wherein the patient has, or is at risk of developing, dyslipidemia (e.g., the patient has a history of dyslipidemia);
1.39. Method 1.38, wherein the patient has previously been treated with another therapeutic agent for the treatment of MDD but such treatment was terminated due to the development of, or the risk of development of, dyslipidemia;
1.40. Method 1, or any of 1.1-1.39, wherein the patient does not have, or is not at risk of developing, weight gain (e.g., the patient does not have a history of difficulty controlling weight);
1.41. Method 1, or any of 1.1-1.39, wherein the patient has, or is at risk of developing, weight gain (e.g., the patient has a history of difficulty controlling weight);
1.42. Method 1.41, wherein the patient has previously been treated with another therapeutic agent for the treatment of MDD, but such treatment was terminated due to the development of, or the risk of development of, weight gain;
1.43. Method 1, or any of 1.1-1.42, wherein the patient does not have, or is not at risk of developing, leukopenia, neutropenia and/or agranulocytosis (e.g., as determined by CBC testing, including WBC and ANC);
1.44. Method 1, or any of 1.1-1.43, wherein the patient has, or is at risk of developing, leukopenia, neutropenia and/or agranulocytosis (e.g., as determined by CBC testing, including WBC and ANC);
1.45. Method 1.44, wherein the patient has previously been treated with another therapeutic agent for the treatment of MDD, but such treatment was terminated due to the development of, or the risk of development of, leukopenia, neutropenia and/or agranulocytosis (e.g., as determined by CBC testing, including WBC and ANC);
1.46. Method 1, or any of 1.1-1.45, wherein the patient does not have, or is not at risk of developing, orthostatic hypertension or syncope (e.g., the patient does not have a history of orthostatic hypertension or syncope);
1.47. Method 1, or any of 1.1-1.45, wherein the patient has, or is at risk of developing, orthostatic hypertension or syncope (e.g., the patient has a history of orthostatic hypertension or syncope);
1.48. Method 1.47, wherein the patient has previously been treated with another therapeutic agent for the treatment of MDD, but such treatment was terminated due to the development of, or the risk of development of, orthostatic hypertension or syncope;
1.49. Method 1, or any of 1.1-1.48, wherein the patient does not have, or is not at risk of developing, seizures (e.g., the patient does not have a history of seizures or a lowered seizure threshold);
1.50. Method 1, or any of 1.1-1.48, wherein the patient has, or is at risk of developing, seizures (e.g., the patient has a history of seizures or a lowered seizure threshold);
1.51. Method 1.50, wherein the patient has previously been treated with another therapeutic agent for the treatment of MDD, but such treatment was terminated due to the development of, or the risk of development of, seizures or a lowered seizure threshold. 1.52. Method 1, or any of 1.1-1.51, wherein the patient does not have, or is not at risk of developing, cognitive or motor impairment (e.g., the patient does not have a history of cognitive or motor impairment);
1.53. Method 1, or any of 1.1-1.51, wherein the patient has, or is at risk of developing, cognitive or motor impairment (e.g., the patient has a history of cognitive or motor impairment);
1.54. Method 1.53, wherein the patient has previously been treated with another therapeutic agent for the treatment of MDD, but such treatment was terminated due to the development of, or the risk of development of, cognitive or motor impairment;
1.55. Method 1, or any of 1.1-1.54, wherein the method does not cause cognitive or motor impairment;
1.56. Method 1, or any of 1.1-1.55, wherein the patient does not have, or is not at risk of developing, body temperature dysregulation (e.g., the patient does not have a history of body temperature dysregulation, or the patient does not concomitantly engage in strenuous exercise, or is not exposed to extreme heat or dehydration, or does not concomitantly take anticholinergic medications);
1.57. Method 1, or any of 1.1-1.55, wherein the patient has, or is at risk of developing, body temperature dysregulation (e.g., the patient has a history of body temperature dysregulation, or the patient concomitantly engages in strenuous exercise, or is exposed to extreme heat or dehydration, or concomitantly takes anticholinergic medications);
1.58. Method 1.57, wherein the patient has previously been treated with another therapeutic agent for the treatment of MDD, but such treatment was terminated due to the development of, or the risk of development of, body temperature dysregulation. 1.59. Method 1, or any of 1.1-1.58, wherein the patient is switching from treatment with another therapeutic agent for the treatment of MDD, to treatment with lumateperone (e.g., lumateperone tosylate), wherein the switch does not include dose titration;
1.60. Method 1, or any of 1.1-1.59, wherein the method does not cause serotonin syndrome;
1.61. Method 1, or any of 1.1-1.60, wherein the patient has, or is at risk of developing, serotonin syndrome;
1.62. Method 1, or any of 1.1-1.61, wherein has previously been treated with another therapeutic agent for the treatment of MDD, but such treatment was terminated due to the development of, or the risk of development of, serotonin syndrome;
1.63. Method 1, or any of 1.1-1.62, wherein the method does not cause hyponatremia (e.g., symptomatic hyponatremia);
1.64. Method 1, or any of 1.1-1.63, wherein the patient has, or is at risk of developing, hyponatremia (e.g., symptomatic hyponatremia);
1.65. Method 1, or any of 1.1-1.64, wherein has previously been treated with another therapeutic agent for the treatment of MDD, but such treatment was terminated due to the development of, or the risk of development of, hyponatremia (e.g., symptomatic hyponatremia);
1.66. Method 1, or any of 1.1-1.65, wherein the method does not cause an increase in risk of bleeding;
1.67. Method 1, or any of 1.1-1.66, wherein the patient has, or is at risk of developing, bleeding or a bleeding disorder or blood-clotting disorder (e.g., hemophilia, Von Willebrand disease, vitamin K deficiency, disseminated intravascular coagulation, thrombocytopenia, uremia, congenital afibrinogenemia, Factor V deficiency, Factor X deficiency Factor XI deficiency);
1.68. Method 1, or any of 1.1-1.67, wherein has previously been treated with another therapeutic agent for the treatment of MDD, but such treatment was terminated due to the development of, or the risk of development of, bleeding or a bleeding disorder or blood-clotting disorder (e.g., ulcer (e.g., gastric or duodenal peptic ulcer), leukemia, hemophilia, Von Willebrand's disease, vitamin K deficiency, disseminated intravascular coagulation, thrombocytopenia, uremia, congenital afibrinogenemia, Factor V deficiency, Factor X deficiency Factor XI deficiency, chronic liver diseases;
1.69. Method 1, or any of 1.1-1.68, wherein the patient is currently, or has recently (e.g., in the last 3 days, 5 days, 7 days, 14 days, or 30 days), been treated with a therapeutic agent which increases the risk of bleeding (e.g., an anticoagulant or an NSAID);
1.70. Method 1, or any of 1.1-1.69, wherein has previously been treated with another therapeutic agent for the treatment of MDD, but such treatment was terminated due to the need to undergo treatment with a therapeutic agent which increases the risk of bleeding (e.g., an anticoagulant or an NSAID);
1.71. Method 1.69 or 1.70, wherein said therapeutic agent which increases the risk of bleeding is selected from aspirin, warfarin, heparin, coumarin, dabigatran, dabigatran etexilate, rivaroxaban, apixaban, edoxaban, betrixaban, ibuprofen, diclofenac, and naproxen;
1.72. Method 1, or any of 1.1-1.71, wherein the method does not cause glaucoma (e.g., angle-closure glaucoma);
1.73. Method 1, or any of 1.1-1.72, wherein the patient has, or is at risk of developing, glaucoma (e.g., angle-closure glaucoma), e.g., wherein the patient has untreated anatomically narrow angles;
1.74. Method 1, or any of 1.1-1.73, wherein has previously been treated with another therapeutic agent for the treatment of MDD, but such treatment was terminated due to the development of, or the risk of development of, glaucoma (e.g., angle-closure glaucoma);
1.75. Method 1, or any of 1.1-1.74, wherein the method does not cause sexual dysfunction (e.g., disorders of sexual drive, sexual arousal, orgasm, and ejaculation; painful intercourse, prolonged erection, or genital numbness);
1.76. Method 1, or any of 1.1-1.75, wherein the patient has, or is at risk of developing, sexual dysfunction (e.g., disorders of sexual drive, sexual arousal, orgasm, and ejaculation; painful intercourse, prolonged erection, or genital numbness);
1.77. Method 1, or any of 1.1-1.76, wherein has previously been treated with another therapeutic agent for the treatment of MDD, but such treatment was terminated due to the development of, or the risk of development of, sexual dysfunction (e.g., disorders of sexual drive, sexual arousal, orgasm, and ejaculation; painful intercourse, prolonged erection, or genital numbness);
1.78. Method 1, or any of 1.1-1.77, wherein the patient is concurrently treated with pimozide or thioridazine;
1.79. Any preceding method, wherein the MDD is characterized by major depressive episodes;
1.80. Any preceding method, wherein the MDD is characterized by major depressive episodes with anxious distress;
1.81. Any preceding method, wherein the MDD is characterized by major depressive episodes with mixed features;
1.82. Any preceding method, wherein the MDD is characterized by major depressive episodes with melancholic features;
1.83. Any preceding method, wherein the MDD is characterized by major depressive episodes with atypical features;
1.84. Any preceding method, wherein the MDD is characterized by major depressive episodes with psychotic features;
1.85. Any preceding method, wherein the MDD is characterized by major depressive episodes with catatonia;
1.86. Any preceding method, wherein the MDD is characterized by major depressive episodes with peripartum onset;
1.87. Any preceding method, wherein the MDD is characterized by major depressive episodes with seasonal pattern;
1.88. Any preceding method, wherein the patient has been previously treated with an SSRI, such as citalopram, escitalopram, fluoxetine, fluvoxamine, paroxetine, or sertraline, and the patient's symptoms did not adequately improve (e.g., the treatment was ineffective or not adequately effective);
1.89. Any preceding method, wherein the patient has been previously treated with an SNRI, such as desvenlafaxine, duloxetine, levomilnacipran, milnacipran, tofenacin, or venlafaxine, and the patient's symptoms did not adequately improve (e.g., the treatment was ineffective or not adequately effective);
1.90. Any preceding method, wherein the patient has been previously treated with vortioxetine, bupropion, or mirtazapine, and the patient's symptoms did not adequately improve (e.g., the treatment was ineffective or not adequately effective);
1.91. Any preceding method, wherein the patient has been previously treated with a TCA, such as amitriptyline, amitriptyline oxide, clomipramine, desipramine, dibenzepin, dimetacrine, dosulepin, doxepin, imipramine, lofepramine, melitracen, melixeran, nitroxazepine, nortriptyline, noxiptiline, pipofezine, protriptyline, or trimipramine, and the patient's symptoms did not adequately improve (e.g., the treatment was ineffective or not adequately effective);
1.92. Any preceding method, wherein the patient has been previously treated with an antidepressant agent in combination with an antipsychotic, such as haloperidol, aripiprazole, quetiapine, olanzapine, risperidone, lurasidone, paliperidone, iloperidone, ziprasidone, brexpiprazole, asenapine, clozapine, or zotepine, and the patient's symptoms did not adequately improve (e.g., the treatment was ineffective or not adequately effective);
1.93. Any preceding method, wherein the patient has been previously treated with an antidepressant agent in combination with an atypical antipsychotic, such as risperidone, aripiprazole, olanzapine, quetiapine, or ziprasidone, and the patient's symptoms did not adequately improve (e.g., the treatment was ineffective or not adequately effective);
1.94. Any preceding method, wherein the method does not result in the patient developing tardive dyskinesia;
1.95. Any preceding method, wherein the method does not result in the patient developing hyperglycemia or diabetes mellitus (e.g., as shown by monitoring of fasting glucose levels, insulin levels, and/or hemoglobin A1c levels in plasma);
1.96. Any preceding method, wherein the method does not result in the patient developing dyslipidemia (e.g., as shown by monitoring of plasma lipid panels, including cholesterol, triglycerides, VLDL, LDL and HDL levels);
1.97. Any preceding method, wherein the method does not result in the patient developing weight gain (e.g., as compared to the patient's weight before treatment and/or with reference to the normal body-mass index for a person of said patient's height);
1.98. Any preceding method, wherein the method does not result in the patient developing orthostatic hypertension or syncope;
1.99. Any preceding method, wherein the method further comprises the step of evaluating the patient, prior to the initiation of treatment with lumateperone (e.g., lumateperone tosylate), for signs of cerebrovascular disease, cardiovascular disease, neuroleptic malignant syndrome, tardive dyskinesia, hyperglycemia, diabetes mellitus, dyslipidemia, weight gain, and/or orthostatic vital signs;
1.100. Any preceding method, wherein the method further comprises the step of evaluating the patient, periodically during treatment with lumateperone tosylate, for signs of cerebrovascular disease, cardiovascular disease, neuroleptic malignant syndrome, tardive dyskinesia, hyperglycemia, diabetes mellitus, dyslipidemia, weight gain, and/or orthostatic vital signs (e.g., every 1 month, 2 months, 3 months, 4 months, 6 months, 9 months, 12 months, 18 months or 24 months);
1.101. Any preceding method, wherein the method provides improved sleep (e.g., increased somnolence and/or sedation);
1.102. Any preceding method, wherein the administration of the lumateperone is adjunct to treatment with an antidepressant agent, e.g., an SSRI, SNRI, vortioxetine, bupropion, or mirtazapine;
1.103. Method 1.102, wherein the SSRI is selected from citalopram, escitalopram, fluoxetine, fluvoxamine, paroxetine, and sertraline;
1.104. Method 1.102, wherein the SNRI is selected from desvenlafaxine, duloxetine, levomilnacipran, milnacipran, tofenacin, and venlafaxine;
1.105. Any preceding method, wherein the method provides a significant reduction in the MADRS total score within 43 days of the beginning of treatment, e.g., at least a 9-point reduction on the MADRS total score, or at least a 10-point reduction, or at least a 12-point reduction, or at least a 14-point reduction, or at least a 15-point reduction, or at least a 16-point reduction, or at least an 18-point reduction;
1.106. Any preceding method, wherein the method provides a significant reduction in the MADRS total score within 22 days of the beginning of treatment, e.g., at least a 4-point reduction on the MADRS total score, or at least a 5-point reduction, or at least a 6-point reduction, or at least an 8-point reduction, or at least a 10-point reduction;
1.107. Any preceding method, wherein the method provides a significant reduction in the MADRS total score within 15 days of the beginning of treatment, e.g., at least a 4-point reduction on the MADRS total score, or at least a 5-point reduction, or at least a 6-point reduction, or at least a 7-point reduction, or at least an 8-point reduction;
1.108. Any preceding method, wherein the method provides a significant reduction in the MADRS total score within 8 days of the beginning of treatment, e.g., at least a 3-point reduction on the MADRS total score, or at least a 4-point reduction, or at least a 5-point reduction;
1.109. Any preceding method, wherein the method provides a significant reduction in the CGI-S total score within 43 days of the beginning of treatment, e.g., within 22 days, or within 15 days, or within 8 days;
1.110. Any preceding method, wherein the patient is diagnosed with MDD according to the DSM-5-TR criteria, or according to DSM-IV-TR criteria, or according to the DSM-5 criteria;
1.111. Any preceding method, wherein the patient had an inadequate response to previous antidepressant therapy (e.g., one or two courses of prior antidepressant therapy), e.g., antidepressant therapy of at least 6 weeks duration (e.g., each course);
1.112. Method 1.111, wherein the previous antidepressant therapy (e.g., each course) is selected from an SSRI, SNRI, vortioxetine, bupropion, or mirtazapine, e.g., an SSRI, SNRI, or bupropion;
1.113. Method 1.112, wherein the SSRI is selected from citalopram, escitalopram, fluoxetine, fluvoxamine, paroxetine, and sertraline;
1.114. Method 1.112, wherein the SNRI is selected from desvenlafaxine, duloxetine, levomilnacipran, milnacipran, tofenacin, and venlafaxine;
1.115. Any one of Methods 1.111-1.114, wherein the inadequate response is characterized by less than a 50% improvement in symptoms (e.g., as measured by MADRS total score and/or CGI-S score and/or by patient questionnaire evaluation);
1.116. Any preceding method, wherein the patient has experienced a major depressive episode characterized by an MADRS total score of greater than or equal to 24 (e.g., greater than 30) and/or a CGI-S score of greater than or equal to 4 (e.g., greater than 4.5);
1.117. Method 1.116, wherein the major depressive episode is further characterized by a Quick Inventory of Depressive Symptomology-Self Report-16 Item (QIDS-SR-16) score of greater than or equal to 14 (e.g., greater than 17);
1.118. Any preceding method, wherein the MDD is characterized by major depressive episodes characterized by an MADRS total score of greater than or equal to 24 (e.g., greater than 30) and/or a CGI-S score of greater than or equal to 4 (e.g., greater than 4.5);
1.119. Method 1.118, wherein the major depressive episodes are further characterized by a Quick Inventory of Depressive Symptomology-Self Report-16 Item (QIDS-SR-16) score of greater than or equal to 14 (e.g., greater than 17);
1.120. Any preceding method, wherein the method treats a patient experiencing a major depressive episode characterized by an MADRS total score of greater than or equal to 24 (e.g., greater than 30) and/or a CGI-S score of greater than or equal to 4 (e.g., greater than 4.5);
1.121. Method 1.120, wherein the major depressive episode is further characterized by a Quick Inventory of Depressive Symptomology-Self Report-16 Item (QIDS-SR-16) score of greater than or equal to 14 (e.g., greater than 17);
1.122. Any preceding method, wherein the patient suffers from an anxiety disorder (e.g., generalized anxiety disorder) or wherein the patient suffers from MDD with anxious distress, wherein the generalized anxiety disorder or anxious distress is characterized by GAD-7 total score of greater than or equal to 10, and/or wherein the patient experiences 2 or more anxious symptoms (e.g., feeling tense, feeling restless, difficulty concentrating because of worry, fearful something awful may happen, or feeling out of control) during the majority of days during a major depressive episode;
1.123. Method 1.122, wherein the method provides a significant reduction in the MADRS total score within 8 days of the beginning of treatment, e.g., at least a 2-point reduction on the MADRS total score, or at least a 3-point reduction, or about a 4-point reduction;
1.124. Method 1.122 or 1.123, wherein the method provides a significant reduction in the MADRS total score within 15 days of the beginning of treatment, e.g., at least a 4-point reduction on the MADRS total score, or at least a 5-point reduction, or at least a 6-point reduction, or at least a 7-point reduction, or about an 8-point reduction;
1.125. Method 1.122, 1.123, or 1.124, wherein the method provides a significant reduction in the MADRS total score within 22 days of the beginning of treatment, e.g., at least an 8-point reduction on the MADRS total score, or at least a 9-point reduction, or about a 10-point reduction;
1.126. Method 1.122, 1.123, 1.124, or 1.125, wherein the method provides a significant reduction in the MADRS total score within 43 days of the beginning of treatment, e.g., at least a 10-point reduction on the MADRS total score, or at least a 12-point reduction, or at least a 14-point reduction, or about a 16-point reduction;
1.127. Any of Methods 1.122-1.126, wherein the method provides a significant reduction in the CGI-S score within 22 days of the beginning of treatment, e.g., at least a 0.5-point reduction of the CGI-S score, or at least a 0.7-point reduction, or at least a 0.8-point reduction, or about a 0.9-point reduction;
1.128. Any of Methods 1.122-1.127, wherein the method provides a significant reduction in the CGI-S score within 43 days of the beginning of treatment, e.g., at least a 1-point reduction of the CGI-S score, or at least a 1.5-point reduction, or at least a 1.6-point reduction, or about a 1.7-point reduction;
1.129. Any of Methods 1.122-1.128, wherein the method provides a significant reduction in the MADRS inner tension item score within 22 days of the beginning of treatment, e.g., at least a 0.5-point reduction of the MADRS inner tension item score, or at least a 0.6-point reduction, or at least a 0.7-point reduction, or about a 0.75-point reduction;
1.130. Any of Methods 1.122-1.129, wherein the method provides a significant reduction in the MADRS inner tension item score within 43 days of the beginning of treatment, e.g., at least a 0.9-point reduction of the MADRS inner tension item score, or at least a 1.0-point reduction, or at least a 1.1-point reduction, or about a 1.2-point reduction;
1.131. Any of Methods 1.122-1.130, wherein the method provides a significant reduction in the QIDS-SR-16 total score within 43 days of the beginning of treatment, e.g., at least a 6-point reduction of the QIDS-SR-16 total score, or at least a 7-point reduction, or at least an 8-point reduction, or about a 9-point reduction;
1.132. Any of Methods 1.122-1.131, wherein the method provides a significant reduction in the GAD-7 total score within 43 days of the beginning of treatment, e.g., at least a 3-point reduction of the GAD-7 total score, or at least a 4-point reduction, or at least a 5-point reduction, or about a 5.5-point reduction;
1.133. Any of Methods 1.122-1.132, wherein the administration of the lumateperone is adjunct to treatment with an antidepressant agent, e.g., an SSRI, SNRI, vortioxetine, bupropion, or mirtazapine;
1.134. Method 1.133, wherein the SSRI is selected from citalopram, escitalopram, fluoxetine, fluvoxamine, paroxetine, and sertraline;
1.135. Method 1.134, wherein the SNRI is selected from desvenlafaxine, duloxetine, levomilnacipran, milnacipran, tofenacin, and venlafaxine;
1.136. Method 1, or any of 1.1-1.135, wherein the method provides continued improvement in symptoms of depression and/or anxiety, e.g., as measured by one or more of MADRS Total score, CGI-S score, QIDS-SR-16 total score, or GAD-7 total score, for at least 6 weeks after the start of treatment, e.g., at least 8 weeks, at least 10 weeks, at least 12 weeks, at least 16 weeks, at least 20 weeks, or at least 24 weeks, at least 28 weeks, at least 32 weeks, or at least 36 weeks;
1.137. Method 1, or any of 1.1-1.136, wherein the method results in at least a 40% rate of response in treated patients, e.g., at least 50%, at least 60%, at least 70%, or about 80%, wherein response is defined as at least a 50% decrease from baseline in symptoms (e.g., as measured by one or more of MADRS Total score, CGI-S score, QIDS-SR-16 total score, or GAD-7 total score, e.g., at least 40% after 6 weeks of treatment and/or at least 70% after 6 months of treatment;
1.138. Method 1, or any of 1.1-1.137, wherein the method results in at least a 20% rate of remission in treated patients, e.g., at least 25%, at least 30%, at least 40%, at least 50%, at least 60%, or about 65%, wherein remission is defined as reduction in symptoms to normal levels (e.g., as measured by one or more of MADRS Total score, CGI-S score, QIDS-SR-16 total score, or GAD-7 total score), for example, an MADRS Total score of less than or equal to 10, e.g., at least 20% after 6 weeks of treatment and/or at least 60% after 6 months of treatment;
1.139. Method 1, or any of 1.1-1.138, wherein the method provides a significant reduction in the MADRS total score within 8 days of the beginning of treatment, e.g., at least a 2-point reduction on the MADRS total score, or at least a 3-point reduction, or about a 4-point reduction;
1.140. Method 1, or any of 1.1-1.139, wherein the method provides a significant reduction in the MADRS total score within 15 days of the beginning of treatment, e.g., at least a 6-point reduction on the MADRS total score, or at least a 7-point reduction, or about an 8-point reduction;
1.141. Method 1, or any of 1.1-1.140, wherein the method provides a significant reduction in the MADRS total score within 22 days of the beginning of treatment, e.g., at least an 8-point reduction on the MADRS total score, or at least a 9-point reduction, or about a 10-point reduction;
1.142. Method 1, or any of 1.1-1.141, wherein the method provides a significant reduction in the MADRS total score within 43 days of the beginning of treatment, e.g., at least a 10-point reduction on the MADRS total score, or at least a 12-point reduction, or about a 15-point or 4.5-point reduction;
1.143. Method 1, or any of 1.1-1.142, wherein the method provides a significant reduction in the CGI-S score within 8 days of the beginning of treatment, e.g., at least a 0.1-point reduction on the CGI-S score, or at least a 0.2-point reduction, or about a 0.3-point reduction;
1.144. Method 1, or any of 1.1-1.143, wherein the method provides a significant reduction in the CGI-S score within 15 days of the beginning of treatment, e.g., at least a 0.5-point reduction on the CGI-S score, or at least a 0.6-point reduction, or about a 0.7-point reduction;
1.145. Method 1, or any of 1.1-1.144, wherein the method provides a significant reduction in the CGI-S score within 22 days of the beginning of treatment, e.g., at least a 0.7-point reduction on the CGI-S score, or at least a 0.85-point reduction, or about a 1-point reduction;
1.146. Method 1, or any of 1.1-1.145, wherein the method provides a significant reduction in the CGI-S score within 43 days of the beginning of treatment, e.g., at least a 1.1-point reduction on the CGI-S score, or at least a 1.3-point reduction, or about a 1.6-point reduction;
1.147. Method 1, or any of 1.1-1.146, wherein the method provides a significant reduction in the QIDS-SR-16 score within 43 days of the beginning of treatment, e.g., at least a 6-point reduction on the QIDS-SR-16 score, or at least a 7-point reduction, or about an 8-point reduction;
1.148. Method 1, or any of 1.1-1.147, wherein the patient has a Young Mania Rating Scale (YMRS) score of 0-19, e.g., 4-16 or 6-12, or 8-10, at the beginning of treatment;
1.149. Method 1, or any of 1.1-1.148, wherein the patient has, or is suffering from, a major depressive episode characterized by an MADRS Total score of at least 24 and a CGI-S score of at least 4 (e.g., MADRS Total Score of 24-60 or 25-50, or 26-55, or 27-50, or 28-45, or 29-40, or 30-35, and/or a CGI-S score of 4-7, or 4-6, or 4-5);
1.150. Method 1, or any of 1.1-1.149, wherein the MDD is characterized by at least one major depressive episode characterized by an MADRS Total score of at least 24 and a CGI-S score of at least 4; 1.151. Method 1, or any of 1.1-1.150, wherein the patient is diagnosed as having MDD with mixed features (e.g., according to DSM-5 or DSM-5-TR);
1.152. Method 1, or any of 1.1-1.150, wherein the patient is diagnosed as having MDD without mixed features;
1.153. Method 1, or any of 1.1-1.152, wherein the patient has or suffers from anhedonia, e.g., as defined by an MADRS anhedonia factor score of at least 7 (e.g., 7-25, or 7-20, or 7-15, or 15 to 20);
1.154. Method 1, or any of 1.1-1.153, wherein the patient has an MADRS anhedonia factor score of at least 7 (e.g., 7-25, or 7-20, or 7-15) at the beginning of treatment;
1.155. Method 1, or any of 1.1-1.153, wherein the patient has an MADRS anhedonia factor score of at least 15 (e.g., 15-25, or 15-20, or 18-25, or 18-20) at the beginning of treatment;
1.156. Any of Methods 1.153-1.155, wherein the patient has an item score of at least 2, or at least 3, or at least 4, or at least 5, on one or more of the MADRS anhedonia factor items (i.e., apparent sadness, reported sadness, concentration difficulties, lassitude, and inability to feel);
1.157. Any of Methods 1.153-1.156, wherein the patient has an item score of at least 2, or at least 3, or at least 4, on the MADRS apparent sadness item or on the MADRS reported sadness item, or both;
1.158. Method 1, or any of 1.1-1.157, wherein the method provides a significant reduction in the MADRS anhedonia factor score within 8 days of the beginning of treatment, e.g., at least a 0.5-point reduction on the MADRS anhedonia factor score, or at least a 1-point reduction, or at least a 1.5-point reduction, about a 2-point reduction;
1.159. Method 1, or any of 1.1-1.158, wherein the method provides a significant reduction in the MADRS anhedonia factor score within 15 days of the beginning of treatment, e.g., at least a 2-point reduction on the MADRS anhedonia factor score, or at least a 3-point reduction, or at least a 4-point reduction, or about a 4-point reduction, or about a 5-point reduction;
1.160. Method 1, or any of 1.1-1.159, wherein the method provides a significant reduction in the MADRS anhedonia factor score within 22 days of the beginning of treatment, e.g., at least a 4-point reduction on the MADRS anhedonia factor score, or at least a 5-point reduction, or about a 6-point reduction;
1.161. Method 1, or any of 1.1-1.160, wherein the method provides a significant reduction in the MADRS anhedonia factor score within 29 days of the beginning of treatment, e.g., at least a 6-point reduction on the MADRS anhedonia factor score, or at least a 7-point reduction, or about an 8-point reduction;
1.162. Method 1, or any of 1.1-1.161, wherein the method provides a significant reduction in the MADRS anhedonia factor score within 36 days of the beginning of treatment, e.g., at least a 7-point reduction on the MADRS anhedonia factor score, or at least an 8-point reduction, or about a 9-point reduction;
1.163. Method 1, or any of 1.1-1.162, wherein the method provides a significant reduction in the MADRS anhedonia factor score within 43 days of the beginning of treatment, e.g., at least a 7-point reduction on the MADRS anhedonia factor score, or at least an 8-point reduction, or at least a 9-point reduction, or about a 10-point reduction;
1.164. Method 1, or any of 1.1-1.163, wherein the method provides a significant reduction in one or more of the MADRS anhedonia factor items within 8 days, or within 15 days, or within 22 days, or within 29 days, or within 36 days, or within 43 days of the beginning of treatment;
1.165. Method 1.164, wherein the reduction in score in any one or more of the MADRS anhedonia items is at least 0.5-points, or at least 1.0 point, or at least 1.25-points, or at least 1.5-points, or at least 1.75-points, or at least 2-points;
1.166. Method 1.165, wherein the reduction in score is achieved within 43 days of the beginning of treatment;
1.167. Any of Methods 1.164-1.166, wherein the reduction in score is achieved in the apparent sadness item, the reported sadness item, the lassitude item, or the inability to feel item, or any combination thereof;
1.168. Any of Methods 1.164-1.166, wherein the reduction in score is achieved in the apparent sadness item, the reported sadness item, or the inability to feel item, or any combination thereof;
1.169. Any of Methods 1.164-1.166, wherein the reduction in score is achieved in the apparent sadness item or the reported sadness item, or both;
1.170. Any of Methods 1.164-1.166, wherein a reduction in score is achieved in the apparent sadness item within 8 days of the beginning of treatment;
1.171. Any of Methods 1.164-1.170, wherein a reduction in score is achieved in the apparent sadness item or in the reported sadness item, or both, within 15 days of the beginning of treatment;
1.172. Any of Methods 1.164-1.171, wherein a reduction in score is achieved in the lassitude item or the inability to feel item, or both, within 15 days of the beginning of treatment;
1.173. Any of Methods 1.164-1.172, wherein a reduction in score is achieved in the inability to feel item or the concentration difficulties item, or both, within 22 days of the beginning of treatment;
1.174. Method 1, or any of 1.1-1.173, wherein the patient has an MADRS anhedonia factor score of at least 18 at the beginning of treatment;
1.175. Method 1, or any of 1.1-1.174, wherein the method provides a significant reduction in the YMRS score from baseline within 8 days, or within 15 days, or within 22 days, or within 29 days, or within 36 days, or within 43 days of the beginning of treatment, e.g., within 43 days;
1.176. Method 1, or any of 1.1-1.175, wherein the MDD is characterized by, or wherein the patient suffers from, symptoms of sexual dysfunction (e.g., characterized by a CSFQ-14 score of 47 or less for a male patient, or 41 or less for a female patient);
1.177. Method 1.176, wherein the symptoms of sexual dysfunction are selected from decreased sexual desire, decreased sexual excitement, decreased sexual satisfaction (e.g., dyspareunia, priapism, genital numbness), and decreased orgasmic function (e.g., delayed ejaculation, anorgasmia, impotence);
1.178. Any preceding method, wherein the patient has been previously treated with an SSRI, such as citalopram, escitalopram, fluoxetine, fluvoxamine, paroxetine, or sertraline, and the treatment resulted in, or worsened, symptoms of sexual dysfunction;
1.179. Any preceding method, wherein the patient has been previously treated with an SNRI, such as desvenlafaxine, duloxetine, levomilnacipran, milnacipran, tofenacin, or venlafaxine, and the treatment resulted in, or worsened, symptoms of sexual dysfunction;
1.180. Any preceding method, wherein the patient has been previously treated with vortioxetine, bupropion, or mirtazapine, and the treatment resulted in, or worsened, symptoms of sexual dysfunction;
1.181. Any preceding method, wherein the patient has been previously treated with a tricyclic antidepressant (TCA), such as amitriptyline, amitriptyline oxide, clomipramine, desipramine, dibenzepin, dimetacrine, dosulepin, doxepin, imipramine, lofepramine, melitracen, melixeran, nitroxazepine, nortriptyline, noxiptiline, pipofezine, protriptyline, or trimipramine, and the treatment resulted in, or worsened, symptoms of sexual dysfunction;
1.182. Any preceding method, wherein the patient has been previously treated with an antidepressant agent in combination with an antipsychotic, such as haloperidol, aripiprazole, quetiapine, olanzapine, risperidone, lurasidone, paliperidone, iloperidone, ziprasidone, brexpiprazole, asenapine, clozapine, or zotepine, and the treatment resulted in, or worsened, symptoms of sexual dysfunction;
1.183. Any of methods 1.178-1.182, wherein the sexual dysfunction resulted in non-adherence with the previous treatment (e.g., SSRI, SNRI, bupropion, etc.) or termination of the previous treatment (e.g., in favor of a different antidepressant treatment);
1.184. Method 1, or any of 1.1-1.183, wherein the patient suffers from persistent sexual dysfunction, e.g., sexual dysfunction lasting more than 1-month after cessation of prior antidepressant treatment (e.g., SSRI, SNRI, bupropion, etc.), such as more than 2-months, or more than 3-months, or more than 6-months, or more than 9-months, or more than 12-months;
1.185. Method 1, or any of 1.1-1.184, wherein the method does not result in the patient developing sexual dysfunction;
1.186. Method 1, or any of 1.1-1.184, wherein the method does not result in the worsening of sexual dysfunction;
1.187. Any preceding method, wherein the patient has a CSFQ-14 total score of 49 or less for a male or female patient, or 47 or less for a male patient, or 41 or less for a female patient, at the beginning of treatment;
1.188. Method 1.187, wherein the patient has a CSFQ-14 total score of less than 40, or less than 38, or less than 36, or less than 35, at the beginning of treatment;
1.189. Method 1.187 or 1.188, wherein the patient is a male having a CSFQ-14 total score of 47 or less at the beginning of treatment, e.g., 45 or less, or 43 or less, or 41 or less, or 39 or less, or 38 or less;
1.190. Method 1.187 or 1.188, wherein the patient is a female having a CSFQ-14 total score of 41 or less at the beginning of treatment, e.g., 39 or less, or 35 or less, or 32 or less, or 29 or less;
1.191. Method 1, or any of 1.176-1.190, wherein the method provides a significant increase in the CSFQ-14 total score within 43 days of the beginning of treatment, e.g., at least a 1.5-point increase, or at least a 2-point increase, or at least a 2.5-point increase, or at least a 3-point increase, or at least a 4-point increase, or about a 5-point increase;
1.192. Method 1, or any of 1.176-1.190, wherein the patient is a male, and the method provides a significant increase in the CSFQ-14 total score within 43 days of the beginning of treatment, e.g., at least a 0.5-point increase, or at least a 1.5-point increase, or at least a 2-point increase, or about a 2.2-point increase;
1.193. Method 1, or any of 1.176-1.190, wherein the patient is a female, and the method provides a significant increase in the CSFQ-14 total score within 43 days of the beginning of treatment, e.g., at least a 2-point increase, or at least a 2.5-point increase, or at least a 3-point increase, or at least a 3.5-point increase, or about a 3.8-point increase;
1.194. Method 1, or any of 1.176-1.193, wherein the patient has a CSFQ-14 total score greater than 37 after 43 weeks of treatment, or greater than 38, or greater than 39, or greater than 40, or greater than 41, or greater than 43, or greater than 45, or greater than 50, after 43 weeks of treatment;
1.195. Method 1, or any of 1.176-1.193, wherein the patient is a male, and wherein the patient has a CSFQ-14 total score greater than 37 after 43 weeks of treatment, or greater than 38, or greater than 39, or greater than 40, or greater than 45, or greater than 50, after 43 weeks of treatment;
1.196. Method 1, or any of 1.176-1.193, wherein the patient is a female, and wherein the patient has a CSFQ-14 total score greater than 31 after 43 weeks of treatment, or greater than 32, or greater than 33, or greater than 34, or greater than 35, or greater than 40, after 43 weeks of treatment;
1.197. Method 1, or any of 1.176-1.196, wherein method provides an increase in CSFQ-14 domain score (subscale score) after 43 weeks of treatment of at least 0.5 points for one or more of the domains pleasure, desire/frequency, desire/interest, arousal/excitement, and orgasm/completion;
1.198. Method 1, or any of 1.176-1.196, wherein method provides an increase in CSFQ-14 domain score (subscale score) after 43 weeks of treatment of at least 1 point for one or more of the domains desire/interest, arousal/excitement, and orgasm/completion;
1.199. Method 1, or any of 1.176-1.198, wherein the patient has one or more of the following CSFQ-14 domain scores (subscale scores) at the beginning of treatment: (a) pleasure score less than 3 or less than 2.5, (b) desire/frequency score less than 6 or less than 5, (c) desire/interest score less than 8 or less than 7 or less than 6, (d) arousal/excitement score less than 9, or less than 8, or less than 7, and (e) orgasm/completion score less than 9 or less than 8, or less than 7.5;
1.200. Method 1, or any of 1.1-1.199, wherein the administration of the lumateperone is adjunct to treatment with an antidepressant agent, e.g., an SSRI, SNRI, vortioxetine, bupropion, or mirtazapine, and the dose of the antidepressant agent is less than the recommended monotherapy dose of the antidepressant agent for treating MDD, e.g., and the method does not result in the onset or worsening of symptoms of sexual dysfunction (as defined herein above);
1.201. Method 1.200, wherein the SSRI is selected from citalopram, escitalopram, fluoxetine, fluvoxamine, paroxetine, and sertraline;
1.202. Method 1.200, wherein the SNRI is selected from desvenlafaxine, duloxetine, levomilnacipran, milnacipran, tofenacin, and venlafaxine;
1.203. Method 1, or any of 1.1-1.199, wherein the administration of the lumateperone is adjunct to treatment with an antidepressant agent, e.g., an SSRI, SNRI, vortioxetine, bupropion, or mirtazapine, and the dose of the antidepressant agent is the same as the recommended monotherapy dose of the antidepressant agent for treating MDD, e.g., and the method does not result in the onset or worsening of symptoms of sexual dysfunction (as defined herein above):
1.204. Method 1.203, wherein the SSRI is selected from citalopram, escitalopram, fluoxetine, fluvoxamine, paroxetine, and sertraline;
1.205. Method 1.203, wherein the SNRI is selected from desvenlafaxine, duloxetine, levomilnacipran, milnacipran, tofenacin, and venlafaxine;
1.206. Method 1, or any of 1.1-1.205, wherein the administration of the lumateperone is adjunct to treatment with an antidepressant agent, e.g., an SSRI, SNRI, vortioxetine, bupropion, or mirtazapine, and wherein the method prevents the onset or worsening of symptoms of sexual dysfunction (as defined herein above) associated with the antidepressant agent;
1.207. Method 1, or any of 1.1-1.206, wherein the patient is a male;
1.208. Method 1, or any of 1.1-1.206, wherein the patient is a female;
1.209. Method 1, or any of 1.1-1.208, wherein the patient is age 40 years or less (e.g., age 18-40, or 20-40, or 30-40, or 20-30, or 18-25);
1.210. Method 1, or any of 1.1-1.208, wherein the patient is greater than age 40 (e.g., age 40-80, or 40-70, or 40-60, or 40-50, or 50-70, or 50-60, or 60-70);
1.211. Method 1, or any of 1.1-1.210, wherein the patient is White;
1.212. Method 1, or any of 1.1-1.210, wherein the patient is non-White;
1.213. Method 1, or any of 1.1-1.212, wherein the patient is Hispanic or Latino;
1.214. Method 1, or any of 1.1-1.212, wherein the patient is not Hispanic or Latino;
1.215. Method 1, or any of 1.1-1.214, wherein the patient has a baseline MADRS Total score of less than or equal to 32 (e.g., 24 to 32, or 26 to 32, or 30 to 32), at the start of treatment;
1.216. Method 1, or any of 1.1-1.214, wherein the patient has a baseline MADRS Total score greater than 32 (e.g., 32 to 60, or 34 to 60, or 38 to 60, or 40 to 60, or 45 to 60), at the start of treatment;
1.217. Method 1, or any of 1.1-1.216, wherein the patient has not responded to one previous antidepressant treatment (ADT) regimen (e.g., during the current major depressive episode);
1.218. Method 1, or any of 1.1-1.216, wherein the patient has not responded to two or more previous antidepressant treatment (ADT) regimens (e.g., during the current major depressive episode);
1.219. Method 1, or any of 1.1-1.218, wherein the patient experiences any treatment-emergent adverse events with an onset of less than 3 weeks after the start of treatment (e.g., during the first week);
1.220. Method 1, or any of 1.1-1.219, wherein the patient experiences any treatment-emergent adverse events with a duration of less than 3 weeks (e.g., one week, or two weeks, or three weeks, or less than one week);
1.221. Method 1.219 or 1.220, wherein the treatment-emergent adverse events are selected from dizziness, dry mouth, somnolence, fatigue, nausea, and/or diarrhea;
1.222. Method 1, or any of 1.1-1.221, wherein the patient achieves, or the method provides, MADRS Total Score remission within 6 months of the beginning of treatment (MADRS Total Score of 10 or less), e.g., within 5 months, or within 4 months, or within 3 months, or within 2 months, or within 1 month, of the beginning of treatment;
1.223. Method 1, or any of 1.1-1.222, wherein the patient achieves, or the method provides, MADRS Total Score complete remission within 6 months of the beginning of treatment (MADRS Total Score of 5 or less), e.g., within 5 months, or within 4 months, or within 3 months, or within 2 months, or within 1 month, of the beginning of treatment;
1.224. Method 1, or any of 1.1-1.223, wherein the patient achieves, or the method provides, MADRS Total Score remission within 6 weeks of the beginning of treatment (MADRS Total Score of 10 or less), e.g., within 5 weeks, or within 4 weeks, or within 3 weeks, or within 2 weeks, or within 1 week, of the beginning of treatment;
1.225. Method 1, or any of 1.1-1.224, wherein the patient achieves, or the method provides, MADRS Total Score complete remission within 6 weeks of the beginning of treatment (MADRS Total Score of 5 or less), e.g., within 5 weeks, or within 4 weeks, or within 3 weeks, or within 2 weeks, or within 1 week, of the beginning of treatment;
1.226. Method 1, or any of 1.1-1.225, wherein the patient sustains, or the method provides, MADRS Total Score remission for at least 2 weeks (e.g., 15 days) following achievement of MADRS Total Score remission, e.g., for at least 3 weeks (e.g., 22 days), or at least 4 weeks (e.g., 29 days), or at least 5 weeks (e.g., 36 days), or at least 6 weeks (e.g., 43 days), or at least 8 weeks, or at least 12 weeks, or at least 14 weeks, or at least 18 weeks, or at least 22 weeks, or at least 26 weeks, or at least 30 weeks, or at least 32 weeks;
1.227. Method 1, or any of 1.1-1.226, wherein the method for the treatment of major depressive disorder (MDD) is a method for achieving remission of major depressive disorder;
1.228. Method 1, or any of 1.1-1.226, wherein the method for the treatment of major depressive disorder (MDD) is a method for achieving complete remission of major depressive disorder;
1.229. Method 1, or any of 1.1-1.226, wherein the method for the treatment of major depressive disorder (MDD) is a method for achieving sustained remission of major depressive disorder, e.g., sustained for at least 3 weeks (e.g., 22 days), or at least 4 weeks (e.g., 29 days), or at least 5 weeks (e.g., 36 days), or at least 6 weeks (e.g., 43 days), or at least 8 weeks, or at least 12 weeks, or at least 14 weeks, or at least 18 weeks, or at least 22 weeks, or at least 26 weeks, or at least 30 weeks, or at least 32 weeks;
1.230. Any preceding method, wherein the patient's MDD is characterized by a Quick Inventory of Depressive Symptomology-Self Report-16 Item (QIDS-SR-16) score of greater than or equal to 14 (e.g., greater than 17);
1.231. Any preceding method, wherein the major depressive episode is characterized by a Quick Inventory of Depressive Symptomology-Self Report-16 Item (QIDS-SR-16) score of greater than or equal to 14 (e.g., greater than 17);
1.232. Any preceding method, wherein the MDD or the major depressive episode is characterized at baseline as having a QIDS-SR-16 sleep disturbance score (on any one or more of the sleep disturbance items) of greater than 2 (e.g., on average, greater than 2.5, greater than 3, or greater than 3.5; or a score of 3 or 4);
1.233. Any preceding method, wherein the MDD or the major depressive episode is characterized at baseline as having a QIDS-SR-16 sad mood score (feeling sad score) of greater than 2 (e.g., on average, greater than 2.5, greater than 3, or greater than 3.5; or a score of 3 or 4);
1.234. Method 1, or any of 1.1-1.233, wherein the patient achieves, or the method provides, a significant reduction in the QIDS-SR-16 Total score within 43 days of the beginning of treatment, e.g., at least a 2-point decrease, or at least a 3-point decrease, or at least a 4-point decrease, or at least a 5-point decrease, or at least a 6-point decrease, or at least a 6.5-point decrease, or at least a 7-point decrease, or about a 7.5-point decrease, or about an 8-point decrease, or within 36 days, or within 29 days, or within 22 days, or within 15 days, or within 8 days;
1.235. Method 1, or any of 1.1-1.234, wherein the patient achieves, or the method provides, a significant reduction in one or more of the following QIDS-SR-16 symptom domain scores: sleep disturbance, sad mood, appetite/weight change, concentration, self-criticism, suicidal ideation, interest, energy/fatigue, and psychomotor agitation/retardation, within 43 days of the beginning of treatment, or within 36 days, or within 29 days, or within 22 days, or within 15 days, or within 8 days;
1.236. Method 1.235, wherein the patient achieves, or the method provides, a significant reduction in one or more of the following QIDS-SR-16 symptom domain scores: sleep disturbance, sad mood, appetite/weight change, concentration, interest, energy/fatigue, and psychomotor agitation/retardation, within 43 days of the beginning of treatment, or within 36 days, or within 29 days, or within 22 days, or within 15 days, or within 8 days;
1.237. Method 1.235, wherein the patient achieves, or the method provides, a significant reduction in one or more of the following QIDS-SR-16 symptom domain scores: sleep disturbance, sad mood, and interest, within 43 days of the beginning of treatment, or within 36 days, or within 29 days, or within 22 days, or within 15 days, or within 8 days;
1.238. Method 1.235, wherein the patient achieves, or the method provides, a significant reduction in one or more of the following QIDS-SR-16 symptom domain scores: sleep disturbance, and sad mood, within 43 days of the beginning of treatment, or within 36 days, or within 29 days, or within 22 days, or within 15 days, or within 8 days;
1.239. Method 1.235, wherein the patient achieves, or the method provides, a significant reduction in the QIDS-SR-16 symptom domain score of sleep disturbance, within 43 days of the beginning of treatment;
1.240. Method 1.235, wherein the patient achieves, or the method provides, a significant reduction in the QIDS-SR-16 symptom domain score of sad mood, within 43 days of the beginning of treatment, or within 36 days, or within 29 days, or within 22 days, or within 15 days, or within 8 days;
1.241. Any one of Methods 1.235-1.240, wherein the significant reduction in the one or more QIDS-SR-16 symptom domain scores is, independently, at least a 0.5-point decrease, or at least a 1-point decrease, or at least a 1.5-point decrease, or at least a 2-point decrease, or at least a 2.5-point decrease, or a 3-point decrease, e.g., at least a 1-point decrease or at least a 2-point decrease
1.242. Any one of Methods 1.235-1.241, wherein the significant reduction in the one or more QIDS-SR-16 symptom domain scores is achieved within 8 days, or within 15 days, or within 22 days, or within 29 days, or within 36 days, or within less than 43 days, of the beginning of treatment;
1.243. Method 1, or any of 1.1-1.242, wherein the patient is an adult and is underweight at the beginning of treatment, e.g., having a body mass index (BMI) of less than 18.5 kg/m²;
1.244. Method 1, or any of 1.1-1.242, wherein the patient is an adult and has a normal weight at the beginning of treatment, e.g., having a body mass index (BMI) of greater than or equal to 18.5 kg/m², and less than 25 kg/m²;
1.245. Method 1, or any of 1.1-1.242, wherein the patient is an adult and is overweight at the beginning of treatment, e.g., having a body mass index (BMI) of greater than or equal to 25 kg/m², and less than 30 kg/m²;
1.246. Method 1, or any of 1.1-1.242, wherein the patient is an adult and is obese at the beginning of treatment, e.g., having a body mass index (BMI) of greater than or equal to 30 kg/m²;
1.247. Method 1, or any of 1.1-1.246, wherein the patient's BMI is unchanged at 43 days after the beginning of treatment (e.g., <0.25 point change in BMI);
1.248. Method 1, or any of 1.1-1.246, wherein the patient's BMI is increased by not more than 0.5 points at 43 days after the beginning of treatment (e.g., <0.25 point increase in BMI);
1.249. Method 1, or any of 1.1-1.246, wherein the patient's BMI is decreased at 43 days after the beginning of treatment (e.g., >0.25 point decrease in BMI, or >0.5 point decrease, or >1 point decrease, or >2 point decrease, or >3 point decrease, or >4 point decrease, or >5 point decrease);
1.250. Method 1, or any of 1.1-1.249, wherein the patient's waist circumference is unchanged at 43 days after the beginning of treatment (e.g., <0.3 cm change), or wherein the patient's waist circumference is reduced at 43 days after the beginning of treatment (e.g., >0.3 cm reduction, or >0.5 cm reduction, or >1 cm reduction, or >1.5 cm reduction, or >2 cm reduction).

In another aspect, the disclosure provides lumateperone (e.g., lumateperone tosylate) for use in the treatment of major depressive disorder, e.g., for use in any of Methods 1, et seq.

In another aspect, the disclosure provides the use of lumateperone (e.g., lumateperone tosylate), in the manufacture of a medicament for the treatment of major depressive disorder, e.g., for use in any of Methods 1, et seq.

Lumateperone has high binding affinity for serotonin 5-HT_{2A} receptors (Ki=0.54 nM) and moderate binding affinity for dopamine D₂ (Ki=32 nM) receptors. Lumateperone has moderate binding affinity for dopamine D₁ (Ki=41 nM) and D₄ and adrenergic alpha_{1A} and alpha_{1B} receptors (Ki<100 nM), serotonin transporters (Ki=33 nM) and inhibits uptake of serotonin in cells expressing the human SERT (IC₅₀=150 nM). Lumateperone has low binding affinity (less than 50% inhibition at 100 nM) for muscarinic and histaminergic receptors.

In healthy human volunteers, the mean cortical 5-HT_{2A} receptor occupancy and the mean striatal D₂ receptor occupancy were greater than 80% and approximately 12%, respectively, after a single dose of lumateperone 7 mg. D₂ receptor occupancy increased with dose from 7 mg to 28 mg. In patients with schizophrenia treated for 2 weeks with lumateperone 42 mg, mean dorsal striatal D₂ receptor occupancy was 39%.

As a such, lumateperone has a very unique receptor binding profile that is not matched by any other existing antipsychotic or antidepressant medications, whether typical or atypical antipsychotics or any of the various classes of antidepressants. Nevertheless, lumateperone is currently classified as a member of the atypical antipsychotic class, despite its approval for treating depressive episodes associated with bipolar depression.

The precise mechanism of action of lumateperone in the treatment of schizophrenia, bipolar depression, and major depressive disorder is unknown. However, the mechanism of action of lumateperone for these indications could be mediated through a combination of antagonist activity at central serotonin 5-HT_{2A} receptors, and partial agonist activity at central dopamine D₂ receptors (e.g., presynaptic partial agonist and postsynaptic antagonist activity at central dopamine D₂ receptors).

Without being bound by theory, it is believed that, while lumateperone demonstrates inhibition of SERT in vitro, in vivo at clinically therapeutic doses for the treatment of MDD, SERT inhibition may not play a critical role in the mechanism of action. It is believed that the therapeutic efficacy of lumateperone in treating MDD is driven largely, if not completely, by NMDA and AMPA mediated mechanisms. This may account for its unexpectedly rapid onset of action in providing therapeutic relief for MDD symptoms. This is in striking contrast to traditional antidepressant agents, especially the SSRI and SNRI class, which derive both their therapeutic efficacy and their significant adverse event profile, primarily from SERT inhibition. In addition, while lumateperone demonstrates presynaptic partial agonist and postsynaptic antagonist activity at central dopamine D₂ receptors in vitro and/or in animal studies, and is believed to do the same in patients treated with lumateperone, it has not yet been definitively established that both presynaptic partial agonist and postsynaptic antagonist activity of D2 receptors are clinically relevant at therapeutic doses.

### Precautions for Use

Elderly patients with dementia-related psychosis treated with antipsychotic drugs are at an increased risk of death. Analyses of 17 placebo-controlled trials (modal duration of 10 weeks), largely in patients taking atypical antipsychotic drugs, revealed a risk of death in the drug-treated patients of between 1.6 to 1.7 times that in placebo-treated patients. Over the course of a typical 10-week controlled trial, the rate of death in drug-treated patients was about 4.5%, compared to a rate of about 2.6% in placebo-treated patients.

Although the causes of death were varied, most of the deaths appeared to be either cardiovascular (e.g., heart failure, sudden death) or infectious (e.g., pneumonia) in nature.

In placebo-controlled trials in elderly subjects with dementia, patients randomized to risperidone, aripiprazole, and olanzapine had a higher incidence of stroke and transient ischemic attack, including fatal stroke, compared to those treated with placebo.

It is believed that lumateperone (e.g., lumateperone tosylate) does not present the same risks in elderly patients and patients with dementia-related psychosis that other atypical antipsychotics do. Nevertheless, lumateperone tosylate is not currently approved in the United States for the treatment of patients with dementia-related psychosis.

In pooled analyses of placebo-controlled trials of antidepressant drugs (SSRIs and other antidepressant classes) that included approximately 77,000 adult patients and 4,500 pediatric patients, the incidence of suicidal thoughts and behaviors in antidepressant-treated patients age 24 years and younger (pediatric and young adult) was greater than in placebo-treated patients. There was considerable variation in risk of suicidal thoughts and behaviors among drugs, but there was an increased risk identified in young patients for most drugs studied. There were differences in absolute risk of suicidal thoughts and behaviors across the different indications, with the highest incidence in patients with MDD. It is unknown whether the risk of suicidal thoughts and behaviors in children, adolescents, and young adults extends to longer-term use, i.e., beyond four months. However, there is substantial evidence from placebo-controlled maintenance trials in adults with MDD that antidepressants delay the recurrence of depression and that depression itself is a risk factor for suicidal thoughts and behaviors.

It is believed that lumateperone (e.g., lumateperone tosylate) does not present the same risks of suicidal thoughts in children, adolescents, and young adults as other antidepressant agents. Nevertheless, lumateperone tosylate is not currently approved in the United States for the treatment of children, adolescents, and young adults. Moreover, all antidepressant-treated patients should be monitored for any indication for clinical worsening and emergence of suicidal thoughts and behaviors, especially during the initial few months of drug therapy, and at times of dosage changes

Neuroleptic Malignant Syndrome (NMS), a potentially fatal symptom complex, has been reported in association with administration of antipsychotic drugs. Clinical manifestations of NMS are hyperpyrexia, muscle rigidity, delirium, and autonomic instability. Additional signs may include elevated creatinine phosphokinase, myoglobinuria (rhabdomyolysis), and acute renal failure. If NMS is suspected in a patient, then such antipsychotic drug should be immediately discontinued, intensive symptomatic treatment and monitoring provided.

It is believed that lumateperone (e.g., lumateperone tosylate) does not present the same risks of NMS as do other atypical antipsychotics.

Tardive dyskinesia, a syndrome consisting of potentially irreversible, involuntary, dyskinetic movements, may develop in patients treated with antipsychotic drugs. The risk appears to be highest among the elderly, especially elderly women, but it is not possible to predict which patients are likely to develop the syndrome. Whether antipsychotic drug products differ in their potential to cause tardive dyskinesia is unknown.

The risk of tardive dyskinesia and the likelihood that it will become irreversible increase with the duration of treatment and the cumulative dose. The syndrome can develop after a relatively brief treatment period, even at low doses. It may also occur after discontinuation of treatment.

Tardive dyskinesia may remit, partially or completely, if antipsychotic treatment is discontinued. Antipsychotic treatment itself, however, may suppress (or partially suppress) the signs and symptoms of the syndrome, possibly masking the underlying process. The effect that symptomatic suppression has upon the long-term course of tardive dyskinesia is unknown.

Antipsychotics are therefore generally prescribed in a manner most likely to reduce the risk of tardive dyskinesia. Chronic antipsychotic treatment should generally be reserved for patients: 1) who suffer from a chronic illness that is known to respond to antipsychotic drugs; and 2) for whom alternative, effective, but potentially less harmful treatments are not available or appropriate. In patients who do require chronic treatment, use the lowest dose and the shortest duration of treatment producing a satisfactory clinical response. The need for continued treatment should be periodically reassessed.

If signs and symptoms of tardive dyskinesia appear in a patient on antipsychotic drug treatment, drug discontinuation should be considered. However, some patients may require treatment with the antipsychotic drug despite the presence of the syndrome.

Antipsychotic drugs have caused metabolic changes, including hyperglycemia, diabetes mellitus, dyslipidemia, and weight gain. Although many of the drugs in the antipsychotic class have been shown to produce some metabolic changes, each drug has its own specific risk profile.

Hyperglycemia, in some cases extreme and associated with ketoacidosis, hyperosmolar coma or death, has been reported in patients treated with antipsychotics. There have been reports of hyperglycemia in patients treated with lumateperone tosylate. Fasting plasma glucose should be assessed before or soon after initiation of antipsychotic medication and monitored periodically during long-term treatment.

In pooled data from short-term (4- to 6-week), placebo-controlled trials of adult patients with schizophrenia, mean changes from baseline and the proportion of patients with shifts from normal to greater than normal levels of fasting glucose in patients treated with lumateperone tosylate were similar to those in patients treated with placebo.

In an uncontrolled open-label trial of lumateperone tosylate for up to 1 year in patients with stable schizophrenia, the percentages of patients with a shift from normal to high in fasting glucose and insulin values were 8% and 12%, respectively. A total of 4.7% of patients with normal hemoglobin A1c (<6.5%) at baseline developed elevated levels (≥6.5%) post-baseline.

In data from short-term (6-week), placebo-controlled monotherapy and adjunctive therapy bipolar depression trials, mean changes from baseline and the proportion of patients with shifts from normal to greater than normal levels of fasting glucose and insulin in patients treated with lumateperone tosylate were similar to those in patients treated with placebo.

In pooled data from short-term (6-week), placebo-controlled adjunctive therapy MDD trials, mean changes from baseline and the proportion of patients with shifts from normal to greater than normal levels of fasting glucose in patients treated with lumateperone tosylate were similar to those in patients treated with placebo.

Antipsychotics have caused adverse alterations in lipids. Before or soon after initiation of antipsychotic medications, patients should obtain a fasting lipid profile at baseline and this should be monitored periodically during treatment.

In pooled data from short-term (4- to 6-week), placebo-controlled trials of adult patients with schizophrenia, mean changes from baseline and the proportion of patients with shifts to higher levels of fasting total cholesterol and triglycerides were similar in patients treated with lumateperone tosylate and placebo.

In an uncontrolled open-label trial of lumateperone tosylate for up to 1 year in patients with stable schizophrenia, the percentages of patients with a shift from normal to high were 8%, 5%, and 4% for total cholesterol, triglycerides, and LDL cholesterol, respectively.

In data from short-term (6-week), placebo-controlled monotherapy and adjunctive therapy bipolar depression trials, mean changes from baseline and the proportion of patients with shifts to higher levels of fasting total cholesterol and triglycerides were similar in patients treated with lumateperone tosylate and placebo.

In an uncontrolled open-label trial of lumateperone tosylate for up to 6 months in patients with bipolar depression, the proportion of patients with a shift from normal to high were 10%, 5%, and 2% for total cholesterol, triglycerides, and LDL cholesterol, respectively.

In pooled data from short-term (6-week), placebo-controlled adjunctive therapy MDD trials, mean changes from baseline and the proportion of patients with shifts to higher levels of fasting total cholesterol and triglycerides were similar in patients treated with lumateperone tosylate and placebo.

Weight gain has been observed with use of antipsychotics, often severe weight gain. Patients should have their weight monitored at baseline and frequently thereafter. In pooled data from placebo-controlled trials of adult patients with schizophrenia, mean changes from baseline and the proportion of subjects with an increase in weight ≥7% from baseline to end of study was similar in patients treated with lumateperone tosylate and placebo.

In an uncontrolled open-label trial of lumateperone tosylate for up to 1 year in patients with stable schizophrenia, the mean change in body weight was approximately −2 kg (SD 5.6) at Day 175 and approximately −3.2 kg (SD 7.4) at Day 350.

In data from short-term (6-week), placebo-controlled monotherapy and adjunctive therapy bipolar depression trials, mean changes from baseline and the proportion of patients with an increase in weight ≥7% from baseline to end of study were similar in patients treated with lumateperone tosylate and placebo.

In an uncontrolled open-label trial of lumateperone tosylate for up to 6 months in patients with bipolar depression, the mean change in body weight was −0.01 kg (SD 3.1) at Day 175.

In pooled data from short-term (6-week), placebo-controlled adjunctive therapy MDD trials, mean changes from baseline and the proportion of patients with an increase in weight ≥7% from baseline to end of study were similar in patients treated with lumateperone tosylate and placebo.

In a long-term open-label adjunctive therapy MDD trial of lumateperone tosylate for up to 6 months, the mean change in body weight was 0.05 kg (SD 3.6) at Week 26.

Leukopenia and neutropenia have been reported during treatment with antipsychotic agents, including lumateperone tosylate. Agranulocytosis (including fatal cases) have been reported with other agents in the atypical antipsychotic class.

Possible risk factors for leukopenia and neutropenia include pre-existing low white blood cell count (WBC) or absolute neutrophil count (ANC) and history of drug-induced leukopenia or neutropenia. In patients with a pre-existing low WBC or ANC or a history of drug-induced leukopenia or neutropenia, a complete blood count (CBC) should be performed frequently during the first few months of therapy. In such patients, discontinuation of treatment should be considered at the first sign of a clinically significant decline in WBC in the absence of other causative factors.

Patients with clinically significant neutropenia should be monitored for fever or other symptoms or signs of infection and treat promptly if such symptoms or signs occur. Treatment should be discontinued in patients with absolute neutrophil count <1000/mm³ and WBC values should be followed until recovery.

Atypical antipsychotics cause orthostatic hypotension and syncope. Generally, the risk is greatest during initial dose administration. In clinical trials, the frequencies of orthostatic hypotension for lumateperone tosylate and placebo were 0.7% and 0%, respectively. The rate of syncope for lumateperone tosylate and placebo were 0.2% and 0.2%.

In data from short-term (6-week), placebo-controlled monotherapy and adjunctive therapy bipolar depression trials, the frequencies of orthostatic hypotension for lumateperone tosylate and placebo were both 0%. The rates of syncope for lumateperone tosylate and placebo were 0.3% and 0.5%, respectively in the monotherapy trials, and there were no reports for lumateperone tosylate or placebo in the adjunctive therapy trial.

In pooled data from short-term (6-week), placebo-controlled adjunctive therapy MDD trials, the frequencies of orthostatic hypotension for lumateperone tosylate and placebo were 6.6% and 6.2% respectively. The rates of syncope for lumateperone tosylate and placebo were 0.2% and 0%.

Orthostatic vital signs should be monitored in patients who are vulnerable to hypotension (e.g., elderly patients, patients with dehydration, hypovolemia, and concomitant treatment with antihypertensive medications), patients with known cardiovascular disease (history of myocardial infarction, ischemic heart disease, heart failure, or conduction abnormalities), and patients with cerebrovascular disease. Lumateperone tosylate has not been evaluated in patients with a recent history of myocardial infarction or unstable cardiovascular disease. Such patients were excluded from pre-marketing clinical trials.

Antipsychotics, including lumateperone (e.g., lumateperone tosylate), may cause somnolence, postural hypotension, and motor and sensory instability, which may lead to falls and, consequently, fractures and other injuries. For patients with conditions or taking concomitant medications, that could exacerbate these effects, fall risk assessments should be conducted when initiating lumateperone treatment and periodically during long-term treatment.

Like other antipsychotic drugs, lumateperone (e.g., lumateperone tosylate), may cause seizures. The risk is greatest in patients with a history of seizures or with conditions that lower the seizure threshold. Conditions that lower the seizure threshold may be more prevalent in older patients.

Lumateperone (e.g., lumateperone tosylate), like other antipsychotics, may cause somnolence and has the potential to impair judgment, thinking, and motor skills. In short-term (i.e., 4- to 6-week) placebo-controlled clinical trials of patients with schizophrenia, somnolence and sedation were reported in 24% of lumateperone tosylate-treated patients, compared to 10% of placebo-treated patients. In short term (6-week), placebo-controlled monotherapy and adjunctive therapy bipolar depression clinical trials, somnolence and sedation were reported in 13% of lumateperone tosylate-treated patients, compared to 3% of placebo-treated patients. In short term (6-week), placebo-controlled adjunctive therapy MDD trials, somnolence and sedation were reported in 12% of lumateperone tosylate-treated patients, compared to 2% of placebo-treated patients.

Patients taking lumateperone (e.g., lumateperone tosylate) should be cautioned about operating hazardous machinery, including motor vehicles, until they are reasonably certain that therapy with lumateperone tosylate does not affect them adversely.

Atypical antipsychotics may disrupt the body's ability to reduce core body temperature. Strenuous exercise, exposure to extreme heat, dehydration, and anticholinergic medications may contribute to an elevation in core body temperature; lumateperone tosylate should be used with caution in patients who may experience these conditions.

Esophageal dysmotility (dysphagia) and aspiration have been associated with antipsychotic drug use. Antipsychotic drugs, including lumateperone (e.g., lumateperone tosylate), should be used cautiously in patients at risk for aspiration.

Due to its unique pharmacological profile, lumateperone (e.g., lumateperone tosylate) does not cause many of the significant adverse events and risks associated with other drugs which alter serotonin receptor activity, such as the SSRI and SNRI class antidepressant agents.

Unlike other antidepressant agents, lumateperone (e.g., lumateperone tosylate), can safely be used concurrently or following treatment with monoamine oxidate inhibitor (MAOI) agents. Other antidepressant agents carry a warning that treatment should wait at least 14 days after discontinuation of MAOI therapy (including linezolid or intravenous methylene blue) and that MAOI therapy should wait for at least 14 days after stopping antidepressant therapy. In addition, when a patient taking an SSRI or SNRI, or other similar antidepressant agents, needs to be treated with an MAOI, treatment with the antidepressant agent must cease before the patient can begin taking the MAOI. Thus, there may be a period of up to two weeks where the patient is without antidepressant therapy, raising the risk of worsening of symptoms, suicidal ideation, and even attempted suicide. No such warnings are necessary for lumateperone treatment because lumateperone can safely be administered along with an MAOI.

Traditional antidepressant agents, such as SSRIs and SNRIs, can precipitate serotonin syndrome, a potentially life-threatening condition. This risk is increased with concomitant use of other serotonergic drugs (e.g., SNRIs, triptans, tricyclic antidepressants, opioids), lithium, tryptophan, buspirone, and St. John's Wort) and with drugs that impair metabolism of serotonin, i.e., MAOIs.

Serotonin syndrome symptoms may include mental status changes (e.g., confusion, agitation, hallucinations, delirium, and coma), autonomic instability (e.g., tachycardia, labile blood pressure, dizziness, diaphoresis, flushing, hyperthermia), neuromuscular symptoms (e.g., tremor, rigidity, myoclonus, hyperreflexia, incoordination), seizures, and/or gastrointestinal symptoms (e.g., nausea, vomiting, diarrhea). Without being bound by theory, it is believed that in vivo, lumateperone does not interfere with serotonin reuptake sufficiently to cause this adverse event risk.

Many SSRIs and SNRIs are contraindicated in patients taking, or within 14 days of stopping MAOIs, and SSRI and SNRI patients must be monitored for the emergence of serotonin syndrome. Unlike other antidepressant agents, lumateperone (e.g., lumateperone tosylate) can be safely used without the risk of causing or exacerbating serotonin syndrome, and lumateperone (e.g., lumateperone tosylate) can be safely combined with other serotonergic drugs, including SSRIs, SNRIs, triptans, and tricyclic antidepressants, and drugs which alter serotonin metabolism, such as MAOIs, provided that appropriate caution is taken when combining lumateperone with CYP 3A4 inducers and CYP 3A4 inhibitors, as discussed herein. Without being bound by theory, it is believed that in vivo, lumateperone does not interfere with serotonin reuptake sufficiently to cause this drug-drug interaction risk.

Hyponatremia may occur as a result of treatment with other antidepressant agents, such as SSRIs and SNRIs. Patients treated with such agents have been reported to have serum sodium lower than 110 mmol/L. Signs and symptoms of hyponatremia include headache, difficulty concentrating, memory impairment, confusion, weakness, and unsteadiness, which may lead to falls. Signs and symptoms associated with more severe and/or acute cases have included hallucination, syncope, seizure, coma, respiratory arrest, and death. In many cases, SSRI/SNRI-associated hyponatremia appears to be the result of the syndrome of inappropriate antidiuretic hormone secretion (SIADH) and is reversible with antidepressant agent discontinuation. Elderly patients may be at greater risk of developing SSRI/SNRI-associated hyponatremia, and patients taking diuretics or who are otherwise volume depleted may be at greater risk. Unlike traditional antidepressant agents, lumateperone (e.g., lumateperone tosylate) can safely be used without significant risk of developing hyponatremia.

Drugs that interfere with serotonin reuptake inhibition, such as SSRIs and SNRIs, increase the risk of bleeding events. Concomitant use of aspirin, nonsteroidal anti-inflammatory drugs (NSAIDs), other antiplatelet drugs, warfarin, and other anticoagulants may add to the risk. Case reports and epidemiological studies (case-control and cohort design) have demonstrated an association between use of drugs that interfere with serotonin reuptake and the occurrence of gastrointestinal bleeding. Based on data from the published observational studies, exposure to other antidepressant agents, such as SSRIs and SNRIs, particularly in the month before delivery, has been associated with a less than 2-fold increase in the risk of postpartum hemorrhage Bleeding events related to drugs that interfere with serotonin reuptake have ranged from ecchymoses, hematomas, epistaxis, and petechiae to life-threatening hemorrhages. Unlike traditional antidepressant agents, lumateperone (e.g., lumateperone tosylate) can safely be used without significant risk of bleeding events. Without being bound by theory, it is believed that in vivo, lumateperone does not interfere with serotonin reuptake sufficiently to cause this adverse event risk.

The pupillary dilation that occurs following use of many antidepressant drugs, including SSRIs and SNRIs, may trigger an angle closure attack in a patient with anatomically narrow angles who does not have a patent iridectomy. This increases the risk of developing glaucoma or worsening of glaucoma. Unlike traditional antidepressant agents, lumateperone (e.g., lumateperone tosylate) can safely be used without significant risk of developing or exacerbating angle closure glaucoma.

Use of other antidepressant agents, especially SSRIs and SNRIs, may cause symptoms of sexual dysfunction. In male patients, SRI use may result in ejaculatory delay or failure, decreased libido, and erectile dysfunction. In female patients, SRI use may result in decreased libido and delayed or absent orgasm. There have been reports of long-lasting sexual dysfunction where the symptoms have continued despite discontinuation of SSRIs/SNRI treatment, and this is a very significant side effect of current antidepressant treatments. Unlike traditional antidepressant agents, lumateperone (e.g., lumateperone tosylate) can safely be used without significant risk of developing or exacerbating sexual dysfunction.

Unlike many other antidepressant agents, including SSRIs and SNRIs, sudden discontinuation of lumateperone therapy has not been shown to be associated with a discontinuation syndrome. For example, abrupt discontinuation or dose reduction of the SNRI venlafaxine has been found to be associated with the appearance of new symptoms, the frequency of which increased with increased dose level and with longer duration of treatment. Reported symptoms include agitation, anorexia, anxiety, confusion, impaired coordination and balance, diarrhea, dizziness, dry mouth, dysphoric mood, fasciculation, fatigue, flu-like symptoms, headaches, hypomania, insomnia, nausea, nervousness, nightmares, sensory disturbances (including shock-like electrical sensations), somnolence, sweating, tremor, vertigo, and vomiting. There have been post-marketing reports of serious discontinuation symptoms which can be protracted and severe. Completed suicide, suicidal thoughts, aggression and violent behavior have been observed in patients during reduction in antidepressant dosage, including discontinuation. Other post-marketing reports describe visual changes (such as blurred vision or trouble focusing) and increased blood pressure after stopping or reducing the dose of venlafaxine.

With other SNRIs, and SSRIs, there have also been reports of adverse events occurring upon discontinuation of these drugs, particularly when abrupt, including the following: irritability, lethargy, emotional lability, tinnitus, and seizures. For these antidepressant agents, a gradual reduction in the dose, rather than abrupt cessation, is recommended. In contrast, for lumateperone treatment, current evidence indicates that, treatment can be discontinued suddenly with the adverse effects associated with other antidepressant agents.

Several common antidepressant agents have significant activity as inhibitors of the CYP 2D6 isoenzyme. As a result of this activity, these agents cannot be combined with pimozide or thioridazine, two agents with narrow therapeutic index which are substrates of CYP 2D6. Pimozide is an antipsychotic agent used for treating Tourette's syndrome, schizophrenia, delusional disorder, and other psychoses. Thioridazine is also an antipsychotic agent, primarily used for treating schizophrenia. Both drugs can cause cardiac irregularities, particularly, QTc interval prolongation, which can cause ventricular arrhythmias. Fluoxetine, paroxetine, sertraline, amitriptyline, impramine, and nortriptyline are all CYP 2D6 inhibitors, and so they are generally contraindicated in patients taking pimozide or thioridazine. In contrast, lumateperone does not have significant CYP 2D6 inhibitor activity at therapeutic concentrations, and it can be used with pimozide or thioridazine.

Concomitant use of lumateperone (e.g., lumateperone tosylate) with moderate or strong CYP 3A4 inhibitors increases lumateperone exposure, which may increase the risk of adverse reactions. A reduced dosage of 21 mg daily or 10.5 mg daily should be administered to patients receiving moderate CYP 3A4 inhibitors or strong CYP 3A4 inhibitors, respectively. Moderate CYP 3A4 inhibitors include amprenavir, ciprofloxacin, cyclosporine, diltiazem, erythromycin, fluconazole, fluvoxamine, and verapamil. Strong CYP 3A4 inhibitors include clarithromycin, grapefruit juice, itraconazole, voriconazole, nefazodone, ritonavir, and nelfinavir.

Concomitant use of lumateperone (e.g., lumateperone tosylate) with CYP3A4 inducers decreases the exposure of lumateperone. Such concomitant use should therefore be avoided. CYP 3A4 inducers include carbamazepine, phenytoin, rifampin, St. John's wort, bosentan, efavirenz, etravirine, modafinil, nafcillin, aprepitant, armodafinil, pioglitazone, and prednisone.

Concomitant use of lumateperone (e.g., lumateperone tosylate) with UGT inhibitors may increase the exposure of lumateperone and/or its metabolites. Such concomitant use should therefore be avoided. UGT inhibitors include valproic acid and probenecid.

Controlled clinical studies of lumateperone tosylate did not include any patients aged 65 or older to determine whether or not they respond differently from younger patients.

Antipsychotic drugs increase the risk of death in elderly patients with dementia-related psychosis. While lumateperone tosylate is not approved for the treatment of patients with dementia-related psychosis, it is believed that lumateperone tosylate does not present the same risks in this regard as other atypical antipsychotics.

Use of lumateperone (e.g., lumateperone tosylate) in patients with moderate (Child-Pugh class B) to severe hepatic impairment (Child-Pugh class C) generally resulted in higher drug exposure compared to patients with normal hepatic function. Therefore, a dosage reduction to 21 mg daily is recommended for patients with moderate to severe hepatic impairment. The recommended dosage for patients with mild hepatic impairment (Child-Pugh A) is the same as for patients with normal hepatic function.

The use of multiple serotonin reuptake inhibitors (including SSRIs and SNRIs) may increase the risk of SRI-associated adverse events (e.g., serotonin syndrome or hyponatremia). In clinical trials of lumateperone tosylate with adjunctive SSRI and SNRI agents, no clinically significant drug interactions were observed. Thus, it is believed that lumateperone (e.g., lumateperone tosylate) may be used safely in combination with serotonin reuptake inhibitors (including SSRIs and SNRIs). Nevertheless, increased monitoring of such patients for SRI-associated adverse reactions is recommended.

The words “treatment” and “treating” are to be understood accordingly as embracing prophylaxis and treatment or amelioration of symptoms of disease and/or treatment of the cause of the disease. In particular embodiments, the words “treatment” and “treating” refer to prophylaxis or amelioration of symptoms of the disease.

The term “adult” means a patient of at least 18 years of age.

With reference to a patient's prior treatment, the terms “did not adequately improve” or “the treatment was ineffective or not adequately effective” means that the patient did not respond to treatment, or that the treatment resulted in less than a 50% improvement in symptoms, for example, as evaluated using a clinical scale, such as MADRS, CGI-S, or QIDS-SR-16 (e.g., total scores from one or more of these scales), or that the patient's current episode (e.g., major depressive episode) remains ongoing despite treatment (e.g., as determined according to DSM-IV, DSM-IV-TR, DSM-5, or DSM-5-TR guidelines).

“Patient” as used herein means a human patient, unless otherwise indicated.

Pharmaceutically acceptable salts of lumateperone include pharmaceutically acceptable acid addition salts, for example, toluenesulfonic acid addition salts (tosylate salts). Tosylate salts of lumateperone include the monotosylate salt and the bis-tosylate salt.

The term “lumateperone” as used herein without further specificity is understood to embrace lumateperone free base and all pharmaceutically acceptable salts of lumateperone, including, but not limited to, lumateperone tosylate, lumateperone besylate, lumateperone hydrochloride, lumateperone oxalate, lumateperone aminosalicylate, lumateperone cyclamate, lumateperone nosylate, and lumateperone mesylate. In some embodiments, said lumateperone in free base or pharmaceutically acceptable salt form is in a crystalline form.

As used herein, unless specified otherwise, the term “lumateperone tosylate” refers to either lumateperone monotosylate, lumateperone bistosylate, or any other acid addition salt form of lumateperone comprising toluenesulfonic acid (including salts having a base to acid ratio of less than 1:1 and mixed salts comprising other acid anions). In some embodiments, any reference herein to “lumateperone tosylate” may refer to lumateperone monotosylate. Currently, only lumateperone monotosylate has been studied in human clinical trials and given regulatory approval for human administration. Thus, references to “lumateperone tosylate” with respect clinical trial results and regulatory approval herein is understood to refer to lumateperone monotosylate.

Lumateperone tosylate is sold as CAPLYTA®.

Where dosages or amounts of a salt are given by weight, e.g., milligrams per day or milligrams per unit dose, the dosage amount of the salt is given as the weight of the corresponding free base, unless otherwise indicated.

The term “concurrently” or “concomitantly” when referring to a therapeutic use refers to administration of two or more active ingredients to a patient as part of a regimen for the treatment of a disease or disorder, whether the two or more active agents are given at the same or different times or whether given by the same or different routes of administrations. Concurrent administration of the two or more active ingredients may be at different times on the same day, or on different dates or at different frequencies.

As used herein, references to patients “at risk of” having or developing any side effects or disorders may refer to such patients having any family history of said side effects or disorders, or may refer such patients having symptoms or clinical test results consistent with the risk of having or developing said side effects or disorders. The evaluation of such risks is within the skill of the generalist or specialist physician. For example, a patient at risk of having or developing diabetes may be evaluated based on fasting blood glucose level, fasting blood insulin level, glucose tolerance test, serum hemoglobin A1c, family history of diabetes, other risk factors for diabetes (such as body weight, cardiovascular health). It is understood that for several of these factors, test result may indicate either the presence of diabetes or a heightened risk for developing diabetes. Similarly, a patient having or at risk of having dyslipidemia may be evaluated based on fasting blood lipid levels (e.g., cholesterol; HDL, LDL and VLDL lipoproteins; lipoprotein ratios; triglycerides), past history of elevated blood lipid levels, and family history of dyslipidemia or heart disease (e.g., ischemic heart disease, coronary artery disease), or genetic variations associated with lipid disease. Similarly, a patient having or at risk of having hepatic impairment (or gradations in hepatic impairment) may be evaluated based on blood or hepatic biopsy levels of hepatic enzymes (e.g., ALT, AST, alkaline phosphatase) or evidence of current or past hepatitis (e.g., circulating antibodies to hepatitis A, B or C antigens), known risk factors for hepatic impairment (e.g., chronic alcoholism), or evidence of early-stage hepatic disease (e.g., fatty liver disease). Similarly, a patient having or at risk of having cognitive or motor impairment may be evaluated based on a patient's clinical examination (including memory tests, and diagnostic imaging such as CT and MRI, history of seizures), family history or diagnosis of diseases such as Alzheimer's disease and Parkinson's disease, and history of trauma (such as acute head trauma or evidence of traumatic brain injury).

References to “weight gain” herein may refer to changes in weight of a patient and/or to a comparison of a patient's body mass index (BMI) to normalized BMI ranges for said patient's height.

Methods of synthesizing lumateperone and related compounds are known in art, and include the methods disclosed in in U.S. Pat. Nos. 6,548,493; 7,238,690; 6,552,017; 6,713,471; 7,183,282; 7,081,455; 8,309,722; U.S. RE39680, and U.S. RE39679, and US 2017/183350, the contents of each of which are incorporated by reference in their entirety. Salts of the Compounds of the Invention may also be prepared as similarly described in U.S. Pat. Nos. 6,548,493; 7,238,690; 6,552,017; 6,713,471; 7,183,282; 8,648,077; U.S. RE39680; U.S. RE39679; the contents of each of which are incorporated by reference in their entirety.

All references cited herein are hereby incorporated by referenced in their entireties. In the event of a conflict in a definition in the present disclosure and that of a cited reference, the present disclosure controls.

The disclosure is further illustrated in the following examples, which are meant to be exemplary and not limiting.

### Example 1: Pharmacokinetic Studies

Following once daily oral administration of lumateperone tosylate, lumateperone steady state is reached in about 5 days. Increase in steady-state exposure is approximately dose-proportional in the range of 3.5 mg to 56 mg. A large inter-subject variability in lumateperone PK parameters was observed, with coefficients of variation for Cₘₐₓ (peak plasma concentration) and AUC (area under the concentration vs time curve) ranging from 68% to 97% at steady state.

Absorption: After oral dosing, the absolute bioavailability of lumateperone is about 4.4%. Cₘₐₓ of lumateperone is reached approximately 1-2 hours after lumateperone tosylate dosing.

Food Effect: Ingestion of a high-fat meal with lumateperone tosylate lowered lumateperone mean Cₘₐₓ by 33% and increased mean AUC by 9%. Median Tₘₐₓ was delayed about 1 hour (from 1 hour at fasted state to 2 hours in the presence of food).

Distribution: Protein binding of lumateperone is 97.4% at 5 μM (about 70-fold higher than therapeutic concentrations) in human plasma. The volume of distribution of lumateperone following intravenous administration is about 4.1 L/kg.

Elimination: The clearance of lumateperone is approximately 27.9 L/hour and the terminal half-life is about 18 hours after intravenous administration.

Metabolism: Lumateperone is extensively metabolized with more than twenty metabolites identified in vivo. After a single ¹⁴C-labeled oral dose, lumateperone and glucuronidated metabolites represent about 2.8% and 51% of the total plasma radioactivity, respectively. In vitro studies show that multiple enzymes, including but not limited to, uridine 5′-diphospho-glucuronosyltransferases (UDP-glucuronosyltransferase, UGT) 1A1, 1A4, and 2B15, aldoketoreductase (AKR) 1C1, 1B10, and 1C4, and cytochrome P450 (CYP) 3A4, 2C8, and 1A2, are involved in the metabolism of lumateperone.

Excretion: In a human mass balance study, 58% and 29% of the radioactive dose was recovered in the urine and feces, respectively. Less than 1% of the dose was excreted as unchanged lumateperone in the urine.

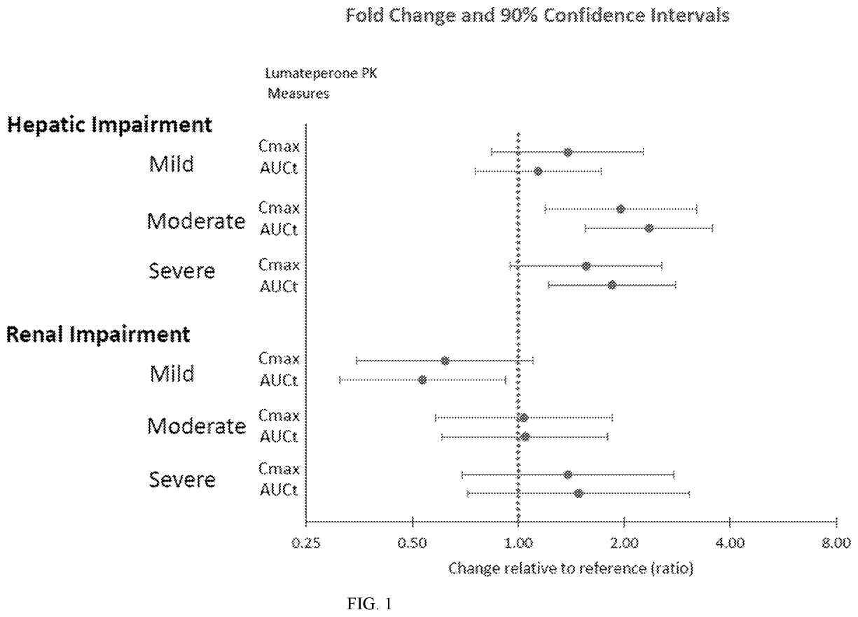

Specific Populations: Effects of hepatic or renal impairment on lumateperone exposure are presented in FIG. 1. No clinically significant differences in the pharmacokinetics of lumateperone were observed based on age, sex, or race.

Patients with Hepatic Impairment: The pharmacokinetics of lumateperone were evaluated in patients with hepatic impairment and in those with normal hepatic function following administration of a single 14 mg dose of lumateperone (20 mg lumateperone tosylate). In patients with mild hepatic impairment (Child-Pugh Class A) AUCt increased by 1.1-fold (0.8, 1.7) and Cₘₐₓ increased by 1.4-fold (0.8, 2.3) compared to subjects with normal hepatic function. In patients with moderate hepatic impairment (Child-Pugh Class B) AUCt increased by 2.4-fold (1.6, 3.6) and Cₘₐₓ increased by 2.0-fold (1.2, 3.2). In patients with severe hepatic impairment (Child-Pugh Class C) AUCt increased by 1.8-fold (1.2, 2.8) and Cₘₐₓ increased by 1.6-fold (0.9, 2.6).

Patients with Renal Impairment: The pharmacokinetics of lumateperone were evaluated in patients with renal impairment and in those with normal renal function following administration of a single 14 mg dose. In patients with mild renal impairment (eGFR of 60-89 mL/min/1.73 m²), geometric mean (90% confidence interval) AUCt decreased to 0.54 (0.31, 0.92), and geometric mean (90% confidence interval) Cₘₐₓ decreased to 0.62 (0.35, 1.10), compared to patients with normal renal function (eGFR>90 mL/min/1.73 m²). In patients with moderate renal impairment (eGFR of 30-59 mL/min/1.73 m²), geometric mean (90% confidence interval) AUCt increased by 1.0-fold (0.6, 1.8) and geometric mean (90% confidence interval) Cₘₐₓ increased by 1.0-fold (0.6, 1.9). In patients with severe renal impairment (eGFR<29 mL/min/1.73 m²) geometric mean (90% confidence interval) AUCt increased by 1.5-fold (0.8, 2.6) and Cₘₐₓ increased by 1.4-fold (0.8, 2.4).

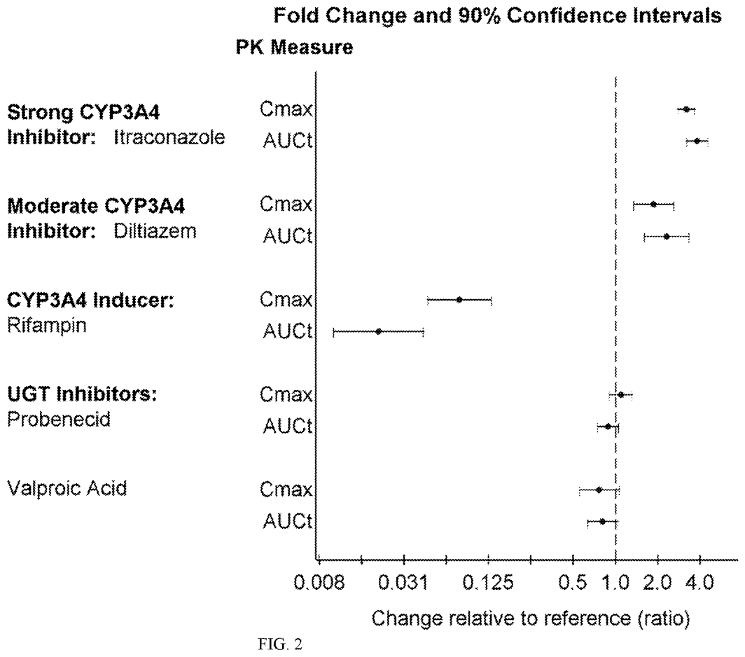

Drug Interaction Studies: The effects of other drugs on the exposures of lumateperone are presented in FIG. 2. Strong CYP3A4 Inhibitors: Itraconazole (strong CYP3A4 inhibitor) increased geometric mean (90% confidence interval) lumateperone AUCt by 3.8-fold (3.2, 4.5) and Cₘₐₓ by 3.2-fold (2.8-3.7). Moderate CYP3A4 Inhibitors: Diltiazem (moderate CYP3A4 inhibitor) increased geometric mean (90% confidence interval) lumateperone AUCt by 2.3-fold (1.6, 3.3) and Cₘₐₓ by 1.9-fold (1.3, 2.6). Strong CYP3A4 Inducers: Rifampin (strong CYP3A4 inducer) decreased geometric mean (90% confidence interval) lumateperone AUCt by 97.9% and Cₘₐₓ by 92.3%. UGT inhibitors: There were no clinically significant drug interactions with the UGT inhibitors probenecid and valproic acid. CYP3A4 substrates: No clinically significant differences in the pharmacokinetics of midazolam (CYP3A4 substrate) or its metabolite 1-hydroxymidazolam were observed when used concomitantly with single or multiple doses of lumateperone in patients with schizophrenia.

In Vitro Studies: Lumateperone showed little to no inhibition of CYP1A2, CYP2C9, CYP2C19, CYP2D6, or CYP3A4/5. It showed no induction of CYP1A2, CYP2B6, or CYP3A4. Lumateperone did not appear to be a P-gp or BCRP substrate. It showed little to no inhibition of OCT2, OAT1, OAT3, OATP1B3, or OATP1B1.

### Example 2: Clinical Trials Experience

### Adverse Events

Because clinical trials are conducted under widely varying conditions, adverse reaction rates observed in the clinical trials of a drug cannot be directly compared to rates in the clinical trials of another drug and may not reflect the rates observed in practice.

The safety of lumateperone tosylate (lumateperone monotosylate specifically) has been evaluated in at least 3575 adult patients with schizophrenia, bipolar depression, and major depressive disorder, exposed to one or more doses in clinical trials. A total of 852 lumateperone tosylate-treated patients had at least 6 months of treatment and 108 had at least 1 year of treatment with the 60-mg once daily dose of lumateperone tosylate (42 mg lumateperone free base).

Of these patients, 483 participated in short-term (6-week), placebo-controlled MDD adjunctive therapy studies in adult patients treated once daily with 60 mg of lumateperone tosylate. There was no single adverse reaction leading to discontinuation that occurred at a rate of >2% in lumateperone tosylate-treated patients.

The most common adverse reactions (incidence of at least 5% of patients treated with lumateperone tosylate and greater than twice the rate of placebo) were dizziness, dry mouth, somnolence/sedation, nausea, and fatigue.

Adverse reactions associated with lumateperone tosylate (incidence of at least 2% in patients treated with lumateperone tosylate and greater than placebo) are shown in the table below.

| Adverse Reactions Reported in ≥2% of | | Lumateperone |
|---|---|---|
| Lumateperone Tosylate-Treated Patients | Placebo | tosylate (60 mg) |
| in 6-Week Adjunctive MDD Trials | (N = 481) | (N = 483) |
| Headache (headache, migraine, tension | 13% | 19% |
| headache) | | |
| Dizziness (dizziness, postural dizziness) | 5% | 17% |
| Dry Mouth | 3% | 13% |
| Somnolence/Sedation | 2% | 12% |
| Nausea | 4% | 9% |
| Fatigue | 2% | 8% |
| Diarrhea | 1% | 5% |
| Tremor | <1% | 4% |
| Vomiting | 2% | 3% |
| Vertigo | <1% | 3% |
| Insomnia | 2% | 3% |

Dystonia: Symptoms of dystonia, prolonged abnormal contractions of muscle groups, may occur in susceptible individuals during the first few days of treatment. Dystonic symptoms include: spasm of the neck muscles, sometimes progressing to tightness of the throat, swallowing difficulty, difficulty breathing, and/or protrusion of the tongue. Although these symptoms can occur at low doses, they occur more frequently and with greater severity with high potency and higher doses of first-generation antipsychotic drugs. An elevated risk of acute dystonia is observed in males and younger age groups.

Extrapyramidal Symptoms (EPS): In the 6-week, adjunctive MDD trials, the frequency of reported EPS-related adverse reactions (tremor, bradykinesia, muscle spasms, gait disturbance, tongue spasm, muscle tightness and dyskinesia), excluding akathisia and restlessness, was 5% for lumateperone tosylate and 0.8% for placebo. The combined incidence of akathisia and restlessness was 1% for lumateperone tosylate and 0.8% for placebo. In the 6-week, adjunctive MDD trials, data were collected using the Simpson Angus Scale (SAS) for EPS (total score ranges from 0 to 40), the Barnes Akathisia Rating Scale (BARS) for akathisia (total score ranges from 0 to 14), and the Abnormal Involuntary Movement Scale (AIMS) for dyskinesia (total score ranges from 0 to 28). The mean changes from baseline for lumateperone tosylate-treated patients and placebo-treated patients were 0 and 0 for the SAS, 0 and −0.1 for the BARS, and 0 and 0 for the AIMS, respectively.

Cardiac Electrophysiology: QTcF interval was evaluated in a randomized, placebo- and active-(moxifloxacin 400 mg) controlled, four-arm crossover study utilizing concentration-QTc effect modeling in 33 patients with schizophrenia. The placebo-corrected change from baseline QTcF (90% two-sided upper confidence interval) values of 4.9 (8.9) and 15.8 (19.8) ms for the 42 mg and the supratherapeutic dose of 126 mg (three times the recommended daily dosage) lumateperone tosylate, respectively, administered orally once daily for 5 days.

### Study 5 and Study 6: Major Depressive Disorder

The effectiveness of lumateperone tosylate as adjunctive therapy with antidepressants (lumateperone tosylate 60 mg+ADT) for the treatment of major depressive disorder in adults was established in two 6-week, randomized, double-blind, placebo-controlled, multi-center trials in adult patients who met DSM-5 criteria for MDD, with or without anxiety (e.g., anxious distress) who had inadequate response to one to two courses of prior antidepressant (ADT) therapy (Study 5, NCT04985942, and Study 6, NCT05061706). Inadequate response during antidepressant treatment was defined as less than 50% improvement after six weeks of antidepressant treatment of adequate dose and adequate duration (e.g., SSRI, SNRI, or bupropion at minimum effective dosage). Patients were randomized to receive oral lumateperone tosylate (60 mg) or placebo once daily and continued their background ADT.

In Study 5 and 6, patients were enrolled who were experiencing a major depressive episode (defined as a MADRS total score greater than or equal to 24 and a CGI-S score of greater than or equal to 4), and who also had a QIDS-SR-16 score of greater than or equal to 14, both at initial screening and at baseline visit.

Endpoints: In Study 5 and 6, the primary endpoint was change from baseline to Week 6 (43 days) in the MADRS total score in the lumateperone+ADT group compared to the placebo+ADT group. Additional measures included response rate (≥50% MADRS Total score decrease) and remission rate (MADRS Total score <10), based on a logistic regression model.

The MADRS is a 10-item clinician-rated scale with total scores ranging from 0 (no depressive features) to 60 (maximum score). Thus, higher MADRS total scores reflect greater symptom severity.

The key secondary endpoint was the change from baseline to Week 6 (43 days) in the Clinical Global Impression Scale-Severity (CGI-S) score. The CGI-S is a validated clinician-rated scale that measures the patient's current illness state on a 1 (normal, not at all ill) to 7-point (extremely ill) scale. Patients had an MADRS total score of at least 24 and a CGI-S score of at least 4 at baseline for both Studies.

### Baseline Demographics

In Study 5, a total of 485 patients were randomized to receive lumateperone tosylate 60 mg (equivalent to 42 mg lumateperone free base) plus ADT or placebo plus ADT. Demographic and baseline characteristics were similar for the lumateperone tosylate and placebo groups. Median age was 46 (range 18 to 65). 66% were female, 77% were White, 15% were Asian, and 7% were Black. Mean MADRS Total Score at baseline was 30.4 in the lumateperone group and 30.1 in the placebo group, while mean CGI-S score at baseline was 4.7 in the lumateperone group and 4.6 in the placebo group.

In Study 6, a total of 480 patients were randomized to receive lumateperone tosylate 60 mg or placebo, each in combination with antidepressant therapy. Demographic and baseline characteristics were similar for the lumateperone tosylate and placebo groups. Median age was 48 (range 18 to 65). 70% were female, 95% were White. Mean MADRS Total Score at baseline was 30.8 in the lumateperone group and 31.5 in the placebo group, while mean CGI-S score at baseline was 4.6 in the lumateperone group and 4.7 in the placebo group.

### Efficacy Results

In Studies 5 and 6, patients randomized to lumateperone tosylate 60 mg+ADT showed a statistically significant improvement from baseline to day 43 in the MADRS total score and CGI-S score compared to the placebo+ADT group. The results of Study 5 and Study 6 for MADRS total score are shown in the following table:

| | | | Primary Efficacy Endpoint: MADRS Total Score | | |
|---|---|---|---|---|---|
| | | | Mean | LS Mean | Placebo − subtracted |
| | | | Baseline | Change from | Difference ^{#} |
| Study | Treatment Group | N | Score (SD) | Baseline (SE) | (95% CI) |
| 5 | Lumateperone tosylate | 239 | 30.4 (3.75) | −14.7 (0.54) | −4.9 (−6.38, −3.44) |
| | 60 mg + ADT* | | | | |
| | Placebo + ADT | 242 | 30.1 (3.50) | −9.8 (0.53) | |
| 6 | Lumateperone tosylate | 232 | 30.8 (3.88) | −14.7 (0.56) | −4.5 (−6.03, −3.02) |
| | 60 mg + ADT* | | | | |
| | Placebo + ADT | 237 | 31.5 (3.97) | −10.2 (0.54) | |

SD: standard deviation;
SE: standard error;
LS Mean: least squares mean;
CI: confidence interval;
*Statistically significantly superior to placebo;
^{#} Difference (drug minus placebo) in LS mean change from baseline.

The difference between the lumateperone group and the placebo group in change from baseline MADRS total score was substantial starting at Week 1 (Day 8).

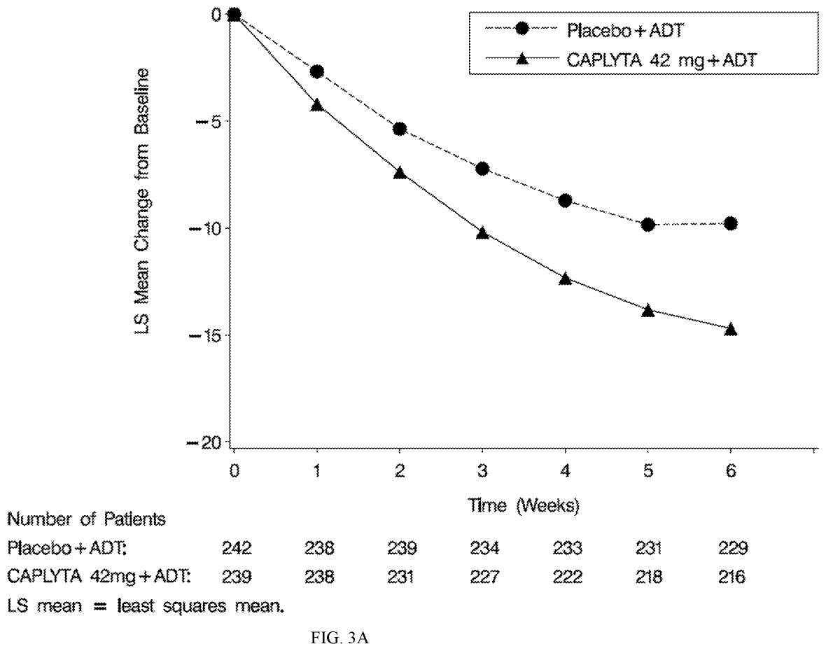

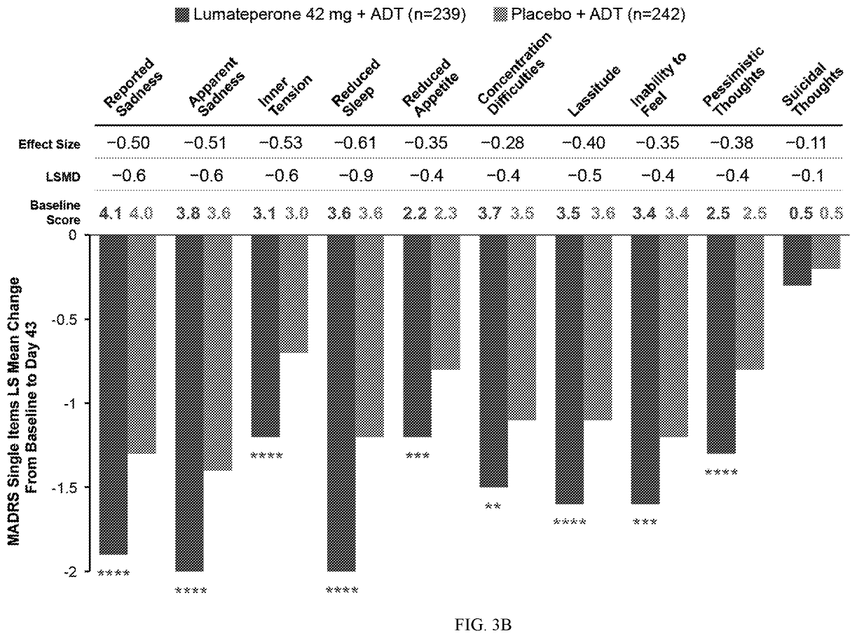

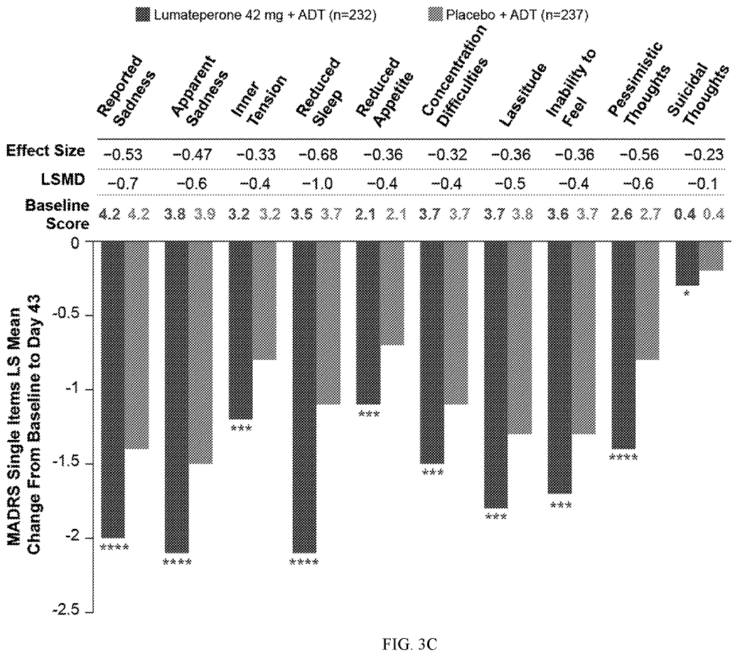

The change from baseline in MADRS total score by week in adult patients who received adjunctive treatment for MDD in Study 5 is displayed in FIG. 3A.

### Study 5 and 6: MADRS Single Item Scores

A prospective analysis investigated the change from baseline to Day 43 in each of the MADRS single item scores. At baseline in both Studies, the most prominent MADRS single items were reported sadness and apparent sadness (both elements of the MADRS anhedonia factor score).

In both studies, at Day 43, the lumateperone+ADT group showed significant improvements in all MADRS single item scores compared to placebo+ADT, except that for Study 5, the change in suicidal thoughts item score was not significant (suicidal thought items were very low at baseline in both studies.). The largest improvement occurred in the reported sadness, apparent sadness, and reduced sleep MADRS items in both studies. The results are shown in FIG. 3B and FIG. 3C.

In Study 5, the earliest significant reductions (P<0.05) in MADRS single item scores from baseline occurred at Day 8 for the reported sadness, apparent sadness, and reduced sleep item scores. Significant improvements for the inability to feel and pessimistic thoughts item scores were observed at Day 15. Significant improvements for the inner tension and concentration difficulties item scores were observed at Day 22, and for the reduced appetite and lassitude items scores by Day 29.

In Study 6, the earliest significant reductions (P<0.05) in MADRS single item scores from baseline occurred at Day 8 for the reduced sleep item score. Significant improvements for the reported sadness and apparent sadness item scores were observed by Day 15. Significant improvements for the inability to feel and pessimistic thoughts item scores were observed at Day 15. Significant improvements for the lassitude, inability to feel, and pessimistic thoughts item scores were observed at Day 22, and for the inner tension and concentration difficulties item scores by Day 29. A significant improvement in the reduced appetite item score was observed by Day 36, and in the suicidal thoughts item score only at Day 43.

Analysis of Pooled Efficacy Results from Study 5 and Study 6

Together, Study 5 and Study 6 included 471 patients treated with lumateperone+ADT and 479 patients treated with placebo+ADT in the modified intent to treat population (mITT) (483 lumateperone patients and 481 placebo patients in the intent-to-treat population (ITT)). The patients' baseline parameters are shown in the following table (MADRS and CGI-S are for mITT population; QIDS-SR-16 is for ITT population):

| | Lumateperone + | Placebo + |
|---|---|---|
| Baseline Efficacy Parameters | ADT | ADT |
| MADRS Total score, mean (SD) | 30.6 (3.82) | 30.8 (3.80) |
| CGI-S score, mean (SD) | 4.7 (0.57) | 4.6 (0.57) |
| QIDS-SR-16 Total score, mean (SD) | 18.0 (2.42) | 17.8 (2.40) |

Baseline ADT treatment was as follows:

| | Lumateperone + | Placebo + |
|---|---|---|
| Baseline ADT (safety | ADT | ADT |
| population) | (n = 483) | (n = 481) |
| SSRI (selective serotonin | 328 (68%) | 312 (65%) |
| reuptake inhibitor) | | |
| SNRI (serotonin-norepinephrine | 126 (26%) | 138 (29%) |
| reuptake inhibitor) | | |
| Bupropion | 29 (6%) | 31 (6%) |

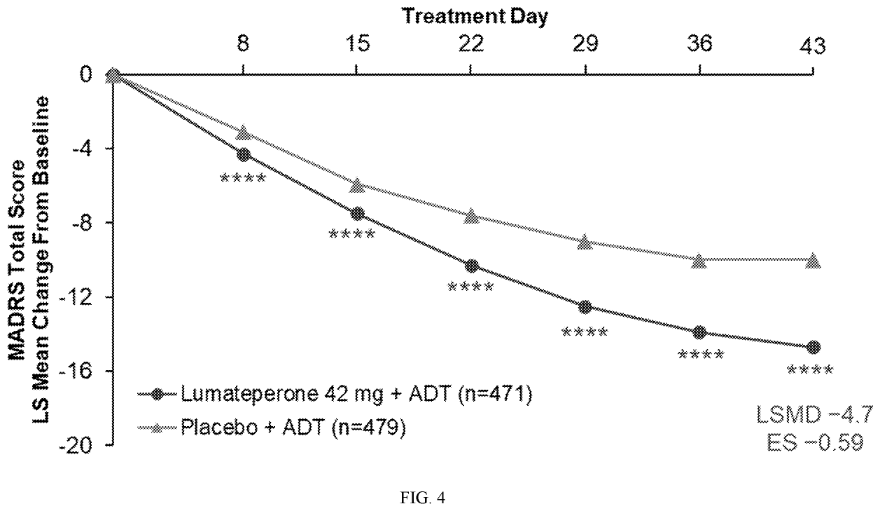

The pooled results show that the treatment group (lumateperone 42 mg+ADT) experienced significantly improved MADRS Total score at Day 43 compared with the placebo group (placebo+ADT), as shown in FIG. 4. Significantly greater MADRS Total score reductions were observed at the earliest assessment (Day 8) in the treatment group and persisted throughout the study.

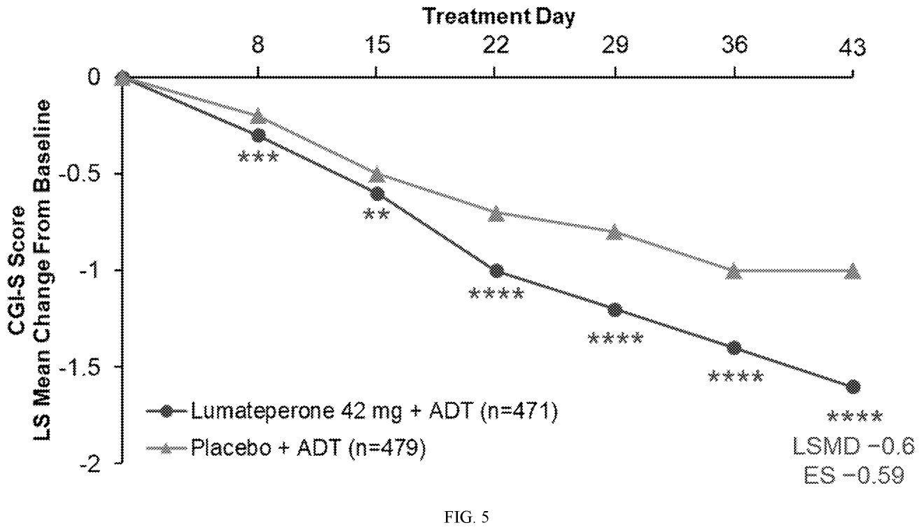

The pooled results also show that the treatment group was associated with improvements in overall MDD disease severity, as shown in FIG. 5. CGI-S score in the treatment group also showed improvements at the earliest assessment (Day 8), and this result persisted throughout the study.

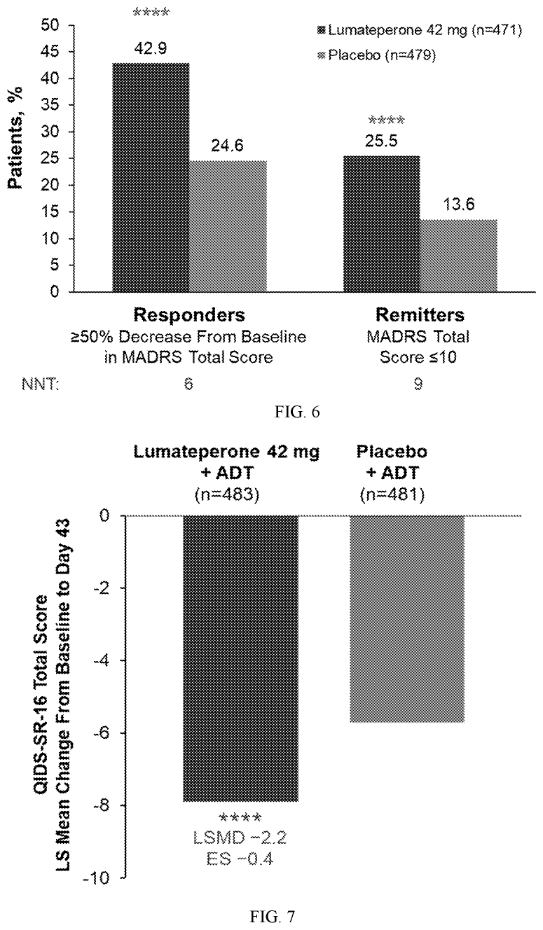

In the pooled population at Day 43, the rates of MADRS Total score response and remission were significantly greater in the treatment group compared to the placebo group, as shown in FIG. 6. Based on the number needed to treat (NNT), the treatment method was associated with clinically meaningful outcomes.

In addition, self-reported depressive symptoms, as measured by QIDS-SR-16 total score, was significantly improved in the treatment group compared with the placebo group, from baseline through Day 43, as shown in FIG. 7.

These results further demonstrate the unexpected efficacy of 42 mg lumateperone (60 mg lumateperone tosylate) in the treatment of major depressive disorder in patients receiving adjunctive antidepressant therapy.

In addition, the pooled results demonstrate that treatment-emergent adverse events (TEAEs) occurred in 68% of lumateperone patients (51% considered drug-related TEAEs), compared to 45% of placebo patients (20% drug-related). Serious TEAEs occurring in just one patient in each group (0.2%). Only 9% of lumateperone patients discontinued treatment due to adverse events (1% in placebo group). The TEAEs reported in at least 5% of lumateperone patients in at least double the placebo rate were minor: dizziness (16%), dry mouth (13%), somnolence (10%), nausea (9%), and fatigue (7%). 95% of TEAEs in the lumateperone group were mild to moderate in severity (98% in placebo group). Mean changes from baseline to end of treatment were minimal for body morphology (weight, BMI, waist circumference), cardiometabolic laboratory values (cholesterol, triglycerides, glucose, insulin), and prolactin levels, and these were comparable between lumateperone and placebo groups. No notable changes occurred in EPS in either group. Rates of emergence of suicidal ideation or suicidal behavior were low and similar between the groups (no suicidal behavior; suicidal ideation emergence, 1.6% in lumateperone group, 2.5% in placebo group (Columbia-Suicide Severity Rating Scale)).

### Study 5: QIDS-SR-16 Post-Hoc Analysis

As noted above, Study 5 involved a total of 485 patients randomized to receive lumateperone tosylate 60 mg (equivalent to 42 mg lumateperone free base) plus ADT or placebo plus ADT. The intent-to-treat (ITT) population included 484 patients, 241 in the lumateperone+ADT group, and 234 in the placebo+ADT group, and 452 of these patients completed treatment. The primary and secondary endpoints in the study were change from baseline to Day 43 in MADRS Total Score and CGI-S score, respectively. An additional measure used was the QIDS-SR-16 score and sub-scores.

The Quick Inventory of Depressive Symptomatology-Self Report (QIDS-SR-16) is a 16-item, validated self-report questionnaire used to assess the severity of depressive symptoms. The score is based on nine DSM-IV symptom domains: mood, concentration, self-outlook, suicidal ideation, energy, sleep disturbance (four items), appetite/weight changes (four items), and psychomotor agitation/retardation. Total scores range from 0 to 27. Commonly, the following labels are applied to the total scores: 0-5: No depression; 6-10: Mild depression; 11-15: Moderate depression; 16-20: Severe depression; and 21-27: Very severe depression. QIDS-SR-16 is often considered a more “patient-friendly” tool because it is brief and focuses on symptoms over the past seven days.

The change from baseline in QIDS-SR-16 Total score at end of treatment (EOT) was measured using an analysis of covariance (ANCOVA) in the ITT population. Post-hoc analyses measured changes using an ANCOVA in the ITT population in the 9 symptom domains of the QIDS-SR-16 score: sleep disturbance (highest score of items 1-4), sad mood, appetite/weight (highest score of items 6-9), concentration, self-criticism, suicidal ideation, interest, energy/fatigue, and psychomotor (highest score of items 15-16). The QIDS-SR-16 symptom domains with highest baseline scores were sleep disturbance (mean: lumateperone+ADT, 2.7; placebo+ADT, 2.7) and feeling sad (lumateperone+ADT, 2.6; placebo+ADT, 2.6).

The results demonstrated that, overall, lumateperone+ADT significantly improved QIDS-SR-16 Total score vs. placebo+ADT from baseline to EOT (LSMD=−2.4; effect size=−0.50; P<0.0001).

From baseline to EOT, eight of the nine QIDS-SR-16 symptom domain scores significantly improved with lumateperone+ADT vs placebo+ADT:
Sleep disturbance (LSMD, −0.5; effect size, 0.55; P<0.0001);
Sad mood (LSMD, −0.4; effect size, 0.44; P<0.0001);
Appetite/weight change (LSMD, −0.2; effect size, 0.22; P<0.05);
Concentration (LSMD, −0.2; effect size, 0.28; P<0.01);
Self-criticism (LSMD, −0.1; effect size, 0.17; P=0.065);
Suicidal ideation (LSMD, −0.1; effect size, 0.19; P<0.05);
Interest (LSMD, −0.3; effect size, 0.29; P<0.01);
Energy/fatigue (LSMD, −0.2; effect size, 0.29; P<0.01); and
Psychomotor agitation/retardation (LSMD, −0.2; effect size, 0.30; P<0.01).

These results show that in patients with MDD with inadequate ADT response, lumateperone 42 mg adjunctive to ADT significantly improved a broad range of patient-reported depressive symptoms compared with placebo, indicating that lumateperone is an effective adjunctive therapy to ADT in this setting.

### Study 5 and 6: Open-Label Extension Period

The long safety and efficacy of lumateperone was further evaluated in patients who had completed either Study 5 or Study 6 in a 26-week open-label extension for treatment with lumateperone tosylate 60 mg+ADT (NCT05061719). The primary endpoint was safety and tolerability, measured by adverse events, extrapyramidal symptoms (EPS), suicidality, and changes in laboratory parameters, vital signs, and ECG. The secondary endpoint was improvement or maintenance of improvement of MADRS Total Score and CGI-S score.

Of 809 patients enrolled and treated, 684 (84.5%) completed the treatment period. Mean MADRS Total score was 18.2 at baseline (compared to 30.7 at baseline in the double-blind Study 5/Study 6 population), and mean CGI-S was 3.4 (compared to 4.7 at baseline in the double-blind Study 5/Study 6 population). About 66% of patients were taking an SSRI as the ADT during the extension period (30% citalopram or escitalopram), about 28% were taking an SNRI (19% venlafaxine or desvenlafaxine), and 6% were taking other ADTs (primarily bupropion).

Treatment-emergent AEs (TEAEs) occurred in 68% of patients, but only 36% experienced drug-related TEAEs, and only 7% of patients discontinued treatment due to TEAEs. Similar to the Study 5 and Study 6 experience, the TEAEs reported in at least 5% of patients were minor: headache (17%), dizziness (11%), dry mouth (8%), nausea (8%), somnolence (7%), diarrhea (6%), and nasopharyngitis (5%). 99% of TEAEs reported were mild to moderate in severity. The rate of EPS-related TEAEs was about 4% with no increase in EPS scales (BARS score, AIMS, score, SAS score). Mean changes from baseline to end of treatment were minimal for body morphology (weight, BMI, waist circumference), cardiometabolic laboratory values (serum cholesterol, triglycerides, glucose, insulin, Hb A1c), prolactin levels, pulse rate, blood pressure, and ECG measures. No patients reported emergence of serious suicidal ideation or suicidal behavior during the study.

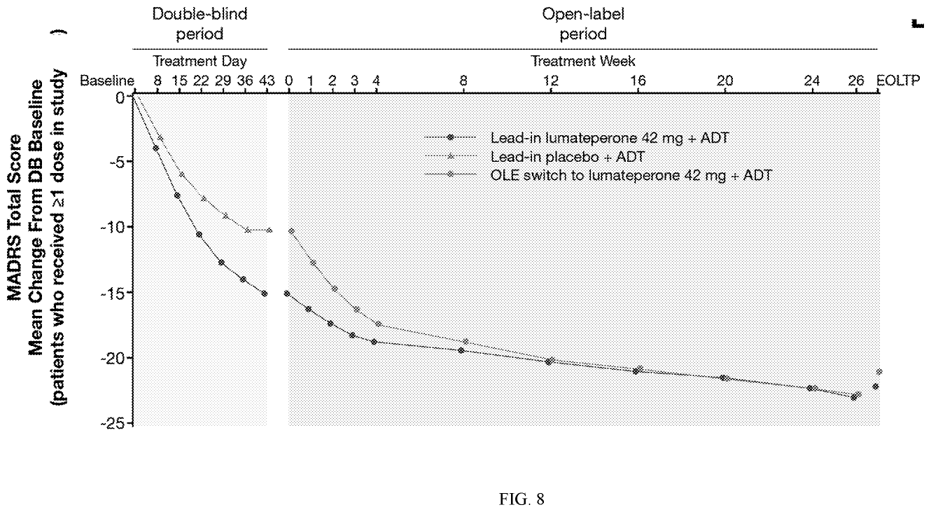

Unexpectedly, symptoms of depression continued to improve throughout the extension period, as shown by mean change from baseline to Week 26 in MADRS Total score (−22.9) and CGI-S score (−2.7). The results for MADRS Total score are shown in FIG. 8 (similar improvements were also seen in CGI-S).

At the conclusion of the extension period, 79.5% of patients were considered to have responded to treatment (≥50% decrease in MADRS Total score) and 65% of patients were considered to have remitted from disease (MADRS Total score ≤10).

These results support the long-term safety and efficacy of lumateperone tosylate 60 mg (42 mg free base) plus ADT in patients with MDD and inadequate response to ADT.

Study 5 and Study 6: Post-Hoc Analysis of MDD with Anxious Distress

A post hoc analysis of Study 5 investigated the efficacy of adjunctive lumateperone tosylate 60 mg in patients who also met DSM-5 criteria for anxious distress. Eligible adults (18-65 years) were those who had DSM-5-diagnosed MDD with inadequate response to 1 or 2 courses of antidepressant therapy (ADT) in the current depressive episode, and were experiencing a major depressive episode with an MADRS total score ≥24, a CGI-S score ≥4, and a Quick Inventory of Depressive Symptomatology-Self Report-16 item (QIDS-SR-16) score ≥14 at screening and at baseline. Inadequate response to ADT was defined as <50% improvement with ADT monotherapy for ≥6 weeks as confirmed by the Antidepressant Treatment Response Questionnaire. These patients were randomized 1:1 to a 6-week course of oral lumateperone 42 mg plus ADT or placebo plus ADT.

The primary and key secondary efficacy endpoints were change from baseline to Day 43 in MADRS Total score and CGI-S score, respectively, in the modified intent-to-treat (mITT) population, analyzed using a mixed-effects model for repeated measures (MMRM). Patient-reported outcomes included change from baseline in QIDS-SR-16 Total score, examined with an analysis of covariance, and Generalized Anxiety Disorder-7 (GAD-7) Total score, analyzed with an MMRM in the intent-to-treat population (ITT). Change in MADRS inner tension item score, response rate (≥50% MADRS Total score decrease from baseline), and remission rate (MADRS Total score ≤10) were also assessed.

The post-hoc analysis evaluated patients with DSM-5 anxious distress at screening, defined as the presence of two or more anxious symptoms (feeling tense, feeling restless, difficulty concentrating because of worry, fearful something awful may happen, or feeling out of control) during the majority of days during the current major depressive episode.

Of the 481 patients in the Study 5 mITT population, 207 (43%) had anxious distress at baseline, and none also met the criteria for mixed features or psychotic features. Baseline clinical characteristics were as follows:

| | Lumateperone + ADT | Placebo + ADT |
|---|---|---|
| | N = 109 | N = 98 |
| MADRS Total score, mean | 31.0 | 30.6 |
| CGI-S score, mean | 4.7 | 4.7 |
| QIDS-SR-16 Total score, mean | 18.8 | 18.1 |
| GAD-7 Total score, mean | 11.9 | 11.3 |

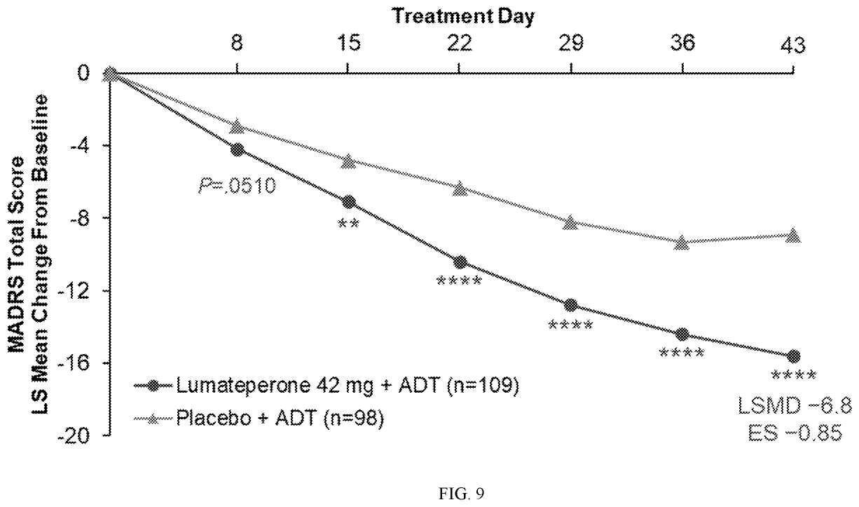

As noted previously, for the entire treatment population (mITT) in Study 5, at Day 43, there was a statistically significant improvement in MADRS Total score from baseline compared to placebo, measured as least squares mean difference (LSMD) of −4.9 (effect size −0.61). Unexpectedly, however, the results were even more profound in the patient subgroup with anxious distress. At Day 43, in the anxious distress group, the MADRS Total score was statistically significantly improved with an LSMD of −6.8 (effect size −0.85). Furthermore, a statistically significant improvement was nearly attained at Day 8 (P=0.0510), and was achieved beginning at Day 15 (P<0.01) and maintained through Day 43. The results are shown in FIG. 9.

Furthermore, in the anxious distress patients in Study 5, the MADRS Total score response rate was 52%, compared to 17% for placebo, and remission rate was 27%, compared to 11% for placebo.

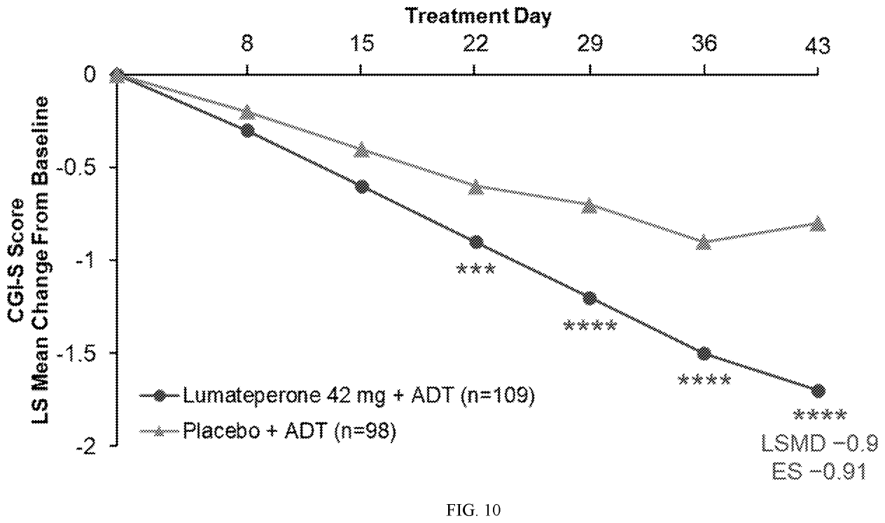

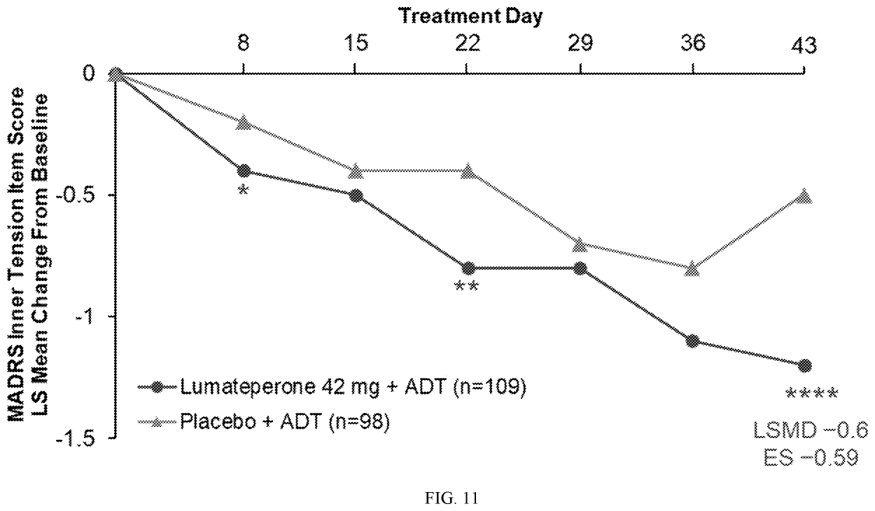

The key secondary endpoints were also achieved in the anxious distress patient group in Study 5. For the entire treatment population (mITT), at Day 43, there was a statistically significant improvement in CGI-S score from baseline compared to placebo (LSMD−0.7 (effect size −0.67), but in the anxious distress group these results were statistically stronger (LSMD−0.9 (effect size −0.91). In addition, the CGI-S score was significantly improved by Day 22, The results ae shown in FIG. 10. MADRS inner tension item score was similarly significantly improved by Day 43, as shown in FIG. 11.

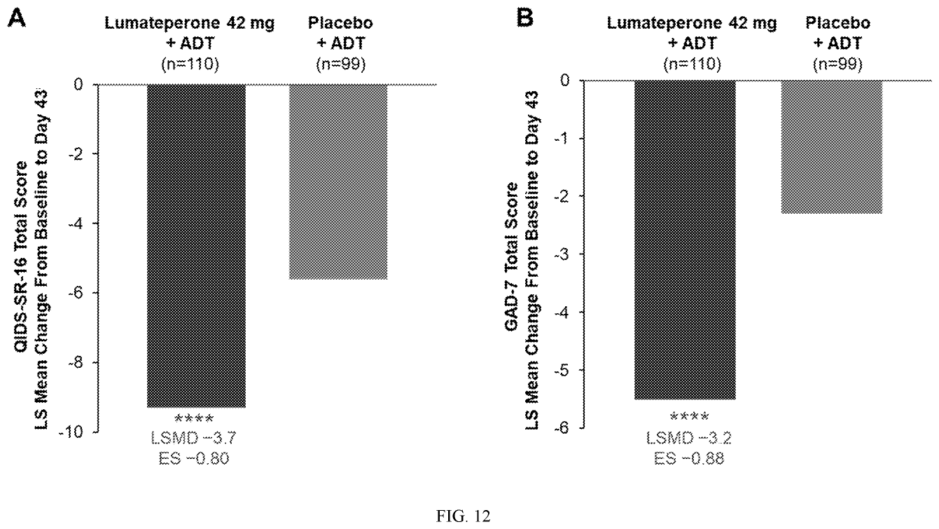

In the anxious distress patient group in Study 5, anxiety symptoms were also significantly improved in the treatment group compared to the placebo group, based on both the QIDS-SR-16 scores and the GAD-7 scores, as shown in FIGS. 12A and 12B, respectively.

As already noted, Study 6 has a substantially identical design as Study 5, and the same post-hoc analysis for patients with anxious distress was repeated for Study 6. The post hoc analysis likewise investigated the efficacy of adjunctive lumateperone tosylate 60 mg in patients who also met DSM-5 criteria for anxious distress, with the same eligibility criteria as noted above for the Study 5 post-hoc analysis. The patients were randomized 1:1 to a 6-week course of oral lumateperone 42 mg plus ADT or placebo plus ADT. The primary and key secondary efficacy endpoints and patient-reported outcomes were the same as noted for the post-hoc analysis of Study 5.

Of the 469 patients in the Study 6 mITT population, 179 (38%) had anxious distress at baseline. Baseline clinical characteristics were as follows:

| | Lumateperone + ADT | Placebo + ADT |
|---|---|---|
| | N = 88 | N = 91 |
| MADRS Total score, mean | 30.9 | 31.9 |
| CGI-S score, mean | 4.6 | 4.6 |
| QIDS-SR-16 Total score, mean | 17.9 | 18.2 |

As noted previously, and very similar to Study 5, for the entire treatment population (mITT) in Study 6, at Day 43, there was a statistically significant improvement in MADRS Total score from baseline compared to placebo, measured as least squares mean difference (LSMD) of −4.5 (effect size −0.56). At Day 43, in the anxious distress group, the MADRS Total score was statistically significantly improved with an LSMD of −4.7 (effect size −0.59). Furthermore, a statistically significant improvement was attained at Day 22 (P<0.05), and was maintained through Day 43. In the anxious distress patients in Study 6, the MADRS Total score response rate was 49%, compared to 23% for placebo, and remission rate was 28% compared to 11% for placebo.

The key secondary endpoints were also achieved in the anxious distress patient group in Study 6. For the entire treatment population (mITT), at Day 43, there was a statistically significant improvement in CGI-S score from baseline compared to placebo (LSMD−0.5 (effect size −0.51), with similar results in the anxious distress group (LSMD−0.5 (effect size −0.52). In addition, the CGI-S score was significantly improved by Day 22 (P<0.05). MADRS inner tension item score was similarly significantly improved by Day 43 (P<0.05).

In the anxious distress patient group in Study 6, anxiety symptoms were also significantly improved in the treatment group compared to the placebo group, based on the QIDS-SR-16 scores (LSMD−2.2, (effect size −0.45)).

### Study 5 and Study 6: Post-Hoc Analysis of Anhedonia in MDD

A post hoc analysis of Study 5 investigated the efficacy of lumateperone tosylate 60 mg in improving anhedonia, assessed by the MADRS anhedonia factor score, in patients with MDD, including patients with significant anhedonia at baseline. As described above, the primary and key secondary efficacy endpoints in Study 5 were change from baseline to Day 43 in MADRS Total score and CGI-S score, respectively, in the modified intent-to-treat (mITT) population, analyzed using a mixed-effects model for repeated measures (MMRM).

The MADRS anhedonia factor score is based on six MADRS item components, apparent sadness, reported sadness, concentration difficulties, lassitude, and inability to feel. Each item is scored on a scale of 0-6, and the resulting MADRS anhedonia factor score ranges from 0-30. Scores of 0-6 are considered to represent no anhedonia, 7-12 is mild anhedonia, 13-18 is moderate anhedonia, and 19-30 is severe anhedonia. In the Study 5 mITT patient population, the baseline mean MADRS anhedonia factor score was 18.4 in the lumateperone group and 18.2 in the placebo group. The most prominent MADRS single items at baseline were reported sadness and apparent sadness.

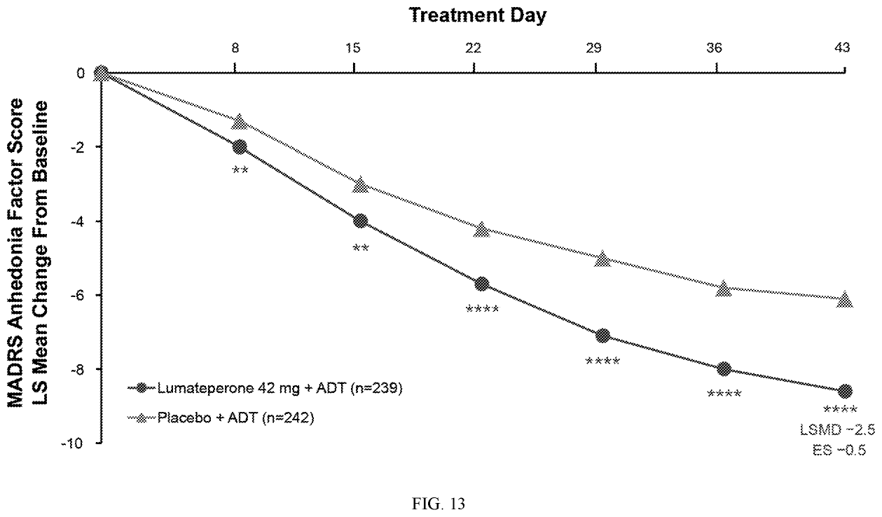

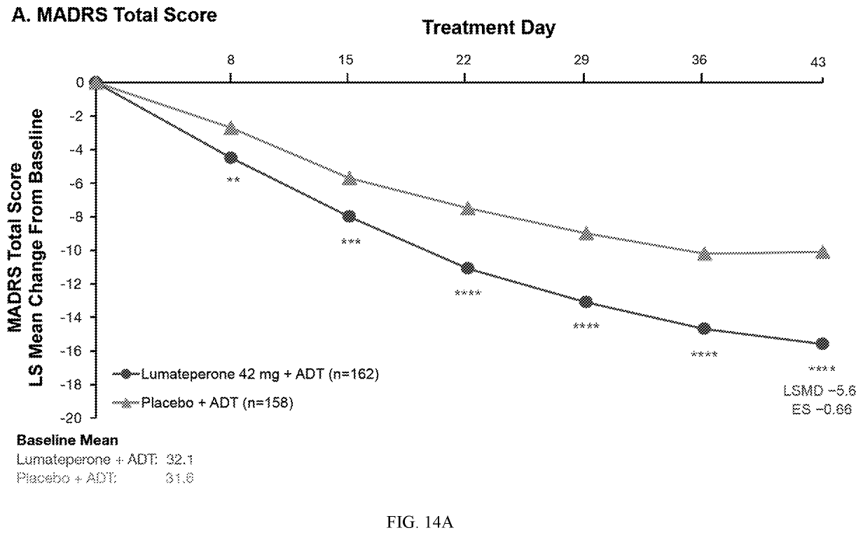

It was unexpectedly found that lumateperone+ADT significantly improved the MADRS anhedonia factor score compared with placebo+ADT at every patient visit, from Day 8 through Day 43 in Study 5. The results are shown in FIG. 13. Each of the 5 items comprising the MADRS anhedonia factor score also significantly improved in the lumateperone+ADT group by Day 29, with continued improvement through Day 43.

Of the 481 patients in the Study 5 mITT population, 320 had a higher MADRS anhedonia factor score at baseline than the mITT median (median mITT MADRS anhedonia factor score was 18), This patient subpopulation had a baseline mean MADRS Total score of 31.9 (lumateperone group mean 32.1, N=162; placebo group mean 31.6, N=158), and a baseline mean MADRS anhedonia factor score of 19.5 (mean 19.5 in both lumateperone and placebo groups).

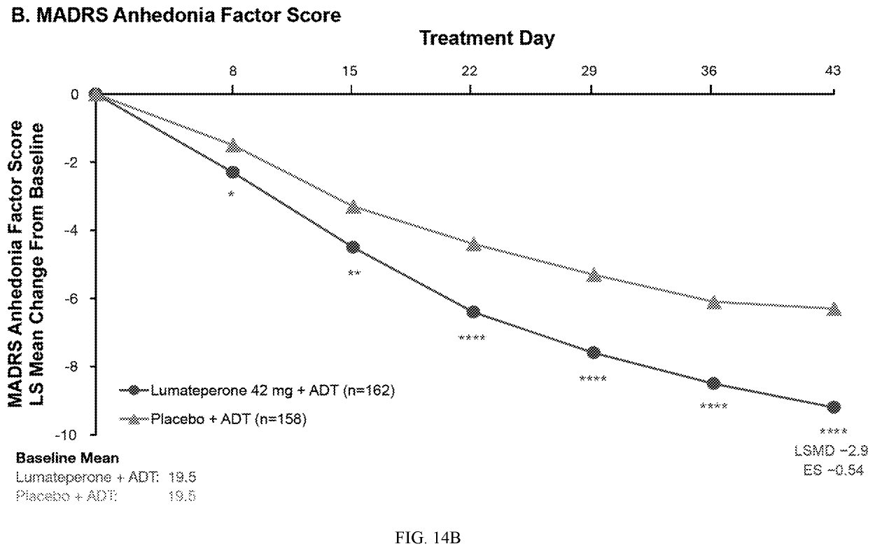

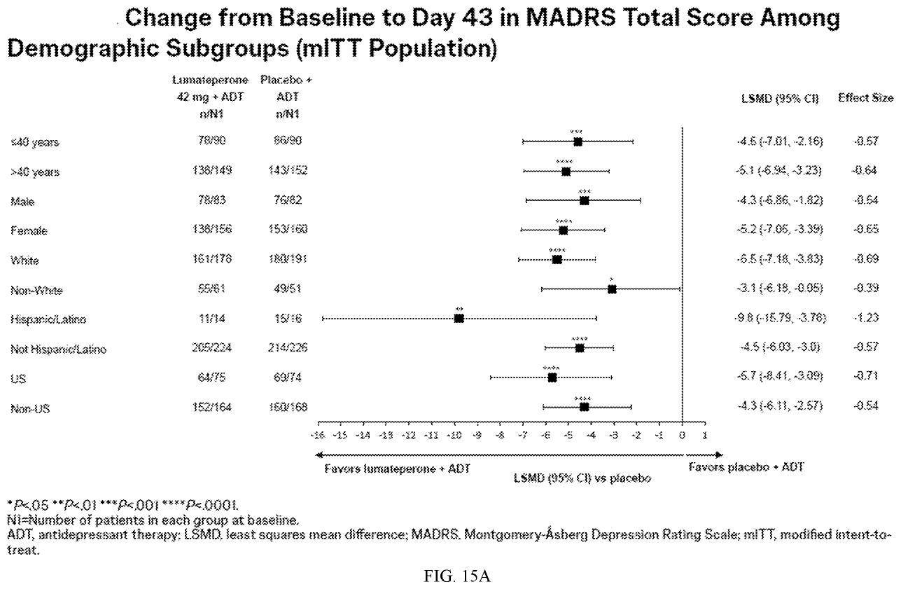

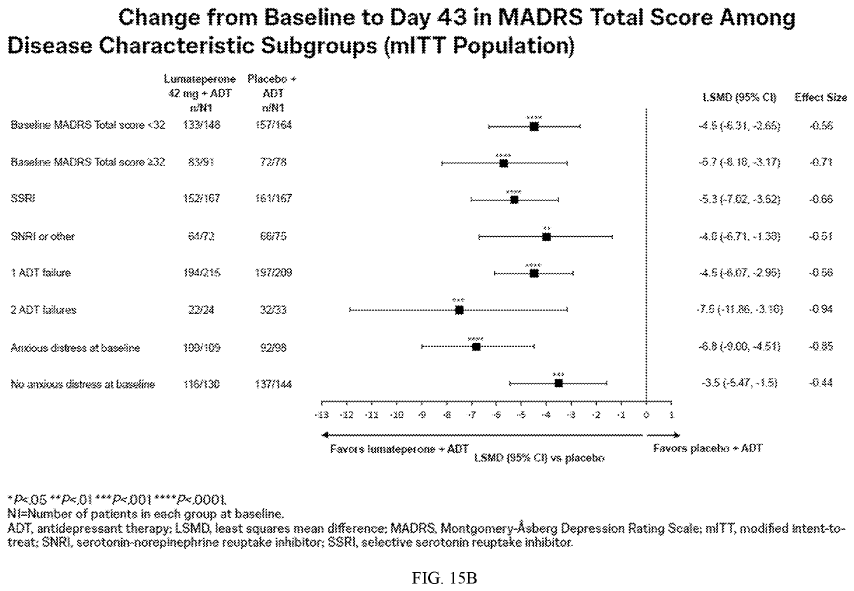

It was also unexpectedly found that in this high baseline anhedonia subgroup, lumateperone+ADT significantly improved both the MADRS Total Score and the MADRS anhedonia factor score compared with placebo+ADT at every patient visit, from Day 8 through Day 43. The results are shown in FIG. 14A and FIG. 14B.

The same post-hoc analysis for anhedonia was performed for Study 6, as described above for Study 5. In the Study 6 mITT patient population, the baseline mean MADRS anhedonia factor score was 19.0 in the lumateperone group and 19.3 in the placebo group. The most prominent MADRS single items at baseline were reported sadness and apparent sadness.

Like Study 5, in Study 6, it was found that lumateperone+ADT significantly improved the MADRS anhedonia factor score compared with placebo+ADT, but only beginning at Day 22. Each of the 5 items comprising the MADRS anhedonia factor score also significantly improved in the lumateperone+ADT group by Day 29, with continued improvement through Day 43.

Of the 469 patients in the Study 6 mITT population, 281 had a higher MADRS anhedonia factor score at baseline than the mITT median (median mITT MADRS anhedonia factor score was 19), This patient subpopulation had a baseline mean MADRS Total score of 33.1 (lumateperone group mean 32.9, N=129; placebo group mean 33.3, N=152), and a baseline mean MADRS anhedonia factor score of 20.9 (mean 20.9 in both lumateperone and placebo groups).

Similar to Study 5, it was found that in this high baseline anhedonia subgroup, lumateperone+ADT significantly improved the MADRS Total Score at every patient visit, from Day 8 through Day 43, and the MADRS anhedonia factor score compared with placebo+ADT from Day 22 through Day 43.

### Study 5: Post-Hoc Analysis of Patient Subgroups

Demographic and clinical characteristics can influence treatment outcomes in patients with MDD, making it essential to evaluate treatment efficacy in distinct subgroups to establish consistency of treatment effect. An analysis was therefore conducted of the efficacy of lumateperone 42 mg (60 mg lumateperone tosylate) adjunctive to ADT across demographic and clinical subgroups of patients with MDD with inadequate ADT response (prior treatment).

As noted above, Study 5 enrolled eligible adults (18-65 years of age) who met DSM-5 criteria for MDD with inadequate response to 1-2 courses of prior ADT in the current depressive episode, where inadequate response was defined as less than 50% improvement on the Antidepressant Treatment Response Questionnaire. Patients were experiencing a major depressive episode (MADRS Total score ≥24 and CGI-S score ≥4) and had a QIDS-SR-16 item score ≥14 at screening and baseline. Patients were randomized 1:1 to 6-weeks of treatment with lumateperone plus ADT or placebo plus ADT, and the primary and key secondary endpoints were the changes from baseline to Day 43 in MADRS Total score and CGI-S score, respectively. MADRS response rate and remission rate, and change from baseline to Day 43 in QIDS-SR-16 Total Score was also assessed.

The mITT population comprised 481 patients (239 in treatment group, 242 in placebo group). Efficacy was evaluated in the overall population and in patient subgroups by demographic and baseline disease characteristics using a mixed-effects model for repeated measures (MMRM) or analysis of covariance. The following patient characteristics were considered:

| | Baseline Disease/Characteristic |
|---|---|
| Demographic Subgroups | Subgroups |
| Age: ≤40 years or >40 years | Severity: baseline MADRS score <32 |
| Sex: Male or female | or ≥32 |
| Race: White or non-white | ADT: SSRI or SNRI/Other |
| Ethnicity: Hispanic/Latino or | ADT Failures in Current Episode: 1 or 2 |
| not Hispanic/Latino | DSM-5 Anxious Distress: present or |
| Geography: U.S. or non-U.S. | absent |

Overall, the patient mean age was 45 years, about 77% of patients were White (about 23% non-White), about 66% were female (about 34% male), and about 50% were Hispanic or Latino. About 90% of patients had one ADT failure in the current episode, and about 10% had two ADT failures. About 66% of patients received treatment with adjunct SSRIs, and the remaining patients were treated with SNRIs or bupropion (about 28% SNRI, and about 6% bupropion). All characteristics were similar between the treatment and placebo groups.

In the full mITT population, both the primary and secondary endpoints were met: LSMD versus placebo of −4.9 (effect size −0.61; P<0.0001) for MADRS Total Score, and LSMD versus placebo of −0.7 (effect size −0.67; P<0.0001) for CGI-S score. There was also significant improvement in QIDS-SR-16 Total Score in the ITT population (LSMD versus placebo −2.4 (effect size −0.61)).

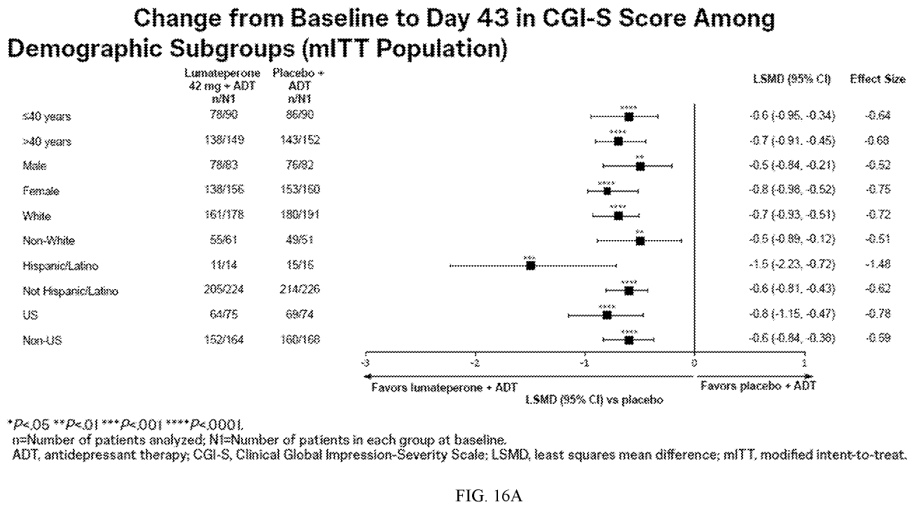

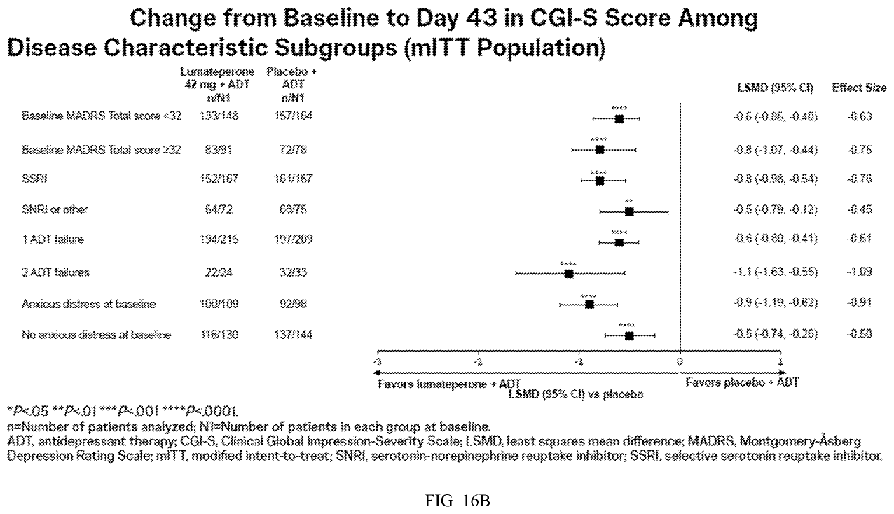

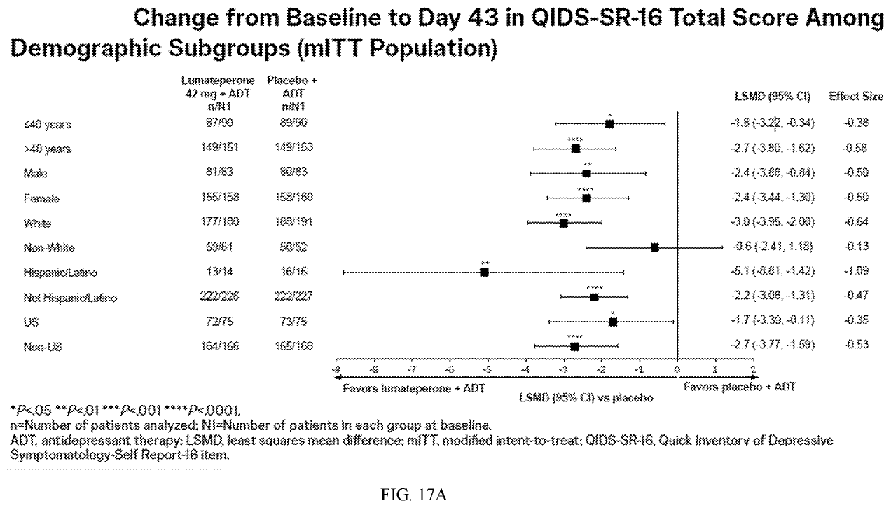

Subgroup Analyses are summarized in FIGS. 15A and 15B. The results demonstrate consistent significant improvements in the lumateperone plus ADT group compared to placebo plus ADT group for MADRS Total Score at Day 43 in all demographic subgroups assessed (FIG. 15A). Furthermore, lumateperone plus ADT significantly improved MADRS Total Score at Day 43 irrespective of baseline disease characteristics assessed (FIG. 15B). Corresponding results were observed for the secondary endpoint, CGI-S score (see FIGS. 16A and 16B).

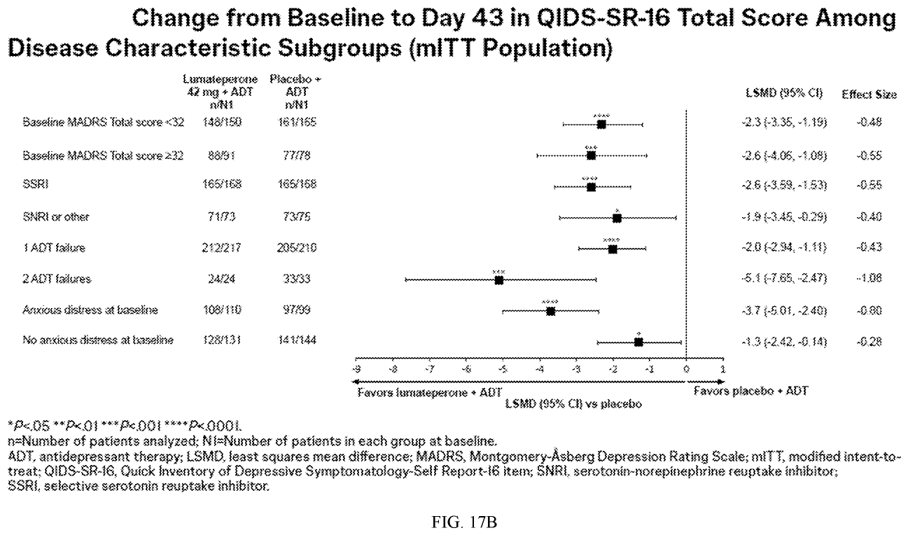

Lumateperone plus ADT significantly improved QIDS-SR-16 Total Score at Day 43 in all patient subgroups assessed, except for race, in which the results did not reach statistical significance in non-Whites (see FIGS. 17A and 17B).

The effects on MADRS response rate and remission rate were more variable across patient subgroups compared to the raw MADRS Total scores. It was unexpectedly found that treatment with lumateperone plus ADT consistently led to statistically significant improvements (P<0.05) in MADRS response at Day 43 versus placebo plus ADT in all patient demographic subgroups, except for race (White, P<0.0001; non-White, P=0.2660) and ethnicity (Hispanic/Latino, P=0.0938; not Hispanic/Latino, P<0.0001).

Treatment with lumateperone plus ADT consistently led to statistically significant improvements (P<0.05) in MADRS remission at Day 43 vs placebo plus ADT across subgroups for age, sex, geographic region, disease severity, and presence/absence of anxious distress, but not for race (White, P=0.0002; non-White, P=0.7936), ethnicity (Hispanic/Latino, P=0.1009; not Hispanic/Latino, P=0.0028), type of ADT (SNRI/other, P=0.3141; SSRI, P=0.0010), or number of ADT failures in the current episode (2 failures, P=0.2127; 1 failure, P=0.0023).

Most of these differences, especially regarding race and ethnicity, may be attributed to wide error ranges and are likely not clinically important. However, these results suggest that treatment with lumateperone plus ADT may be particularly beneficial in improving remission in patients who are being treated with SSRI therapy compared to SNRI or other antidepressant therapy (e.g., bupropion).

### Study 6: Post-Hoc Analysis of Patient Subgroups

Similar to the analysis described above for Study 5, an analysis was conducted of the results of Study 6 across demographic and clinical subgroups of patients with MDD with inadequate ADT response (prior treatment).

As with Study 5, Study 6 enrolled eligible adults (18-65 years of age) who met DSM-5 criteria for MDD with inadequate response to 1-2 courses of prior ADT in the current depressive episode (as defined above), and who were experiencing a major depressive episode (MADRS Total score ≥24 and CGI-S score ≥4) and had a QIDS-SR-16 item score ≥14 at screening and baseline. The mITT population comprised 469 patients (232 in treatment group, 237 in placebo group). Efficacy was evaluated in the overall population and in patient subgroups by demographic and baseline disease characteristics using a mixed-effects model for repeated measures (MMRM) or analysis of covariance. The same patient characteristics (demographic subgroups and baseline/disease characteristic subgroups were used as described above for the Study 5 post-hoc analysis.

Lumateperone+ADT significantly improved MADRS Total score from baseline to Day 43 in the following demographic subgroups versus placebo+ADT:
Age (≤40 y: LSMD=−4.3; P<0.01; >40 y: LSMD=−4.6; P<0.0001);
Sex (male: LSMD=−4.0; P<0.01; female: LSMD=−4.8; P<0.0001);
Region (US: LSMD=−3.5; P<0.05; non-US: LSMD=−4.8; P<0.0001).

The treatment effect, however, did not reach statistical significance for race (White: LSMD=−4.5; P<0.0001; non-White: LSMD=−0.7; P=0.8717) and ethnicity (Hispanic/Latino: LSMD=−2.9; P=0.1468; not Hispanic/Latino: LSMD=−4.8; P<0.0001) subgroups.

MADRS Total score significantly improved from baseline to Day 43 with lumateperone+ADT in the following clinical subgroups versus placebo+ADT:
Baseline disease severity (MADRS score <32: LSMD=−4.6; P<0.0001; MADRS score ≥32: LSMD=−4.4; P<0.001);
Type of ADT (SSRI: LSMD=−4.3; P<0.0001; SNRI and other: LSMD=−4.6; P<0.001),
Number of ADT failures in the current episode (1 ADT failure: LSMD=−4.4; P<0.0001; 2 ADT failures: LSMD=−5.1; P<0.05); and
Presence of anxious distress (yes: LSMD=−4.7; P<0.001; no: LSMD=−4.4; P<0.0001).

In addition, lumateperone+ADT significantly improved (P<0.05) CGI-S and QIDS-SR-16 Total scores from baseline to Day 43 versus placebo+ADT across most subgroups.

These results show that lumateperone+ADT demonstrated robust efficacy versus placebo+ADT in subgroups of patients with MDD, supporting the drug as a promising adjunctive treatment option for patients with MDD with inadequate ADT response.

### Study 5 and Study 6: Post-Hoc Analysis of Onset and Duration of Treatment-Emergent Adverse Events

Standard antidepressant therapy (ADT), such as selective serotonin reuptake inhibitors and serotonin-norepinephrine reuptake inhibitors, are used to treat MDD, but are often limited by tolerability concerns. Adverse effects associated with ADTs (e.g., sexual dysfunction, weight gain, and sleep disturbances) frequently reduce treatment adherence. Given the limited efficacy and tolerability of standard ADTs, alternative strategies are needed.

In studies across several disease indications (schizophrenia, bipolar disorder, and MDD), lumateperone (e.g., lumateperone tosylate) has consistently been found to be well-tolerated. Compared to other agents in the antipsychotic class, lumateperone has shown minimal to low risk of extrapyramidal symptoms, weight gain, prolactin increase, cardiometabolic side effects, or other side effects.

This pooled analysis of results from Study 5 and 6 aimed to evaluate the timing and duration of treatment-emergent adverse events in patients treated with lumateperone 42 mg (60 mg lumateperone tosylate) adjunctive to ADT in patients with MDD with inadequate ADT response (prior treatment).

As noted above, Studies 5 and 6 enrolled eligible adults (18-65 years of age) who met DSM-5 criteria for MDD with inadequate response to 1-2 courses of prior ADT. The combined pooled safety population comprised 964 patients, 483 in the lumateperone plus ADT groups, and 481 in the placebo plus ADT groups. Overall, about 91% patients completed the 6-week course of treatment.

Treatment-emergent adverse events (TEAEs) occurred in about 68% of patients in the treatment group (considered treatment-related in 50% of patients), and in about 45% patients in the placebo group (considered treatment-related in 20% of patients). Only dizziness, dry mouth, somnolence, nausea, fatigue, and diarrhea, were observed in more than 5% of treatment group patients and at more than twice the rate of placebo. Serious TEAEs occurred in only one patient in each group (about 0.2%) and was not related to treatment (polypectomy in the treatment group patient, joint dislocation in the placebo group patient). No patients died during the treatment period.

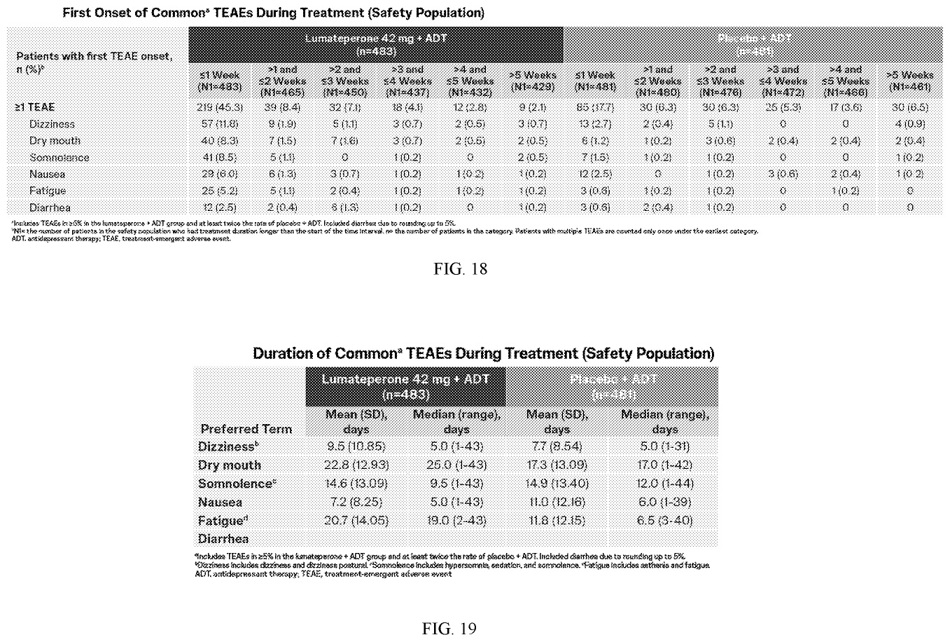

The timing of first onset of TEAEs is summarized in FIG. 18. Overall, TEAE onset occurred during the first week of treatment for most patients. However, while about 18% of placebo group patients experienced TEAE onset during the first week of treatment, 45% of lumateperone group patients experienced TEAE onset during the first week of treatment. Interestingly, in the lumateperone group, the rate of first TEAE onset steadily decreased after Week 1, but in the placebo group, the rate of first TEAE decreased from Week 1 to Week 4, then increased.

Duration of TEAEs is summarized in FIG. 19. The mean duration of somnolence and dizziness were similar between the two treatment groups. Unexpectedly, however, in the lumateperone group, the mean duration of nausea was shorter (7.2 days) while the mean durations of fatigue (20.7 days) and dry mouth (22.8 days) were somewhat longer than those observed in the placebo group (nausea: 11.0 days; fatigue: 11.8 days; dry mouth: 17.3 days).

All TEAEs tended to resolve within 1-3 weeks of the onset of treatment. These results further demonstrate the safety and tolerability of lumateperone treatment adjunctive to ADT.

### Study 5 and Study 6 and Open-Label Extension Period: Short-term and Long-Term Remission Rates

In patients with MDD, remission rates are low following first-line treatment, typically only about 25%, and this further declines with subsequent treatments. Failure to achieve remission is associated with reduced quality of life and increased relapse rates, underscoring the importance of remission as a key goal of MDD treatment.

This pooled analysis of results from Study 5 and 6, as well as the combined open-label extension period, aimed to evaluate the rate of MADRS Total Score remission in patients treated with lumateperone 42 mg (60 mg lumateperone tosylate) adjunctive to ADT in patients with MDD with inadequate ADT response (prior treatment). MADRS remission is defined as achieving an MADRS Total score of 10 or less, and MADRS complete remission is defined as achieving an MADRS Total score of 5 or less. Remission was evaluated at each patient visit, and sustained remission occurs when a status of MADRS remission is achieved at consecutive visits (MADRS Total score 10 or less).

The pooled mITT population from Studies 5 and 6 totaled 950 patients (471 in the lumateperone plus ADT group, and 479 in the placebo plus ADT group). 809 patients from Study 5 and 6 further enrolled in the open-label extension period.

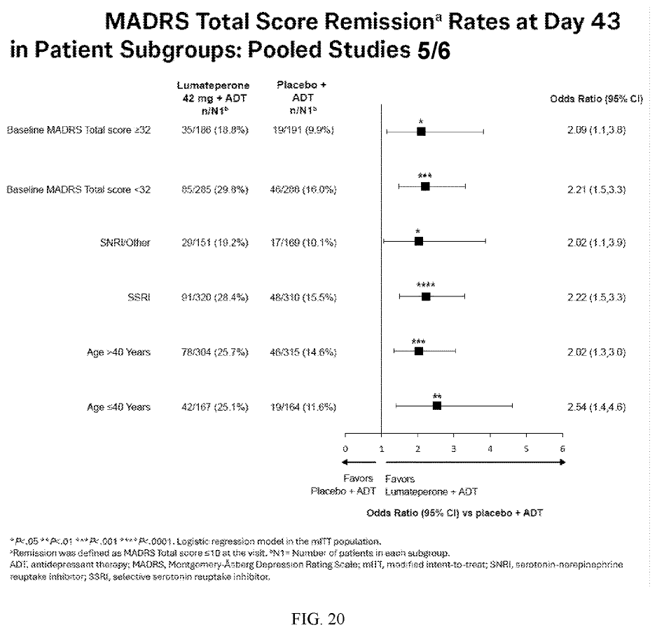

At Day 43 in the pooled Study 5/6 results, the rate of MADRS Total score remission was about 26% in the lumateperone group, compared to only about 14% in the placebo group, a statistically significant result (P<0.0001). In an analysis of subgroup results, significantly greater MADRS remission rate was observed for lumateperone-treated patients in all subgroups assessed. See FIG. 20. Similarly, the rate of MADRS Total Score complete remission was 10.6% in the lumateperone group, compared to only 5.6% in the placebo group (P <0.01).

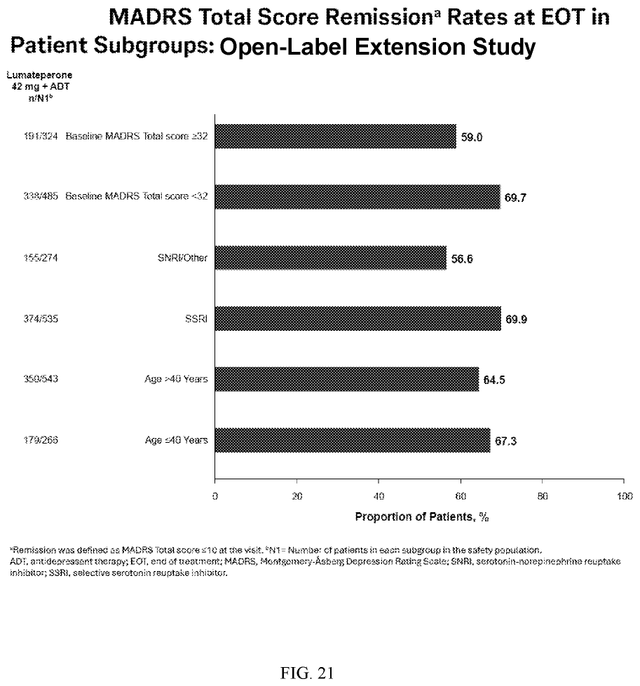

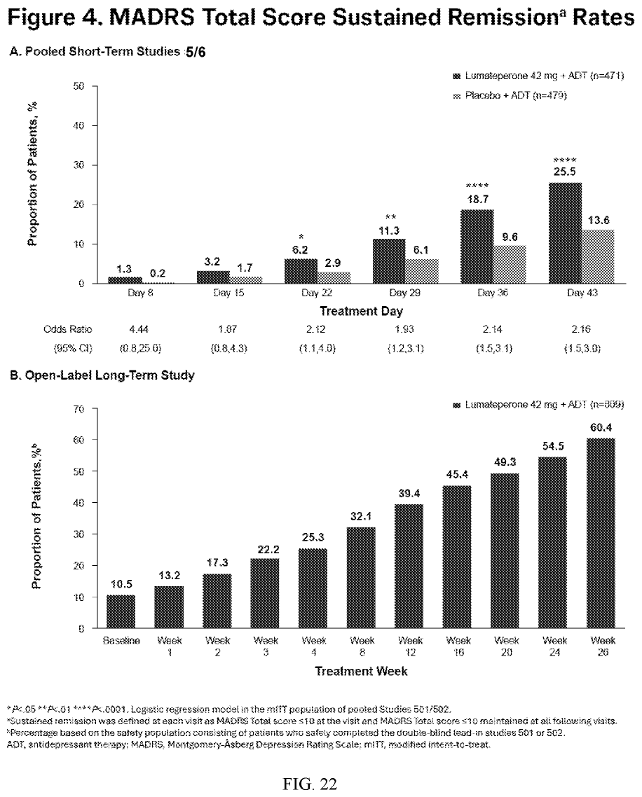

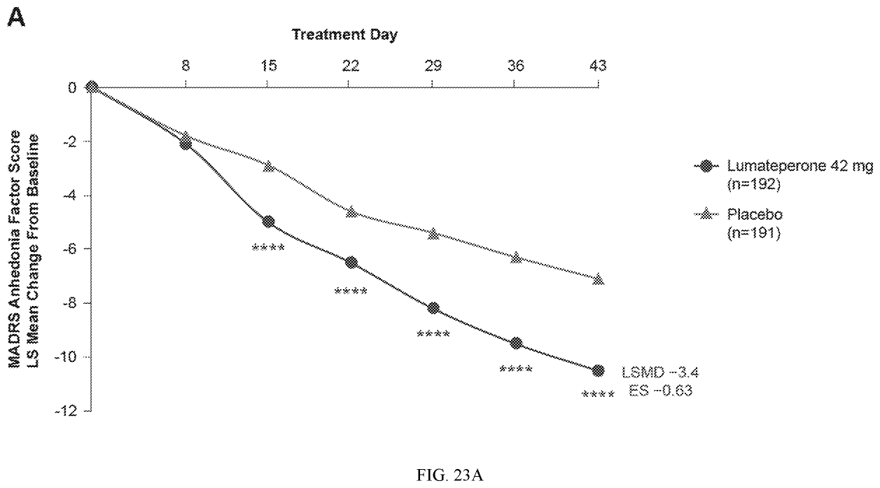

At the end of the 26-week open-label extension period, the MADRS Total Score remission rate increased to 65%, and similar results were observed in each of the subgroup analyses (at least 55% remission rate in each subgroup). See FIG. 21. Similarly, the rate of MADRS Total Score complete remission increased to 44% at the end of the 26-week open-label extension period.

Beginning at Day 22 in the pooled Study 5/6 results, the rate of MADRS Total score sustained remission was significantly greater in the lumateperone plus ADT group compared to the placebo plus ADT group (P<0.05 at Day 22; P<0.01 at Day 29; and P<0.0001 at Days 36 and 43). See FIG. 22A. In the open-label extension period, the rate of sustained remission increased from about 10% at baseline to 60% by Week 26. See FIG. 22B.

These results demonstrate that treatment with lumateperone 42 mg (60 mg lumateperone tosylate) adjunctive to ADT in patients with MDD with inadequate ADT response provides significant improvements in MADRS remission rate compared to placebo plus ADT, and that this result is sustained long-term, resulting in remission in nearly 2 out of 3 patients within 6-months of the beginning of treatment.

### Study 5 and Study 6: Post-Hoc Analysis of Metabolic Profile

A pooled analysis of Study 5 and 6 evaluated the metabolic profile associated with the treatment regimen. The safety population comprised 964 patients (lumateperone+ADT, n=483; placebo+ADT, n=481). Subgroups were analyzed by baseline body mass index (BMI) category (normal weight: ≥18.5 to <25 kg/m²; overweight: ≥25 to <30 kg/m²; and obese: ≥30 kg/m²).

Changes from baseline to end of treatment (EOT) for weight, BMI, and waist circumference were minimal in the lumateperone+ADT group compared to the placebo+ADT group: for weight change, −0.1 kg vs. +0.0 kg, for lumateperone and placebo, respectively; for BMI change, −0.0 kg/m² vs. +0.0 kg/m², for lumateperone and placebo, respectively; and for waist circumference, −0.2 cm vs. −0.3 cm, for lumateperone and placebo, respectively.

Potentially clinically significant weight increase (≥7% from baseline) was rare in both treatment groups, but also substantially higher in the placebo group than the lumateperone group: lumateperone+ADT, 0.4%; placebo+ADT, 1.3%. There were no clinically meaningful changes in cardiometabolic parameters or prolactin levels.

At baseline, the proportions of patients across the three BMI categories were similar in each group:
Normal weight: lumateperone+ADT, 32% vs. placebo+ADT, 34%;
Overweight: lumateperone+ADT, 39% vs. placebo+ADT, 32%; and
Obese: lumateperone+ADT, 29% vs. placebo+ADT, 34%.

Changes from baseline to end of treatment (EOT) for weight, BMI, and waist circumference were minimal across the BMI subgroups and treatment groups: for weight, −0.3 kg to +0.3 kg; for BMI, −0.1 kg/m² to +0.1 kg/m²); for waist circumference, −0.6 cm to +0.1 cm.

Potentially clinically significant weight increase (≥7% from baseline) was rare in both treatment groups, but also substantially higher in the obese lumateperone group than the obese placebo group:
Normal weight: lumateperone+ADT, 0.7% vs. placebo+ADT, 1.2%;
Overweight: lumateperone+ADT, 0.6% vs. placebo+ADT, 0%;
Obese: lumateperone+ADT, 0% vs. placebo+ADT, 2.5%.

Changes in cardiometabolic parameters and prolactin levels at EOT were not clinically relevant across BMI subgroups and treatment groups. Most patients remained in their BMI category by EOT (normal weight: lumateperone+ADT, 91.0% vs. placebo+ADT, 95.2%; overweight: 89.8% vs. 86.9%; obese: 91.5% vs. 94.5%).

These results support that lumateperone+ADT had a favorable safety profile with minimal changes in metabolic parameters across BMI subgroups, indicating that lumateperone is a well-tolerated adjunctive treatment for patients with MDD with inadequate ADT response.

Study 7 and Post-Hoc Analysis: Anhedonia in MDD with Mixed Features
Study 7: MDD and BPD with Mixed Features

Study 7 (NCT04285515) was a randomized, double-blind, placebo-controlled trial of lumateperone 42 mg (60 mg lumateperone tosylate) administered to patients with MDD diagnosed according to DSM-5 with mixed features, and bipolar I or bipolar II depression with mixed features.

The Diagnostic and Statistical Manual of Mental Disorders 5 (DSM-5) and 5-text revision (DSM-5-TR) define mixed features in MDD or bipolar depression (BPD) as depressive episodes having 3 or more manic or hypomanic symptoms during the majority of days of a major depressive episode (MDE) without meeting the criteria for mania or hypomania. Mixed features are common in MDD and BPD (25%-35%) and patients with mixed features have more severe symptoms, more comorbidities, increased suicide risk, and poorer treatment response than patients without mixed features

Eligible patients were adults aged 18-75 experiencing a current MDE (MADRS score of at least 24 and CGI-S score of at least 4) and having a Young Mania Rating Scale (YMRS) score of 4-16 (inclusive). Patients were stratified by MDD versus BPD, and were randomized 1:1 for 6-weeks of treatment.

The primary and key secondary efficacy endpoints were change from baseline to Day 43 in MADRS Total score and CGI-S score, respectively, analyzed using a mixed-effects model for repeated measures. Three populations with mixed features were assessed: the overall combined MDD and BPD population, the individual MDD population, and the individual BPD population. Safety assessments included adverse events (AEs), laboratory parameters, vital signs, and extrapyramidal symptoms.

385 patients received treatment (placebo, n=193; lumateperone, n=192) and 344 (89.4%) completed the study. The mITT population (N=383) was evenly split between MDD and BPD (48% to 52%). The patient diagnoses were as follows:

| | Lumateperone | Placebo |
|---|---|---|
| Total | N = 192 | N = 193 |
| MDD with mixed features | N = 92 | N = 93 |
| BPD I with mixed features | N = 78 | N = 79 |
| BPD II with mixed features | N = 22 | N = 21 |

Baseline patient parameters were as follows:

| | MDD + BPD | | MDD | | BPD (I + II) | |
|---|---|---|---|---|---|---|
| | Luma | Placebo | Luma | Placebo | Luma | Placebo |
| MADRS Total | 31.3 | 31.1 | 30.8 | 31.2 | 31.1 | 31.8 |
| score, mean | | | | | | |
| CGI-S score, | 4.5 | 4.5 | 4.4 | 4.4 | 4.6 | 4.6 |
| mean | | | | | | |
| YMRS Total | 9.0 | 9.2 | 9.3 | 9.3 | 8.7 | 9.1 |
| score, mean | | | | | | |

The results showed that patients with MDD or BPD and mixed features treated with lumateperone 42 mg had significantly greater MADRS Total score improvement compared with placebo as indicated by mean change from baseline to Day 43 (LSMD−5.7; effect size −0.64). Improvements with lumateperone were also significant in individual patient populations with MDD with mixed features (LSMD−5.9; effect size −0.67) and BPD with mixed features (LSMD−5.7; effect size −0.64). In the combined population as well as in each subpopulation, MADRS total score results were significant beginning on Day 15 (P<0.0001 for combined and MDD alone; P<0.001 for BPD)) and remained significant for the remainder of the study. For some MADRS measures, results were significant at Day 8 (apparent sadness, reduced sleep; P<0.05).

Significant improvements compared with placebo were also observed for CGI-S, the key secondary endpoint, in the combined MDD and BPD population (LSMD−0.6; effect size −0.59), individual MDD population (LSMD−0.6; effect size −0.57), and individual BPD population (LSMD−0.6 effect size −0.61). Lumateperone treatment was generally safe and well tolerated and consistent with prior studies. The most common treatment-emergent AEs with lumateperone (≥5% and twice placebo) were somnolence, dizziness, and nausea. No serious AEs were reported with lumateperone treatment.

### Study 7: Post-Hoc Analysis for Anhedonia

The post-hoc analysis of Study 7 investigated the efficacy of lumateperone tosylate 60 mg in improving anhedonia, assessed by the MADRS anhedonia factor score, in patients with MDD or bipolar depression with mixed features. All three populations were analyzed (combined, MDD, and BPD) using a mixed-effects model for repeated measures (MMRM) in the mITT population.

The MADRS anhedonia factor score is based on six MADRS item components, apparent sadness, reported sadness, concentration difficulties, lassitude, and inability to feel. Each item is scored on a scale of 0-6, and the resulting MADRS anhedonia factor score ranges from 0-30. Scores of 0-6 are considered to represent no anhedonia, 7-12 is mild anhedonia, 13-18 is moderate anhedonia, and 19-30 is severe anhedonia. At baseline, in all 3 populations, the most prominent individual items of the MADRS anhedonia factor score were reported sadness and apparent sadness.

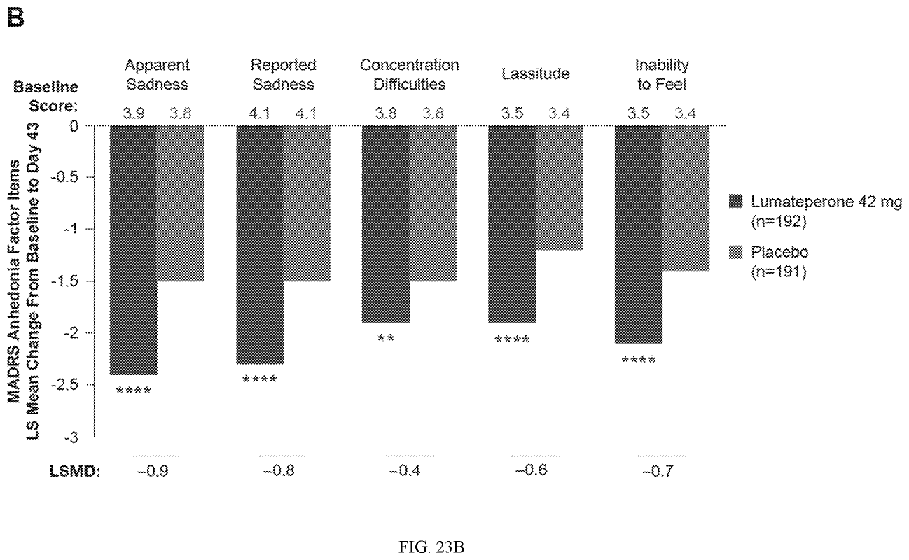

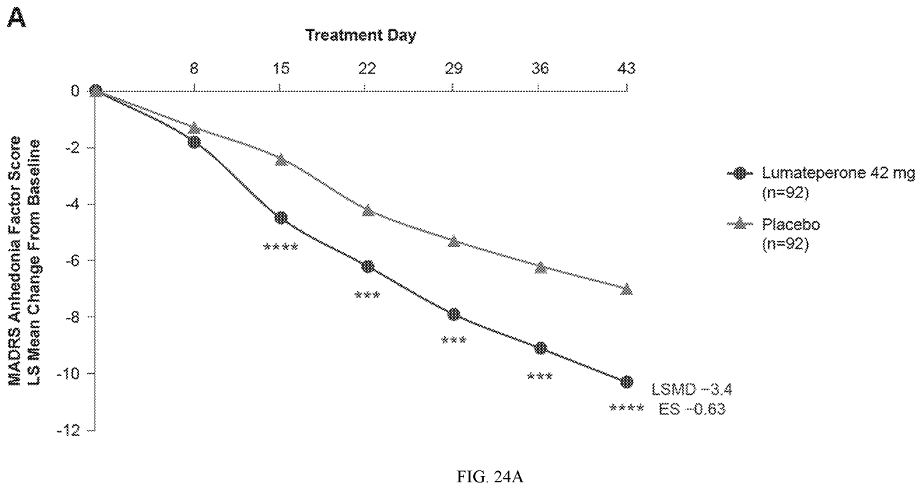

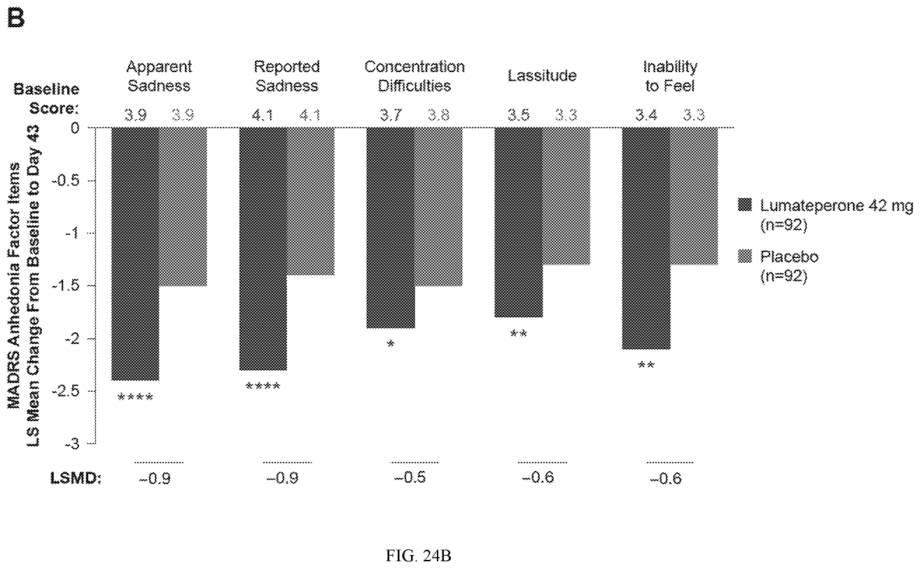

Lumateperone treatment significantly improved the MADRS anhedonia factor score from baseline to Day 43 compared to placebo (LSMD−3.4, effect size −0.63) in the combined MDD/BPD population and in the MDD population (LSMD−3.4, effect size −0.63). Each of the individual MADRS anhedonia factor score items was also significantly improved at Day 43 (LSMD−0.4 to −0.9). In the combined population, results were significant (P<0.05) beginning on Day 8 for apparent sadness, and beginning on Day 15 for reported sadness, lassitude, and inability to feel, and beginning on Day 22 for concentration difficulties. In the MDD population, results were significant (P<0.05) beginning on Day 15 for apparent sadness, reported sadness, and lassitude, and beginning on Day 22 for concentration difficulties and inability to feel. Substantial improvements in each item, and the anhedonia factor score, continued throughout the study. The results are shown in FIGS. 23A and 23B (combined population) and in FIGS. 24A and 24B (MDD population). Results in the BPD subpopulation were similar to the MDD subpopulation (e.g., MADRS total score mean change from baseline to Day 43, LSMD−3.3, effect size −0.61),

Lumateperone treatment also significantly improved YMRS score from baseline to Day 43 compared to placebo (LSMD−1.9, effect size −0.62) in the combined MDD/BPD population.

### Study 6: Post-Hoc Analysis for Impact on Sexual Functioning

A post hoc analysis of Study 6 investigated the impact of lumateperone tosylate 60 mg on sexual function, assessed using the Changes in Sexual Functioning Questionnaire, 14-item version (CSFQ-14), in patients with MDD, including patients with baseline sexual dysfunction. As described above, the primary and key secondary efficacy endpoints in Study 6 were change from baseline to Day 43 in MADRS Total score and CGI-S score, respectively, in the modified intent-to-treat (mITT) population, analyzed using a mixed-effects model for repeated measures (MMRM). Sexual function was evaluated based on changes in CSFQ-14 from baseline to day 43 in the intent-to-treat (ITT) population, using an MMRM. A change from placebo of 2-3 points on CSFQ-14 total score is considered clinically meaningful.

The CSFQ is a questionnaire used to systematically assess sexual function in patients. The original CSFQ existed in a 36 or 35-question version, but more commonly a short-form 14-question version is now used (the CSFQ-14). Each question is answered by the patient on a scale from 1 to 5, resulting in a total CSFQ-14 score ranging from 14 to 70. Higher score numbers indicate more normal sexual function, while lower scores indicate increasing sexual dysfunction. The questions are commonly grouped into five subscales: (1) Pleasure (Question 1); (2) Desire/Frequency (Questions 2 and 3); (3) Desire/Interest (Questions 4, 5, and 6); (4) Arousal/Excitement (Questions 7, 8, and 9); and (5) Orgasm/Completion (Questions 11, 12, and 13). Questions 10 and 14 are not included in any of these five subscales.

A total CSFQ-14 score of 47 or less indicates sexual dysfunction in males, while a total score of 41 or less indicates sexual dysfunction in females. Because the number of questions included in each subscale varies, indications of dysfunction in each subscale are based on different cut-off values, as follows:

| | Dysfunction | Dysfunction |
|---|---|---|
| | threshold in males | threshold in females |
| CSFQ-14 Total | ≤47 | ≤41 |
| Pleasure subscale | ≤4 | ≤4 |
| Desire/Frequency subscale | ≤8 | ≤6 |
| Desire/Interest subscale | ≤11 | ≤9 |
| Arousal/Excitement subscale | ≤13 | ≤12 |
| Orgasm/Completion subscale | ≤13 | ≤11 |

In the Study 6 ITT patient population, the baseline mean CSFQ-14 measures and rates of sexual dysfunction were as follows:

| | Lumateperone + | Placebo + |
|---|---|---|
| Baseline | ADT (n = 242) | ADT (n = 238) |
| CSFQ-14 Total Score, mean | 34.7 | 34.0 |
| Female patients | 31.7 | 31.1 |
| Male patients | 41.8 | 40.6 |
| Patients with sexual | 82% | 83% |
| dysfunction | | |
| Female patients | 59% | 60% |
| Male patients | 23% | 24% |
| CSFQ-14 Total Score, mean, | 31.4 | 31.0 |
| in patients with sexual | | |
| dysfunction | | |
| Female patients | 28.7 | 28.5 |
| Male patients | 38.3 | 37.2 |
| CSFQ-14 Subscales, means | | |
| Pleasure | 2.0 | 1.8 |
| Desire/Frequency | 4.3 | 4.1 |
| Desire/Interest | 5.9 | 5.8 |
| Arousal/Excitement | 7.0 | 6.8 |
| Orgasm/Completion | 7.3 | 7.0 |

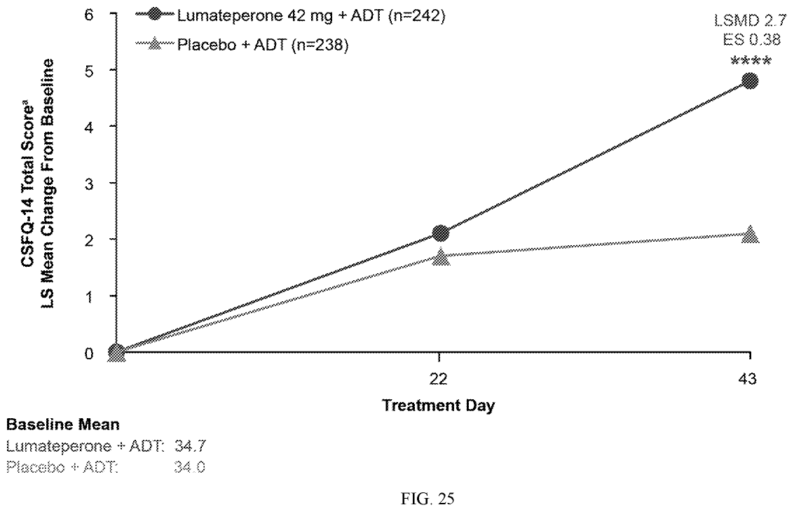

It was unexpectedly found that lumateperone+ADT significantly improved the CSFQ-14 total score compared with placebo+ADT at Day 43 in the ITT population (LSMD 2.7; effect size 0.38). The results are shown in FIG. 25. The results were much stronger in woman than in men. In women, lumateperone+ADT significantly improved the CSFQ-14 total score compared with placebo+ADT at Day 43 (LSMD 3.5; effect size 0.47; P<0.0001), but in men the improvement was substantial but not statistically significant (LSMD 1.9; effect size 0.33; P=0.079).

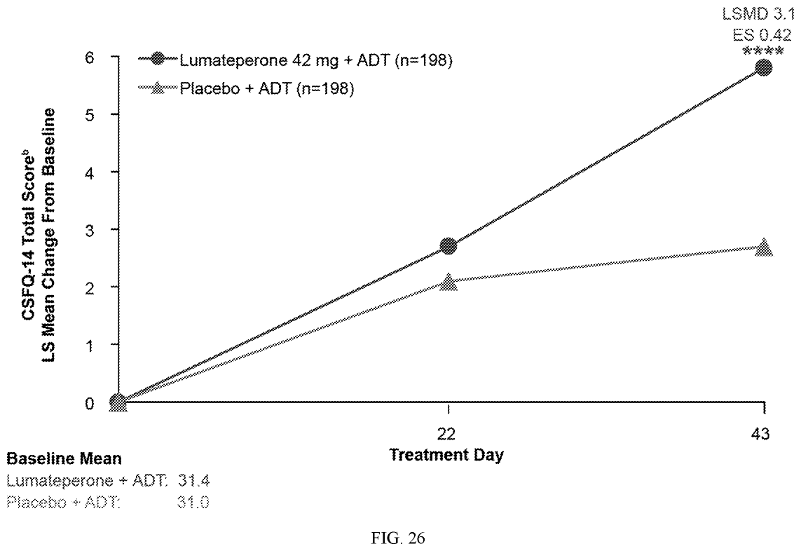

Of the 480 patients in the Study 6 ITT population, 396 had sexual dysfunction at baseline (83%), based on CSFQ-14 total score. The effect of lumateperone treatment on CSFQ-14 total score was even more pronounced in the sexual dysfunction subgroup than in the ITT population (LSMD 3.1; effect size 0.42). The results are shown in FIG. 26. Similar to the ITT population results, these results were also stronger in woman than in men. In women with sexual dysfunction at baseline, lumateperone+ADT significantly improved the CSFQ-14 total score compared with placebo+ADT at Day 43 (LSMD 3.8; effect size 0.50; P<0.0001), but in men the improvement was substantial but not statistically significant (LSMD 2.2; effect size 0.36; P=0.0975).

Interestingly, in patients without sexual dysfunction at baseline, there was no change in CSFQ-14 at Day 43. In patients without sexual dysfunction (17% of ITT), the mean baseline CSFQ-14 total score was 50.1 in the lumateperone+ADT group, and 49.1 in the placebo+ADT group. At Day 43, the mean CSFQ-14 total score LSMD was −0.4, with an effect size of −0.10 (P=0.73). Thus, lumateperone unexpectedly improves sexual functioning in patients with baseline sexual dysfunction, but does not affect sexual dysfunction in patients without baseline dysfunction.

Results were also compared in the ITT population between those patients younger than 45 years old (18-45 years old), and those 45 years old or older (45-65 years old). The results were substantially the same in both groups (<45 years: LSMD 3.0, effect size 0.41; ≥45 years: LSMD 3.0, effect size 0.43).

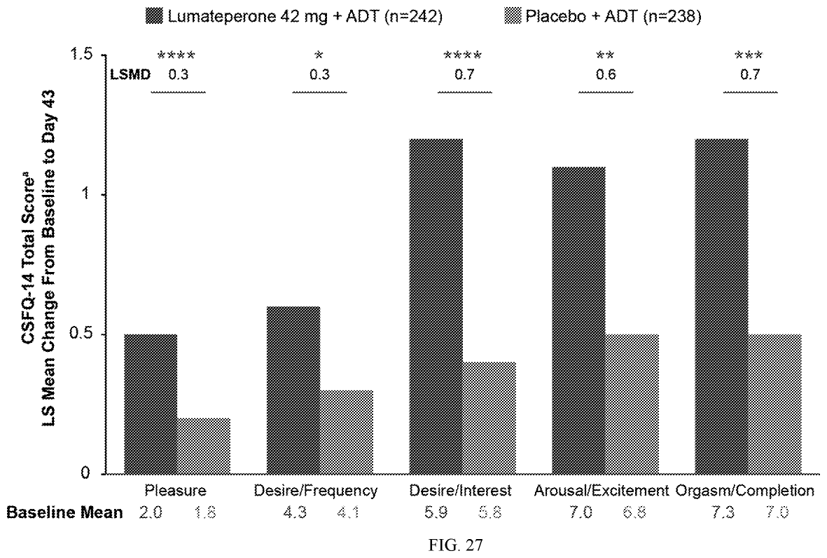

Patients were also analyzed for the effects of lumateperone on the individual CSFQ-14 subscale scores. Within the ITT population, lumateperone+ADT significantly improved each of the CSFQ-14 subscale scores compared with placebo+ADT at Day 43. The results are shown in FIG. 27. The results were unexpectedly more pronounced for the pleasure, desire/interest, and orgasm/completion subscales.

The preceding examples are merely exemplary and do not limit the scope of the present invention.

1. A method for the treatment of major depressive disorder (MDD), comprising the administration of pharmaceutical capsules comprising 42 mg of lumateperone (60 mg of lumateperone tosylate) once daily to a patient in need thereof, provided that if the patient has moderate to severe hepatic impairment or treatment is concomitant with a moderate CYP 3A4 inhibitor, the patient is administered pharmaceutical capsules comprising 21 mg of lumateperone (30 mg of lumateperone tosylate) once daily, and if treatment is concomitant with a strong CYP 3A4 inhibitor, then the patient is administered pharmaceutical capsules comprising 10.5 mg of lumateperone (15 mg of lumateperone tosylate) once daily; wherein the lumateperone is administered in the form of lumateperone tosylate; wherein the method is adjunctive to therapy with antidepressants.

2. The method of claim 1, wherein the patient has no hepatic impairment or mild hepatic impairment, and/or wherein the patient is not concomitantly administered a CYP 3A4 inhibitor, and the patient is administered 42 mg of lumateperone (60 mg of lumateperone tosylate) daily.

3. The method of claim 1, wherein the patient has or is diagnosed with moderate to severe hepatic impairment, and the patient is administered 21 mg of lumateperone (30 mg of lumateperone tosylate) daily.

4. The method of claim 1, wherein the patient is concomitantly administered a moderate CYP 3A4 inhibitor, and the patient is administered 21 mg of lumateperone (30 mg of lumateperone tosylate) daily.

5. The method of claim 1, wherein the patient is concomitantly administered a strong CYP 3A4 inhibitor, and the patient is administered 10.5 mg of lumateperone (15 mg of lumateperone tosylate) daily.

6. The method of claim 1, wherein the capsule further comprises croscarmellose sodium, gelatin, magnesium stearate, mannitol, talc, and colorants.

7. The method of claim 1, wherein the patient had an inadequate response to previous antidepressant therapy, e.g., antidepressant therapy of at least 6 weeks duration.

8. The method of claim 7, wherein the previous antidepressant therapy is selected from an SSRI, SNRI, or bupropion-patient.

9. The method of claim 7, wherein the SSRI is selected from citalopram, escitalopram, fluoxetine, fluvoxamine, paroxetine, and sertraline.

10. The method of claim 7, wherein the SNRI is selected from desvenlafaxine, duloxetine, levomilnacipran, milnacipran, tofenacin, and venlafaxine.

11. The method of claim 7, wherein the inadequate response is characterized by less than a 50% improvement in symptoms (e.g., as measured by MADRS total score and/or CGI-S score and/or by patient questionnaire evaluation).

12. The method of claim 1, wherein the MDD is characterized by major depressive episodes with (a) anxious distress, (b) mixed features, (c) melancholic features, (d) atypical features, (e) psychotic features, (f) catatonia, (g) peripartum onset, or (h) seasonal pattern.

13. The method of claim 1, wherein the administration of the lumateperone is adjunct to treatment with an antidepressant agent selected from an SSRI, SNRI, vortioxetine, bupropion, or mirtazapine.

14. The method of claim 1, wherein the patient is switching from treatment with another therapeutic agent for the treatment of MDD, to treatment with lumateperone (lumateperone tosylate), wherein the switch does not include dose titration.

15. The method of claim 1, wherein the method provides a significant reduction in the MADRS total score within 42 days of the beginning of treatment.

16. The method of claim 1, wherein the method provides a significant reduction in the MADRS total score within 21 days of the beginning of treatment.

17. The method of claim 1, wherein the method provides a significant reduction in the MADRS total score within 14 days of the beginning of treatment.

18. The method of claim 1, wherein the method provides a significant reduction in the MADRS total score within 7 days of the beginning of treatment.

19. The method of claim 1, wherein the patient suffers from an anxiety disorder or wherein the patient suffers from MDD with anxious distress, wherein the generalized anxiety disorder or anxious distress is characterized by GAD-7 total score of greater than or equal to 10, and/or wherein the patient experiences 2 or more anxious symptoms (e.g., feeling tense, feeling restless, difficulty concentrating because of worry, fearful something awful may happen, or feeling out of control) during the majority of days during a major depressive episode.

20. The method of claim 1, wherein the method provides a reduction in the MADRS total score within 43 days of the beginning of treatment of at least 10 points.

21. A method for achieving remission of major depressive disorder (MDD), comprising the administration of pharmaceutical capsules comprising 42 mg of lumateperone (60 mg of lumateperone tosylate) once daily to a patient in need thereof, provided that if the patient has moderate to severe hepatic impairment or treatment is concomitant with a moderate CYP 3A4 inhibitor, the patient is administered pharmaceutical capsules comprising 21 mg of lumateperone (30 mg of lumateperone tosylate) once daily, and if treatment is concomitant with a strong CYP 3A4 inhibitor, then the patient is administered pharmaceutical capsules comprising 10.5 mg of lumateperone (15 mg of lumateperone tosylate) once daily; wherein the method is adjunctive to therapy with antidepressants.

22. A method for achieving complete remission of major depressive disorder (MDD), comprising the administration of pharmaceutical capsules comprising 42 mg of lumateperone (60 mg of lumateperone tosylate) once daily to a patient in need thereof, provided that if the patient has moderate to severe hepatic impairment or treatment is concomitant with a moderate CYP 3A4 inhibitor, the patient is administered pharmaceutical capsules comprising 21 mg of lumateperone (30 mg of lumateperone tosylate) once daily, and if treatment is concomitant with a strong CYP 3A4 inhibitor, then the patient is administered pharmaceutical capsules comprising 10.5 mg of lumateperone (15 mg of lumateperone tosylate) once daily; wherein the method is adjunctive to therapy with antidepressants.

23. A method for achieving sustained remission of major depressive disorder (MDD), comprising the administration of pharmaceutical capsules comprising 42 mg of lumateperone (60 mg of lumateperone tosylate) once daily to a patient in need thereof, provided that if the patient has moderate to severe hepatic impairment or treatment is concomitant with a moderate CYP 3A4 inhibitor, the patient is administered pharmaceutical capsules comprising 21 mg of lumateperone (30 mg of lumateperone tosylate) once daily, and if treatment is concomitant with a strong CYP 3A4 inhibitor, then the patient is administered pharmaceutical capsules comprising 10.5 mg of lumateperone (15 mg of lumateperone tosylate) once daily; wherein the method is adjunctive to therapy with antidepressants.

24. The method of claim 1, wherein the patient is diagnosed with MDD according to the DSM-5-TR criteria, or according to DSM-IV-TR criteria, or according to the DSM-5 criteria.

25. The method of claim 1, wherein the MDD is characterized by major depressive episodes characterized by an MADRS total score of greater than or equal to 24 and/or a CGI-S score of greater than or equal to 4.

26. The method of claim 25, wherein the MDD is further characterized a Quick Inventory of Depressive Symptomology-Self Report-16 Item (QIDS-SR-16) score of greater than or equal to 14.

27. The method of claim 1, wherein the patient has a baseline MADRS Total score of less than or equal to 32, at the start of treatment.

28. The method of claim 1, wherein the patient is an adult.
